(19) United States
(12) Patent Application Publication
(10) Pub. No.: US 2024/0279627 A1
(43) Pub. Date: Aug. 22, 2024

(54) COMPOSITIONS COMPRISING A LIPOLYTIC ENZYME VARIANT AND METHODS OF USE THEREOF

(71) Applicant: DANISCO US INC, Palo Alto, CA (US)

(72) Inventors: Despina BOUGIOUKOU, Wilmington, DE (US); Robert DICOSIMO, Chadds Ford, PA (US); Angel Elizabeth Grace D'OLIVIERA, Wilmington, DE (US); Jonathan David PETERSON, Egg Harbor Township, NJ (US); Jahnavi Chandra PRASAD, Wilmington, DE (US); Brian F. SCHMIDT, Palo Alto, CA (US); Joshua Joyner SIMS, Philadelphia, PA (US)

(21) Appl. No.: 17/270,220

(22) PCT Filed: Aug. 19, 2019
(86) PCT No.: PCT/US2019/047035

§ 371 Date: Feb. 22, 2021

Related U.S. Application Data
(60) Provisional application No. 62/724,877, filed on Aug. 30, 2018.

(51) Int. Cl. C12N 9/20; C11D 3/386

(52) U.S. Cl. CPC C12N 9/20; C11D 3/38627; C12Y 301/01003

(57) ABSTRACT

The present disclosure provides lipolytic enzyme variants having one or more modifications as compared to a parent lipolytic enzyme. Specifically, the present disclosure provides one or more lipolytic enzyme variants that has at least one improved performance when compared to one or more reference lipolytic enzymes. In addition, the present disclosure provides compositions comprising a lipolytic enzyme variant of the disclosure. The present disclosure also provides methods of cleaning using compositions comprising a lipolytic enzyme variant of the disclosure.

This application claims the benefit of U.S. Provisional Application No. 62/724,877 filed Aug. 30, 2018, incorporated herein in its entirety by reference.

### FIELD

The disclosure relates to lipolytic enzyme variants, polynucleotides encoding the variants, methods of producing the variants, and methods of using the variants.

### REFERENCE TO SEQUENCE LISTING SUBMITTED ELECTRONICALLY

The official copy of the sequence listing is submitted electronically via EFS-Web as an ASCII formatted sequence listing with a file named 20190816_NB41299PCT_SeqLst.txt created on Aug. 16, 2019 and having a size 15 kilobytes and is filed concurrently with the specification. The sequence listing contained in this ASCII formatted document is part of the specification and is herein incorporated by reference in its entirety.

### BACKGROUND

Lipolytic enzymes have been employed in detergent cleaning compositions for the removal of oily stains. One mechanism by which lipolytic enzymes function is by hydrolyzing triglycerides to generate fatty acids. However, these enzymes are often inhibited by surfactants and other components present in cleaning composition, interfering with their ability to remove oily stains. Accordingly, the need exists for lipolytic enzymes that can function in the harsh environment of cleaning compositions.

### SUMMARY

The present disclosure provides one or more lipolytic enzyme variants, and compositions and methods related to the production and use thereof, including one or more lipolytic enzyme variants that has at least one improved performance when compared to one or more reference lipolytic enzymes. Specifically, the present disclosure provides lipolytic enzyme variants having one or more modifications, such as a substitution, as compared to a parent lipolytic enzyme. The improved performance of the one or more lipolytic enzyme variants or active fragments thereof relative to the parent lipolytic enzyme, can be an improved wash performance, a decreased malodor, an increased detergent stability, an increased thermostability, an increased calcium ion binding stability, an increased protease stability, or any one combination thereof. The present disclosure provides lipolytic enzyme variants that are particularly well suited to and useful in a variety of cleaning applications. The disclosure also provides methods of cleaning using lipolytic enzyme variants of the present disclosure.

In one embodiment, the disclosure provides a lipolytic enzyme variant or an active fragment thereof comprising an amino acid modification to a parent lipolytic enzyme, wherein the amino acid modification is at a position of the lipolytic enzyme variant selected from the group consisting 2, 4, 11, 13, 14, 17, 18, 19, 20, 22, 27, 28, 29, 30, 31, 33, 43, 44, 46, 47, 49, 50, 52, 53, 57, 60, 62, 64, 65, 67, 68, 70, 83, 85, 91, 93, 95, 99, 105, 117, 118, 119, 120, 121, 122, 125, 127, 128, 130, 131, 132, 134, 135, 137, 138, 140, 141, 146, 147, 150, 154, 155, 159, 160, 164, 166, 170, 171, 175, 178, 179, 180, 185, 189, 196, 199, 203, 204, 205, 206, 208, 209, 212, 217, 218, 221, 223, 224, 227, 229, 247, 251, 252, 255, 258, 261, 262, 264, 267, 268, 272 and 280, wherein the amino acid positions of the variant are numbered by correspondence with the amino acid sequence of SEQ ID NO: 2, wherein the lipolytic enzyme variant has at least 60% sequence identity to SEQ ID NO:1 or 2. In one aspect, the lipolytic enzyme variant is derived from a parent lipolytic enzyme having 60%, 65%, 70%, 75%, 80%, 85%, 86%, 87%, 88%, 89%, 90%, 91%, 92%, 93%, 94%, 95%, 96%, 97%, 98%, 99%, or 100% amino acid sequence identity to the amino acid sequence of SEQ ID NO: 1 or 2. In one aspect, the variant comprises an amino acid sequence having 60%, 65%, 70%, 75%, 80%, 85%, 86%, 87%, 88%, 89%, 90%, 91%, 92%, 93%, 94%, 95%, %, 97%, 98% or 99% amino acid sequence identity to the amino acid sequence of SEQ ID NO: 1 or 2.

In one embodiment, the disclosure provides a lipolytic enzyme variant or an active fragment thereof comprising an amino acid modification to a parent lipolytic enzyme, wherein the amino acid modification is a substitution selected from the group consisting of S002V, T4K, H011A, H011K, L013C, L013F, L013W, S014C, S014G, S014L, S014W, D017A, D017G, D017S, D017T, D018A, D018F, D018G, D018I, D018K, D018L, D018M, D018N, D018P, D018R, D018T, D018V, D018W, 1019C, V020G, Y022F, Y022I, Y022V, Y022W, G027K, 1028V, A29R, D030A, D030K, D030R, A031G, E033I, E033K, S043K, S043R, L044Q, A046G, A046S, A046T, F047C, F047Y, S049D, S049T, N050P, V052F, V052K, V052M, R053I, R053M, R053Q, R053T, R053V, L057C, F060K, Q062A, 164V, L065H, E067I, E067Q, E067R, E067S, E067T, T068S, A070V, A070Y, L083M, C085A, C085G, K091D, K091E, K091N, K091Q, A093E, S095P, S095N, V099I, V105A, V105C, V105P, R117A, R117C, R117D, R117E, R117G, R117I, R117K, R117N, R117Q, R117S, R117T, R117V, I118D, I118E, I118G, I118H, I118N, I118S, M119C, M119V, M119Y, R120A, R120C, R120E, R120F, R120G, R120H, R120I, R120K, R120L, R120M, R120Q, R120T, R120V, R120W, K121A, K121E, K121N, K121Q, K121S, K121T, D122P, P125D, P125E, P125F, Y127C, Y127D, Y127E, Y127G, Y127H, Y127N, I128A, I128C, I128G, I128K, I128M, I128N, I128Q, I128T, I128Y, A129S, D130C, A131D, A131E, A131N, A131P, A131Q, A131S, A131T, V132T, K134C, K134D, K134E, K134F, K134G, K134I, K134M, K134N, K134Q, K134S, K134T, K134V, K134W, K134Y, A135E, G137R, T138I, T138V, I140E, I140F, I140T, I140V, S141M, N146M, N146H, R147P, P150D, P150E, P150T, I154M, I154N, I154Q, I154T, I154V, A155E, A155M, A159D, A159S, L160A, L160C, L160S, L160T, L160V, N164A, N164E, N164Q, N164S, N164R, N164D, M166A, M166D, M166E, K170A, K170D, K170H, K170Q, K170T, K171C, K171D, K171E, K171Q, G175A, A178D, A178K, I179E, I179K, R180E, R180P, K185C, K185D, K185E, K185L, K185S, N189D, F196I, Y199F, L203C, L203K, L203N, L203P, L203R, L203S, L203T, I204Y, A205C, A205F, A205I, A205L, A205M, A205P, A205V, A205W, A205Y, G206C, G206D, G206E, G206Q, K208E, K208G, K208M, K208Q, G209A, G209C, G209D, G209E, G209Q, L212K, L212R, A217F, A217I, A217L, A217V, A217Y, A218C, V221F, V221T, S223G, A224G, A224H, A224K, A224Q, A224R, A224S, A224W, A224Y, S227D, S227I, S227N, S227T, S227V, R229A, R229C, R229D, R229E, R229G, R229H, R229I, R229K, R229L, R229M, R229N, R229P, R229Q, R229S, R229T, R229V, K247R, A251P, E252Q, L255C, V258I, V261L, A262C, A262F, A262L, A262M, L264K, R267P, G268D, G268K, I272G, I272K, I272P, I272R, I272V and N280K, wherein the amino acid positions of the variant are numbered by correspondence with the amino acid sequence of SEQ ID NO: 2 wherein the lipolytic enzyme variant has at least 60%, 65%, 70%, 75%, 80%, 85%, 86%, 87%, 88%, 89%, 90%, 91%, 92%, 93%, 94%, 95%, 96%, 97%, 98%, 99%, or 100% amino acid sequence identity to SEQ ID NO: 1 or 2.

In one embodiment, the disclosure provides a lipolytic enzyme variant or active fragment thereof comprising an amino acid modification to a parent lipolytic enzyme, wherein said lipolytic enzyme variant or active fragment thereof has an improved performance relative to the parent lipolytic enzyme, wherein the improved performance is selected from the group consisting of an improved wash performance, a decreased malodor, an increased detergent stability, an increased thermostability, an increased calcium ion binding stability, an increased protease stability, or any one combination thereof. The lipolytic enzyme variant or active fragment thereof having an improved wash performance can be a lipolytic enzyme variant that has a wash performance index (PI(wash)) relative to the parent lipolytic enzyme that is greater than 1.0. The lipolytic enzyme variant or active fragment thereof having an increased thermostability can be a lipolytic enzyme variant or active fragment thereof that has a thermostability performance index (PI(thermostability)) relative to the parent lipolytic enzyme that is greater than 1.0. The lipolytic enzyme variant or active fragment thereof having an increased detergent stability (such as increased stability in liquid detergent) can be a lipolytic enzyme variant or active fragment thereof that has a detergent stability performance index (PI(detergent stability)) relative to the parent lipolytic enzyme that is greater than 1.0. The lipolytic enzyme variant or active fragment thereof having an increased calcium ion stability can be a lipolytic enzyme variant or active fragment thereof that has a calcium ion stability index (PI(calcium ion stability)) relative to the parent lipolytic enzyme that is greater than 1.0. The lipolytic enzyme variant or active fragment thereof can be a variant or active fragment that has a protease stability that is greater that the protease stability of the parent lipolytic enzyme.

In one embodiment, the disclosure provides a lipolytic enzyme variant or active fragment thereof, wherein the variant or active fragment has a decreased malodor performance index (PI(malodor) relative to the lipolytic enzyme of SEQ ID NO: 7 that is smaller than 1.0.

In one embodiment, the lipolytic enzyme variants of the disclosure have a protease stability that is greater than the protease stability of the parent lipolytic enzyme.

Some further embodiments are directed to a composition comprising one or more lipolytic enzyme variants described herein. Further embodiments are directed to a method of cleaning comprising contacting a surface or an item in need of cleaning with one or more lipolytic enzyme variants described herein or one or more compositions described herein.

Still other embodiments are directed to a method for producing a variant described herein, comprising stably transforming a host cell with an expression vector comprising a polynucleotide encoding one or more lipolytic enzyme variants described herein. Still further embodiments are directed to a polynucleotide comprising a nucleic acid sequence encoding one or more lipolytic enzyme variants described herein.

### BRIEF DESCRIPTION OF THE SEQUENCE LISTING

The disclosure can be more fully understood from the following detailed description and the accompanying Sequence Listing, which forms a part of this application. The sequence descriptions and sequence listing attached hereto comply with the rules governing nucleotide and amino acid sequence disclosures in patent applications as set forth in 37 C.F.R. §§ 1.821 1.825. The sequence descriptions contain the three letter codes for amino acids as defined in 37 C.F.R. §§ 1.821 1.825, which are incorporated herein by reference.

### DETAILED DESCRIPTION

The present disclosure provides one or more lipolytic enzyme variants, and compositions and methods related to the production and use thereof, including one or more lipolytic enzyme variants that has at least one improved performance (improved property) when compared to one or more reference lipolytic enzymes. Specifically, the present disclosure provides lipolytic enzyme variants having one or more modifications, such as a substitution, as compared to a parent lipolytic enzyme. The improved performance of one or more lipolytic enzyme variants or active fragments thereof relative to the parent lipolytic enzyme, can be an improved wash performance, a decreased malodor, an increased detergent stability, an increased thermostability, an increased calcium ion binding stability, an increased protease stability, or any one combination thereof. The present disclosure provides lipolytic enzyme variants, including, but not limited to, variant lipase lipolytic enzymes, that are particularly well suited to and useful in a variety of cleaning applications. The disclosure includes compositions comprising at least one of the lipolytic enzyme variants (e.g., variant lipases) set forth herein. Some such compositions comprise detergent compositions. The lipolytic enzyme variants of the present disclosure can be combined with other enzymes useful in detergent compositions.

The disclosure also provides enzyme compositions having a comparable or improved performance, as compared to known lipolytic enzymes, such as, known lipase lipolytic enzymes (WO2014/059360, WO2015/010009, WO2018/015295). The disclosure also provides methods of cleaning using lipolytic enzyme variants of the present disclosure.

The disclosure includes enzyme variants of lipolytic enzymes having one, two, three, four, five, six, seven, eight, nine or ten or more modifications from a parent lipolytic enzyme. The enzyme variants can be useful in a composition, such as but not limiting to a detergent composition, by having an improved performance relative to a parent lipolytic enzyme, wherein the improved performance is selected from the group consisting of an improved wash performance, a decreased malodor, an increased detergent stability, an increased thermostability, an increased calcium ion binding stability, an increased protease stability, or any one combination thereof.

The disclosure includes enzyme variants of lipolytic enzymes having one or more modifications from a parent lipolytic enzyme. These amino acid modifications can result in an improved performance of the variant and their amino acid positions can be considered useful positions for combinatorial modifications to a parent lipolytic enzyme. Lipolytic enzyme amino acid positions found to be useful positions can be further characterized by having multiple modifications that are suitable for use in a composition, such as but not limiting to a detergent composition.

Unless otherwise indicated herein, one or more lipolytic enzyme variants described herein can be made and used via conventional techniques commonly used in molecular biology, microbiology, protein purification, protein engineering, protein and DNA sequencing, recombinant DNA fields, and industrial enzyme use and development.

Terms and abbreviations not defined should be accorded their ordinary meaning as used in the art. Unless defined otherwise herein, all technical and scientific terms used herein have the same meaning as commonly understood by one of ordinary skill in the art. Although many methods and materials similar or equivalent to those described herein find use in the practice of the present disclosure, some methods and materials are described herein. All patents, patent applications, articles and publications mentioned herein, both supra and infra, are hereby expressly incorporated herein by reference.

As used herein, the singular terms “a,” “an,” and “the” include the plural reference unless the context clearly indicates otherwise. Unless otherwise indicated, nucleic acids are written left to right in 5′ to 3′ orientation; amino acid sequences are written left to right in amino to carboxy orientation, respectively. It is to be understood that this disclosure is not limited to the particular methodology, protocols, and reagents described, as these may vary, depending upon the context in which they are used by those of skill in the art.

It is intended that every maximum numerical limitation given throughout this specification include every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

The terms “derived from” and “obtained from” refer not only to a lipolytic enzyme produced or producible by a strain of the organism in question, but also a lipolytic enzyme encoded by a DNA sequence isolated from such strain and produced in a host organism containing such DNA sequence. Additionally, the term refers to a lipolytic enzyme which is encoded by a DNA sequence of synthetic and/or cDNA origin and which has the identifying characteristics of the lipolytic enzyme in question. To exemplify, “lipolytic enzymes derived from Proteus” refers to those enzymes having lipolytic activity which are naturally produced by Proteus, as well as to lipolytic enzymes like those produced by Proteus sources but which through the use of genetic engineering techniques are produced by non-Proteus organisms transformed with a nucleic acid encoding the lipolytic enzymes.

The term “vector” refers to a nucleic acid construct used to introduce or transfer nucleic acid(s) into a target cell or tissue. A vector is typically used to introduce foreign DNA into a cell or tissue. Vectors include plasmids, cloning vectors, bacteriophages, viruses (e.g., viral vector), cosmids, expression vectors, shuttle vectors, and the like. A vector typically includes an origin of replication, a multicloning site, and a selectable marker. The process of inserting a vector into a target cell is typically referred to as transformation.

As used herein in the context of introducing a nucleic acid sequence into a cell, the term “introduced” refers to any method suitable for transferring the nucleic acid sequence into the cell. Such methods for introduction include but are not limited to protoplast fusion, transfection, transformation, electroporation, conjugation, and transduction. Transformation refers to the genetic alteration of a cell which results from the uptake, optional genomic incorporation, and expression of genetic material (e.g., DNA).

The term “expression” refers to the transcription and stable accumulation of sense (mRNA) or anti-sense RNA, derived from a nucleic acid molecule of the disclosure. Expression may also refer to translation of mRNA into a polypeptide. Thus, the term “expression” includes any step involved in the “production of the polypeptide” including, but not limited to, transcription, post-transcriptional modifications, translation, post-translational modifications, secretion and the like.

The phrases “expression cassette” or “expression vector” refers to a nucleic acid construct or vector generated recombinantly or synthetically for the expression of a nucleic acid of interest (e.g., a foreign nucleic acid or transgene) in a target cell. The nucleic acid of interest typically expresses a protein of interest. An expression vector or expression cassette typically comprises a promoter nucleotide sequence that drives or promotes expression of the foreign nucleic acid. The expression vector or cassette also typically includes other specified nucleic acid elements that permit transcription of a particular nucleic acid in a target cell. A recombinant expression cassette can be incorporated into a plasmid, chromosome, mitochondrial DNA, plastid DNA, virus, or nucleic acid fragment. Some expression vectors have the ability to incorporate and express heterologous DNA fragments in a host cell or genome of the host cell. Many prokaryotic and eukaryotic expression vectors are commercially available. Selection of appropriate expression vectors for expression of a protein from a nucleic acid sequence incorporated into the expression vector is within the knowledge of those of skill in the art.

As used herein, a nucleic acid is “operably linked” with another nucleic acid sequence when it is placed into a functional relationship with another nucleic acid sequence. For example, a promoter or enhancer is operably linked to a nucleotide coding sequence if the promoter affects the transcription of the coding sequence. A ribosome binding site may be operably linked to a coding sequence if it is positioned so as to facilitate translation of the coding sequence. Typically, “operably linked” DNA sequences are contiguous. However, enhancers do not have to be contiguous. Linking is accomplished by ligation at convenient restriction sites. If such sites do not exist, synthetic oligonucleotide adaptors or linkers may be used in accordance with conventional practice.

The term “gene” refers to a polynucleotide (e.g., a DNA segment), that encodes a polypeptide and includes regions preceding and following the coding regions. In some instances, a gene includes intervening sequences (introns) between individual coding segments (exons).

The terms “host strain” and “host cell” refer to a suitable host for an expression vector comprising a DNA sequence of interest.

The term “recombinant”, when used with reference to a cell typically indicates that the cell has been modified by the introduction of a foreign nucleic acid sequence or that the cell is derived from a cell so modified. For example, a recombinant cell may comprise a gene not found in identical form within the native (non-recombinant) form of the cell, or a recombinant cell may comprise a native gene (found in the native form of the cell) that has been modified and re-introduced into the cell. A recombinant cell may comprise a nucleic acid endogenous to the cell that has been modified without removing the nucleic acid from the cell; such modifications include those obtained by gene replacement, site-specific mutation, and related techniques known to those of ordinary skill in the art. Recombinant DNA technology includes techniques for the production of recombinant DNA in vitro and transfer of the recombinant DNA into cells where it may be expressed or propagated, thereby producing a recombinant polypeptide. “Recombination” and “recombining” of polynucleotides or nucleic acids refer generally to the assembly or combining of two or more nucleic acid or polynucleotide strands or fragments to generate a new polynucleotide or nucleic acid.

A nucleic acid or polynucleotide is said to “encode” a polypeptide if, in its native state or when manipulated by methods known to those of skill in the art, it can be transcribed and/or translated to produce the polypeptide or a fragment thereof. The anti-sense strand of such a nucleic acid is also said to encode the sequence.

A “protein” or “polypeptide” comprises a polymeric sequence of amino acid residues. The terms “protein” and “polypeptide” are used interchangeably herein. The single and 3-letter code for amino acids as defined in conformity with the IUPAC-IUB Joint Commission on Biochemical Nomenclature (JCBN) is used through out this disclosure. It is also understood that a polypeptide can be coded for by more than one nucleotide sequence due to the degeneracy of the genetic code. Mutations can be named by the one letter code for the parent amino acid, followed by a number and then the one letter code for the variant amino acid. For example, mutating glycine (G) at position 87 to serine (S) can be represented as “G087S” or “G87S”. Multiple mutations can be indicated by inserting a “_” between the mutations. For example, mutations at positions 87 and 90 can be represented as either “G087S_A090Y” or “G87S_A90Y” or “G87S+A90Y” or “G087S+A090Y”.

The terms “signal sequence” and “signal peptide” refer to a sequence of amino acid residues that may participate in the secretion or direct transport of the mature or precursor form of a protein. The signal sequence is typically located N-terminal to the precursor or mature protein sequence. The signal sequence may be endogenous or exogenous. A signal sequence is normally absent from the mature protein. A signal sequence is typically cleaved from the protein by a signal peptidase after the protein is transported.

The term “mature” form of a protein, polypeptide, or peptide refers to the functional form of the protein, polypeptide, or peptide without the signal peptide sequence and propeptide sequence.

The term “precursor” form of a protein or peptide refers to a mature form of the protein having a prosequence operably linked to the amino or carbonyl terminus of the protein. The precursor may also have a “signal” sequence operably linked to the amino terminus of the prosequence. The precursor may also have additional polypeptides that are involved in post-translational activity (e.g., polypeptides cleaved therefrom to leave the mature form of a protein or peptide).

The term “identical” in the context of two polynucleotide or polypeptide sequences refers to the nucleic acids or amino acids in the two sequences that are the same when aligned for maximum correspondence, as measured using sequence comparison or analysis algorithms described below and known in the art.

The phrases “% identity” or “percent identity” or “percent sequence identity” or “PID” in the context of two or more nucleic acid or polypeptide sequences refers to two or more sequences that are the same or have a specified percentage of nucleic acid residues or amino acid residues, respectively, that are the same, when compared and aligned for maximum similarity, as determined using a sequence comparison algorithm or by visual inspection. Percent identity may be determined using standard techniques known in the art. The percent amino acid identity shared by sequences of interest can be determined by aligning the sequences to directly compare the sequence information, e.g., by using a program such as BLAST, MUSCLE, or CLUSTAL. The BLAST algorithm is described, for example, in Altschul et al., J Mol Biol, 215:403-410 (1990) and Karlin et al., Proc Natl Acad Sci USA, 90:5873-5787(1993). A percent (%) amino acid sequence identity value is determined by the number of matching identical residues divided by the total number of residues of the “reference” sequence including any gaps created by the program for optimal/maximum alignment. BLAST algorithms refer to the “reference” sequence as the “query” sequence. Software for performing BLAST analyses is publicly available through the National Center for Biotechnology Information. This algorithm involves first identifying high scoring sequence pairs (HSPs) by identifying short words of length W in the query sequence that either match or satisfy some positive-valued threshold score T when aligned with a word of the same length in a database sequence. These initial neighborhood word hits act as starting points to find longer HSPs containing them. The word hits are expanded in both directions along each of the two sequences being compared for as far as the cumulative alignment score can be increased. Extension of the word hits is stopped when: the cumulative alignment score falls off by the quantity X from a maximum achieved value; the cumulative score goes to zero or below; or the end of either sequence is reached. The BLAST algorithm parameters W, T, and X determine the sensitivity and speed of the alignment. The BLAST program uses as defaults a wordlength (W) of 11, the BLOSUM62 scoring matrix (See, Henikoff and Henikoff, Proc. Natl. Acad. Sci. USA 89:10915 (1992)) alignments (B) of 50, expectation (E) of 10, M′5, N′-4, and a comparison of both strands.

The BLAST algorithm then performs a statistical analysis of the similarity between two sequences (See e.g., Karlin and Altschul, supra). One measure of similarity provided by the BLAST algorithm is the smallest sum probability (P(N)), which provides an indication of the probability by which a match between two nucleotide or amino acid sequences would occur by chance. For example, a nucleic acid is considered similar to a lipolytic enzyme nucleic acid of this disclosure if the smallest sum probability in a comparison of the test nucleic acid to a lipolytic enzyme nucleic acid is less than about 0.1, more preferably less than about 0.01, and most preferably less than about 0.001. Where the test nucleic acid encodes a lipolytic enzyme polypeptide, it is considered similar to a specified lipolytic enzyme nucleic acid if the comparison results in a smallest sum probability of less than about 0.5, and more preferably less than about 0.2.

The CLUSTAL W algorithm is another example of a sequence alignment algorithm (See, Thompson et al., Nucleic Acids Res, 22:4673-4680, 1994). Default parameters for the CLUSTAL W algorithm include: Gap opening penalty=10.0; Gap extension penalty=0.05; Protein weight matrix=BLOSUM series; DNA weight matrix=IUB; Delay divergent sequences %=40; Gap separation distance=8; DNA transitions weight=0.50; List hydrophilic residues=GPSNDQEKR; Use negative matrix=OFF; Toggle Residue specific penalties=ON; Toggle hydrophilic penalties=ON; and Toggle end gap separation penalty=OFF. In CLUSTAL algorithms, deletions occurring at either terminus are included. For example, a variant with a five amino acid deletion at either terminus (or within the polypeptide) of a polypeptide of 500 amino acids would have a percent sequence identity of 99% (495/500 identical residues×100) relative to the “reference” polypeptide. Such a variant would be encompassed by a variant having “at least 99% sequence identity” to the polypeptide.

Percent sequence identity” or “% identity” or “% sequence identity or “% amino acid sequence identity” of a subject amino acid sequence to a reference (i.e., query) amino acid sequence means that the subject amino acid sequence is identical (i.e., on an amino acid-by-amino acid basis) by a specified percentage to the query amino acid sequence over a comparison length when the sequences are optimally aligned. Thus, 80% amino acid sequence identity or 80% identity with respect to two amino acid sequences means that 80% of the amino acid residues in two optimally aligned amino acid sequences are identical.

“Percent sequence identity” or “% identity” or “% sequence identity or “% nucleotide sequence identity” of a subject nucleic acid sequence to a reference (i.e. query) nucleic acid sequence means that the subject nucleic acid sequence is identical (i.e., on a nucleotide-by-nucleotide basis for a polynucleotide sequence) by a specified percentage to the query sequence over a comparison length when the sequences are optimally aligned. Thus, 80% nucleotide sequence identity or 80% identity with respect to two nucleic acid sequences means that 80% of the nucleotide residues in two optimally aligned nucleic acid sequences are identical.

“Optimal alignment” or “optimally aligned” refers to the alignment of two (or more) sequences giving the highest percent identity score. For example, optimal alignment of two protein sequences can be achieved by manually aligning the sequences such that the maximum number of identical amino acid residues in each sequence are aligned together or by using software programs or procedures described herein or known in the art. Optimal alignment of two nucleic acid sequences can be achieved by manually aligning the sequences such that the maximum number of identical nucleotide residues in each sequence are aligned together or by using software programs or procedures described herein or known in the art.

In some aspects, two polypeptide sequences are deemed “optimally aligned” when they are aligned using defined parameters, such as a defined amino acid substitution matrix, gap existence penalty (also termed gap open penalty), and gap extension penalty, so as to achieve the highest similarity score possible for that pair of sequences. The BLOSUM62 scoring matrix (See, Henikoff and Henikoff, supra) is often used as a default scoring substitution matrix in polypeptide sequence alignment algorithms (e.g., BLASTP). The gap existence penalty is imposed for the introduction of a single amino acid gap in one of the aligned sequences, and the gap extension penalty is imposed for each residue position in the gap. Exemplary alignment parameters employed are: BLOSUM62 scoring matrix, gap existence penalty=11, and gap extension penalty=1. The alignment score is defined by the amino acid positions of each sequence at which the alignment begins and ends (e.g., the alignment window), and optionally by the insertion of a gap or multiple gaps into one or both sequences, so as to achieve the highest possible similarity score.

Optimal alignment between two or more sequences can be determined manually by visual inspection or by using a computer, such as, but not limited to for example, the BLASTP program for amino acid sequences and the BLASTN program for nucleic acid sequences (See e.g., Altschul et al., Nucleic Acids Res. 25(17):3389-3402 (1997); See also, the National Center for Biotechnology Information (NCBI) website).

A polypeptide of interest may be said to be “substantially identical” to a parent polypeptide if the polypeptide of interest comprises an amino acid sequence having at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 99.5% sequence identity to the amino acid sequence of the parent polypeptide. The percent identity between two such polypeptides can be determined manually by inspection of the two optimally aligned polypeptide sequences or by using software programs or algorithms (e.g., BLAST, ALIGN, CLUSTAL) using standard parameters. One indication that two polypeptides are substantially identical is that the first polypeptide is immunologically cross-reactive with the second polypeptide. Typically, polypeptides that differ by conservative amino acid substitutions are immunologically cross-reactive. Thus, a polypeptide is substantially identical to a second polypeptide, for example, where the two peptides differ only by a conservative amino acid substitution or one or more conservative amino acid substitutions.

A nucleic acid of interest may be said to be “substantially identical” to a parent nucleic acid if the nucleic acid of interest comprises a nucleotide sequence having at least about 60%, at least about 65% at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 99.5% sequence identity to the nucleotide sequence of the parent nucleic acid. The percent identity between two such nucleic acids can be determined manually by inspection of the two optimally aligned nucleic acid sequences or by using software programs or algorithms (e.g., BLAST, ALIGN, CLUSTAL) using standard parameters. One indication that two nucleic acid sequences are substantially identical is that the two nucleic acid molecules hybridize to each other under stringent conditions (e.g., within a range of medium to high stringency).

As used herein, “homologous proteins” or “homologous lipolytic enzymes” or “homologous lipases” refers to proteins that have distinct similarity in primary, secondary, and/or tertiary structure. Protein homology can refer to the similarity in linear amino acid sequence when proteins are aligned. Homology can be determined by amino acid sequence alignment, e.g., using a program such as BLAST, MUSCLE, or CLUSTAL. Homologous search of protein sequences can be done using BLASTP and PSI-BLAST from NCBI BLAST with threshold (E-value cut-off) at 0.001. (Altschul et al., “Gapped BLAST and PSI BLAST a new generation of protein database search programs”, Nucleic Acids Res, Set 1; 25(17):3389-402(1997)). The BLAST program uses several search parameters, most of which are set to the default values. The NCBI BLAST algorithm finds the most relevant sequences in terms of biological similarity but is not recommended for query sequences of less than 20 residues (Altschul et al., Nucleic Acids Res, 25:3389-3402, 1997 and Schaffer et al., Nucleic Acids Res, 29:2994-3005, 2001). Exemplary default BLAST parameters for a nucleic acid sequence searches include: Neighboring words threshold=11; E-value cutoff 10; Scoring Matrix=NUC.3.1 (match=1, mismatch=−3); Gap Opening=5; and Gap Extension=2. Exemplary default BLAST parameters for amino acid sequence searches include: Word size=3; E-value cutoff=10; Scoring Matrix=BLOSUM62; Gap Opening=11; and Gap extension=1. Using this information, protein sequences can be grouped and/or a phylogenetic tree built therefrom. Amino acid sequences can be entered in a program such as the Vector NTI Advance suite and a Guide Tree can be created using the Neighbor Joining (NJ) method (Saitou and Nei, Mol Biol Evol, 4:406-425, 1987). The tree construction can be calculated using Kimura's correction for sequence distance and ignoring positions with gaps. A program such as AlignX can display the calculated distance values in parenthesis following the molecule name displayed on the phylogenetic tree.

When 3-dimensional structures of proteins have been determined, structurally homologous amino acid positions between two or more molecules can be determined. When there are significant structural similarities among these molecules it might be expected that introducing substitutions that confer improvement in one molecule at structurally homologous sites in another molecule could confer similar improvements in performance and/or stability to these molecules. Structurally homologous amino acid positions can be identified by performing a structural alignment, which attempts to determine homology between two or more protein structures based on their shape and three-dimensional conformation. Structural alignment can produce a superposition of the atomic coordinate sets and a minimal root mean square deviation between the structures. Examples of methods for creating structural alignments are the distance alignment matrix method (DALI) (Holm L, Sander C (1996) “Mapping the protein universe”. Science. 273 (5275): 595-603), combinatorial extension (CE) (Shindyalov, I. N.; Bourne P. E. (1998) “Protein structure alignment by incremental combinatorial extension (CE) of the optimal path”. Protein Engineering. 11 (9): 739-747), and Sequential Structure Alignment Program (SSAP) (Taylor W R, Flores T P, Orengo C A (1994) “Multiple protein structure alignment”. Protein Sci. 3 (10): 1858-70). By combining multiple sequence alignments with structural alignments, structurally homologous amino acid positions can be identified in molecules for which the 3-dimensional structure has not been determined. Examples of methods for creating multiple sequence alignment-based structural alignments are 3DCoffee (Poirot O et al (2004) “3DCoffee®igs: a web server for combining sequences and structures into a multiple sequence alignment” Nucleic Acids Res. 2004 Jul. 1; 32:W37-40), PROMALS3D (Pei J et al. (2008) “PROMALS3D: a tool for multiple protein sequence and structure alignments.” Nucleic Acids Res. 36(7):2295-300), and 3DM (Kuipers, R K et al (2010) “3DM: Systematic analysis of heterogeneous superfamily data to discover protein functionalities” Proteins 78(9):2101-13). Understanding the homology between molecules can reveal the evolutionary history of the molecules as well as information about their function; if a newly sequenced protein is homologous to an already characterized protein, there is a strong indication of the new protein's biochemical function. The most fundamental relationship between two entities is homology; two molecules are said to be homologous if they have been derived from a common ancestor. Homologous molecules, or homologs, can be divided into two classes, paralogs and orthologs. Paralogs are homologs that are present within one species. Paralogs often differ in their detailed biochemical functions. Orthologs are homologs that are present within different species and have very similar or identical functions. A protein superfamily is the largest grouping (clade) of proteins for which common ancestry can be inferred. Usually this common ancestry is based on sequence alignment and mechanistic similarity. Superfamilies typically contain several protein families which show sequence similarity within the family.

A nucleic acid or polynucleotide is “isolated” when it is partially or completely separated from other components, including but not limited to for example, other proteins, nucleic acids, cells, etc. Similarly, a polypeptide, protein or peptide is “isolated” when it is partially or completely separated from other components, including but not limited to for example, other proteins, nucleic acids, cells, etc. On a molar basis, an isolated species is more abundant than are other species in a composition. For example, an isolated species may comprise at least about 50%, about 70%, about 80%, about 85%, about 90%, about 91%, about 92%, about 93%, about 94%, about 95%, about 96%, about 97%, about 98%, about 99%, or about 100% (on a molar basis) of all macromolecular species present. Preferably, the species of interest is purified to essential homogeneity (i.e., contaminant species cannot be detected in the composition by conventional detection methods). Purity and homogeneity can be determined using a number of techniques well known in the art, such as agarose or polyacrylamide gel electrophoresis of a protein or nucleic acid sample, followed by visualization upon staining. If desired, a high-resolution technique, such as high performance liquid chromatography (HPLC) or a similar means can be utilized for purification of the material.

The term “purified” as applied to nucleic acids or polypeptides generally denotes a nucleic acid or polypeptide that is essentially free from other components as determined by analytical techniques well known in the art (e.g., a purified polypeptide or polynucleotide forms a discrete band in an electrophoretic gel, chromatographic eluate, and/or a media subjected to density gradient centrifugation). For example, a nucleic acid or polypeptide that gives rise to essentially one band in an electrophoretic gel is “purified.” A purified nucleic acid or polypeptide is at least about 50% pure, usually at least about 75%, about 80%, about 85%, about 90%, about 91%, about 92%, about 93%, about 94%, about 95%, about 96%, about 97%, about 98%, about 99%, about 99.5%, about 99.6%, about 99.7%, about 99.8% or more pure (e.g., percent by weight on a molar basis). In a related sense, a composition is enriched for a molecule when there is a substantial increase in the concentration of the molecule after application of a purification or enrichment technique. The term “enriched” refers to a compound, polypeptide, cell, nucleic acid, amino acid, or other specified material or component that is present in a composition at a relative or absolute concentration that is higher than a starting composition. A substantially pure polypeptide or polynucleotide of the disclosure (e.g., substantially pure lipolytic enzyme variant or polynucleotide encoding a lipolytic enzyme variant of the disclosure, respectively) will typically comprise at least about 55%, about 60%, about 70%, about 80%, about 85%, about 90%, about 91%, about 92%, about 93%, about 94%, about 95%, about 96%, about 97%, about 98, about 99%, about 99.5% or more by weight (on a molar basis) of all macromolecular species in a particular composition.

The term “effective amount” of one or more lipolytic enzyme variants described herein or reference lipolytic enzyme(s) refers to the amount of lipolytic enzyme that achieves a desired level of enzymatic activity in a specific composition, such as but not limited to a cleaning composition. Such effective amounts are readily ascertained by one of ordinary skill in the art and are based on many factors, such as the particular lipolytic enzyme used, the cleaning application, the specific composition of the cleaning composition, and whether a liquid or dry (e.g., granular, tablet, bar, pods) or single-unit dose composition is required.

The term “adjunct material” refers to any liquid, solid, or gaseous material included in cleaning composition other than one or more lipolytic enzyme variants described herein, or recombinant polypeptide or active fragment thereof. In some embodiments, the cleaning compositions of the present disclosure include one or more cleaning adjunct materials. Each cleaning adjunct material is typically selected depending on the particular type and form of cleaning composition (e.g., liquid, granule, powder, bar, paste, spray, tablet, gel, foam, or other composition). Preferably, each cleaning adjunct material is compatible with the lipolytic enzyme used in the composition.

### Lipolytic Enzyme Variants and Parent Enzymes

The present disclosure provides one or more lipolytic enzyme variants having one or more modifications as compared to a parent lipolytic enzyme.

As used herein, a “lipolytic enzyme”, “lipolytic polypeptide” and “lipolytic protein” refers to an enzyme, polypeptide, or protein exhibiting a lipid-degrading capability such as a capability of degrading a triglyceride or a phospholipid. The lipolytic enzyme can be, for example, a lipase, a phospholipase, an esterase or a cutinase. Lipolytic enzymes can be lipolytic enzymes having an α/β hydrolase fold. These enzymes typically have a catalytic triad of serine, aspartic acid and histidine residues. The α/β hydrolases include lipases and cutinases. Cutinases show little, if any, interfacial activation, where lipases often undergo a conformational change in the presence of a lipid-water interface (Longhi and Cambillau (1999) Biochimica et Biophysica Acta 1441:185-96). An active fragment of a lipolytic enzyme is a portion of a lipolytic enzyme that retains a lipid degrading capability (lipolytic activity). An active fragment retains the catalytic triad. In one aspect, an active fragment of a parent or variant lipase contains at least 50%, at least 55%, at least 60%, at least 65%, at least 70%, at least 75%, at least 80%, at least 85%, at least 90%, or at least 95% but less than 100% of the number of the amino acids present in the parent ot variant lipase. As used herein, lipolytic activity can be determined according to any procedure known in the art (see, e.g., Gupta et al., Biotechnol. Appl. Biochem., 37:63-71, 2003; U.S. Pat. No. 5,990,069; and International Patent Publication No. WO 96/18729A1).

In some embodiments, lipolytic enzymes of the present disclosure are a/0 hydrolases. In some embodiments, lipolytic enzymes of the present disclosure are lipases. In some embodiments, lipolytic enzymes of the present disclosure are cutinases.

The term “parent”, “parent lipolytic enzyme” and “parent lipase” refers to a lipolytic enzyme to which a modification (substitution, deletion or insertion) is made to produce the enzyme variants of the present disclosure. The parent may be a synthetic polypeptide, a naturally occurring (wild-type) polypeptide, or a variant or fragment thereof.

In some embodiments the parent lipolytic enzyme is a lipolytic enzyme derived from the Genus Proteus. In one aspect the parent lipolytic enzyme comprises or consists of the amino acid sequence of SEQ ID NO: 1. In one aspect the parent lipolytic enzyme comprises or consists of the amino acid sequence of SEQ ID NO: 2.

SEQ ID NO:1 sets forth the amino acid sequence of the parent lipase derived from Proteus sp. H24: MSTTYPIVLVHGLSGFDDIVGYPYFYGIADALEKDGHKVFTASLSAFNSNEVRGEQLWE FVQKILKETKVKKVNLIGHSQGPLACRYVAAKHAKSIASVTSVNGVNHGSEIADLVRRI MRKDSVPEYIADAVMKAIGTIISTFSGNRGNPQDAIAALEALTTENVMEFNKKYPQGLP AIRGGEGKEVVNGVHYYSFGSYIQGLIAGEKGNLLDPTHAAMRVLSAFFSERENDGLV GRTSMRLGKLIKDDYAEDHLDMVNQVAGLVGRGEDIIAIYTNHANFLASKKL SEQ ID NO:2 sets forth the amino acid sequence of the parent lipase derived from Proteus vulgaris (WP_099659650.1):

MSTTYPIVLVHGLSGFDDIVGYPYFYGIADALEKDGHKVFTASLSA
FNSNEVRGEQLWEFVQKILKETKAKKVNLIGHSQGPLACRYVAAK
HAKSIASVTSVNGVNHGSEIADLVRRIMRKDSVPEYIADAVMKAI
GTIISTFSGNRGNPQDAIAALEALTTENVMEFNKKYPQGLPAIRG
GEGKEVVNGVHYYSFGSYIQGLIAGEKGNLLDPTHAAMRVLSAFF
SERENDGLVGRTSMRLGKLIKDDYAEDHLDMVNQVAGLVGRGEDI
IAIYTNHANFLASKKL

Parent lipases SEQ ID NO: 1 and SEQ ID NO:2 differ from each other in one amino acid. SEQ ID NO: 1 has a valine at position 70 while SEQ ID NO: 2 has an alanine at position 70.

The term “lypolytic enzyme variant”, “lipase variant”, “variant” and “lipolytic variant” refers to a polypeptide having lipolytic activity comprising at least one modification, i.e., a substitution, insertion, and/or deletion, at one or more (e.g., several) positions, when compared to a parent lipolytic enzyme. A substitution means replacement of the amino acid occupying a position with a different amino acid; a deletion means removal of the amino acid occupying a position; and an insertion means adding an amino acid adjacent to and immediately following the amino acid occupying a position.

In some embodiments, the lipolytic enzyme variant is derived from a parent lipolytic enzyme from the Genus Proteus.

In some embodiments, the lipolytic enzyme variant is derived from a parent lipolytic enzyme having 60%, 65%, 70%, 75%, 80%, 85%, 86%, 87%, 88%, 89%, 90%, 91%, 92%, 93%, 94%, 95%, 96%, 97%, 98%, 99%, or 100% amino acid sequence identity to the amino acid sequence of SEQ ID NO: 1 or 2.

In some embodiments, the lipolytic enzyme variant comprises an amino acid sequence having 60%, 65%, 70%, 75%, 80%, 85%, 86%, 87%, 88%, 89%, 90%, 91%, 92%, 93%, 94%, 95%, 96%, 97%, 98% or 99% amino acid sequence identity to the amino acid sequence of SEQ ID NO: 1 or 2.

The position of an amino acid residue in a given amino acid sequence is numbered herein using the numbering of the position of the corresponding amino acid residue of a reference lipolytic enzyme. Unless otherwise indicated, the position of an amino acid residue in a given amino acid sequence is numbered by correspondence with the amino acid sequence of SEQ ID NO: 2. For purposes of the present disclosure, SEQ ID NO: 2 is used to determine the corresponding amino acid residue in another lipolytic enzyme. The amino acid sequence of SEQ ID NO:2, thus serves as a reference sequence. A given amino acid sequence, such as a lipolytic enzyme variant amino acid sequence described herein, can be aligned with the reference lipolytic amino acid sequence of SEQ ID NO: 2 using an alignment algorithm as described herein, and each amino acid residue in the given amino acid sequence that aligns (preferably optimally aligns) with an amino acid residue in the reference lipolytic amino acid sequence of SEQ ID NO: 2 is conveniently numbered by reference to the numerical position of that corresponding amino acid residue. Sequence alignment algorithms, such as, for example, those described herein will identify the location where insertions or deletions occur in a subject sequence when compared to a query sequence. In one aspect the reference lipolytic enzyme is the parent lipolytic enzyme of SEQ ID NO: 2.

The nomenclature of the amino acid substitutions of the one or more lipolytic enzyme variants described herein uses one or more of the following: position; position:amino acid or amino acid substitution(s); or starting amino acid(s):position:amino acid substitution(s). For example, the substitution of threonine at position 4 with lysine is designated as “T4K”. Reference to a “position” (i.e. 4, 29, 64, etc) encompasses any starting amino acid that may be present at such position, and any substitution that may be present at such position. Reference to a position can be recited several forms, for example, position 003 can also be referred to as position 3. Reference to a “position: amino acid substitution(s)” (i.e. 1S/T/G, 3G, 17T, etc) encompasses any starting amino acid that may be present at such position and the one or more amino acid(s) with which such starting amino acid may be substituted. Reference to a starting or substituted amino acid may be further expressed as several starting, or substituted amino acids separated by a foreslash (“/”). For example, D275S/K indicates position 275 is substituted with serine (S) or lysine (K) and P/S197K indicates that starting amino acid proline (P) or serine (S) at position 197 is substituted with lysine (K). Reference to an X as the amino acid in a position, refers to any amino acid at the recited position.

For an amino acid deletion, the following nomenclature is used: starting amino acid: position, *. For example, the deletion of threonine at position 4 is designated as “T4*”.

For an amino acid insertion, the following nomenclature is used: starting amino acid:position:starting amino acid:inserted amino acid. For example, the insertion of glycine after threonine at position 4 is designated as “T4TG”.

Multiple modifications are separated by an underscore “_” or addition marks “+”. For example, the multiple substitution of threonine at position 4 with lysine together with the substitution of leucine at position 230 with arginine is designated as T4K_L203R or T4K+L203R.

The term “wild-type” lipolytic enzyme means a lipolytic enzyme expressed by a naturally occurring microorganism, such as a bacterium, yeast, or filamentous fungus found in nature.

In one embodiment, the disclosure provides a lipolytic enzyme variant or an active fragment thereof comprising an amino acid modification to a parent lipolytic enzyme, wherein the amino acid modification is at a position of the lipolytic enzyme variant selected from the group consisting of 2, 4, 11, 13, 14, 17, 18, 19, 20, 22, 27, 28, 29, 30, 31, 33, 43, 44, 46, 47, 49, 50, 52, 53, 57, 60, 62, 64, 65, 67, 68, 70, 83, 85, 91, 93, 95, 99, 105, 117, 118, 119, 120, 121, 122, 125, 127, 128, 130, 131, 132, 134, 135, 137, 138, 140, 141, 146, 147, 150, 154, 155, 159, 160, 164, 166, 170, 171, 175, 178, 179, 180, 185, 189, 196, 199, 203, 204, 205, 206, 208, 209, 212, 217, 218, 221, 223, 224, 227, 229, 247, 251, 252, 255, 258, 261, 262, 264, 267, 268, 272 and 280, wherein the amino acid positions of the variant are numbered by correspondence with the amino acid sequence of SEQ ID NO: 2, wherein the lipolytic enzyme variant has at least 60%, 65%, 70%, 75%, 80%, 85%, 86%, 87%, 88%, 89%, 90%, 91%, 92%, 93%, 94%, 95%, 96%, 97%, 98%, 99%, or 100% amino acid sequence identity to SEQ ID NO: 1 or 2.

In one embodiment, the disclosure provides a lipolytic enzyme variant or an active fragment thereof comprising an amino acid modification to a parent lipolytic enzyme, wherein the amino acid modification is at a position of the lipolytic enzyme variant selected from the group consisting of S002V, T4K, H011A, H011K, L013C, L013F, L013W, S014C, S014G, S014L, S014W, D017A, D017G, D017S, D017T, D018A, D018F, D018G, D018I, D018K, D018L, D018M, D018N, D018P, D018R, D018T, D018V, D018W, I019C, V020G, Y022F, Y022I, Y022V, Y022W, G027K, I028V, A29R, D030A, D030K, D030R, A031G, E033I, E033K, S043K, S043R, L044Q, A046G, A046S, A046T, F047C, F047Y, S049D, S049T, N050P, V052F, V052K, V052M, R053I, R053M, R053Q, R053T, R053V, L057C, F060K, Q062A, I64V, L065H, E067I, E067Q, E067R, E067S, E067T, T068S, A070V, A070Y, L083M, C085A, C085G, K091D, K091E, K091N, K091Q, A093E, S095P, S095N, V099I, V105A, V105C, V105P, R117A, R117C, R117D, R117E, R117G, R117I, R117K, R117N, R117Q, R117S, R117T, R117V, I118D, I118E, I118G, I181H, 118N, I118S, M119C, M119V, M119Y, R120A, R120C, R120E, R120F, R120G, R120H, R120I, R120K, R120L, R120M, R120Q, R120T, R120V, R120W, K121A, K121E, K121N, K121Q, K121S, K121T, D122P, P125D, P125E, P125F, Y127C, Y127D, Y127E, Y127G, Y127H, Y127N, I128A, I128C, I128G, I128K, I128M, I128N, I128Q, I128T, I128Y, A129S, D130C, A131D, A131E, A131N, A131P, A131Q, A131S, A131T, V132T, K134C, K134D, K134E, K134F, K134G, K134I, K134M, K134N, K134Q, K134S, K134T, K134V, K134W, K134Y, A135E, G137R, T138I, T138V, I140E, I140F, I140T, I140V, S141M, N146M, N146H, R147P, P150D, P150E, P150T, I154M, I154N, I154Q, I154T, I154V, A155E, A155M, A159D, A159S, L160A, L160C, L160S, L160T, L160V, N164A, N164E, N164Q, N164S, N164R, N164D, M166A, M166D, M166E, K170A, K170D, K170H, K170Q, K170T, K171C, K171D, K171E, K171Q, G175A, A178D, A178K, I179E, I179K, R180E, R180P, K185C, K185D, K185E, K185L, K185S, N189D, F196I, Y199F, L203C, L203K, L203N, L203P, L203R, L203S, L203T, I204Y, A205C, A205F, A205I, A205L, A205M, A205P, A205V, A205W, A205Y, G206C, G206D, G206E, G206Q, K208E, K208G, K208M, K208Q, G209A, G209C, G209D, G209E, G209Q, L212K, L212R, A217F, A217I, A217L, A217V, A217Y, A218C, V221F, V221T, S223G, A224G, A224H, A224K, A224Q, A224R, A224S, A224W, A224Y, S227D, S227I, S227N, S227T, S227V, R229A, R229C, R229D, R229E, R229G, R229H, R229I, R229K, R229L, R229M, R229N, R229P, R229Q, R229S, R229T, R229V, K247R, A251P, E252Q, L255C, V258I, V261L, A262C, A262F, A262L, A262M, L264K, R267P, G268D, G268K, I272G, I272K, I272P, I272R, I272V and N280K, wherein the amino acid positions of the variant are numbered by correspondence with the amino acid sequence of SEQ ID NO:2 wherein the lipolytic enzyme variant has at least 60%, 65%, 70%, 75%, 80%, 85%, 86%, 87%, 88%, 89%, 90%, 91%, 92%, 93%, 94%, 95%, 96%, 97%, 98%, 99%, or 100% amino acid sequence identity to SEQ ID NO: 1 or 2.

Lipolytic Enzyme Variants with Improved Performance

The present disclosure provides one or more lipolytic enzyme variants having one or more modifications as compared to a parent lipolytic enzyme, including lipolytic enzyme variants having an improved performance compared to a parent or reference lipolytic enzyme.

As used herein, the term “improved property” (plural “improved properties”) and “improved performance” of a lipolytic enzyme variant refers to a characteristic associated with a lipolytic enzyme variant that is improved relative to the corresponding parent lipolytic enzyme. Such improved properties include, but are not limited to an improved cleaning performance (such as improved wash performance), an increased detergent stability, an increased expression, a decreased (reduced) malodor, an increased thermostability, an increased calcium ion binding stability, an increased protease stability, or any one combination thereof.

In some aspects, the lipolytic enzyme variants of the disclosure have an improved wash performance. The term “wash performance” and “cleaning performance” refers to a cleaning performance achieved by lipolytic enzyme or reference lipase under conditions prevailing during the lipolytic, hydrolyzing, cleaning, or other process of the disclosure. In some embodiments, cleaning performance of a lipolytic enzyme or reference lipase may be determined by using various assays for cleaning one or more lipase-sensitive stains on an item or surface (e.g., beef fat, butterfat, lard, margarine, vegetable oil, bacon grease, sebum). Cleaning performance of one or more lipolytic enzyme variants described herein or reference lipase can be determined by subjecting the stain on the item or surface to standard wash condition(s) and assessing the degree to which the stain is removed by using various chromatographic, spectrophotometric, or other quantitative methodologies.

In one aspect, the lipolytic variant has an improved wash performance, wherein the the lipolytic variant has a wash performance index (PI(wash)) relative to a parent lipolytic enzyme that is greater than 1.0. In some aspects, the wash performance is compared under relevant washing conditions. In some test systems, other relevant factors, such as detergent composition, suds concentration, water hardness, washing mechanics, time, pH, and/or temperature, can be controlled in such a way that condition(s) typical for household application in a certain market segment (e.g., hand or manual dishwashing, automatic dishwashing, dishware cleaning, tableware cleaning, fabric cleaning, etc.) are imitated.

In some embodiments, one or more compositions described herein comprises one or more lipolytic enzyme variants described herein as useful or effective for cleaning a surface in need of oily-stain removal.

In some embodiments, one or more lipolytic enzyme variants described herein cleans at low temperatures, also referred to as cold temperatures. In other embodiments, one or more compositions described herein cleans at low temperatures. In one aspect, the lipolytic variant has an improved wash performance, wherein the improved wash performance is an improved wash performance at a low temperature. Low temperatures include cleaning temperatures of 10° C., 11° C., 12° C., 13° C., 14° C., 15° C., 16° C., 17° C., 18° C., 19° C., 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C. and up to 40° C. Low temperatures include cleaning or washing temperatures from about 10° C. to about 40° C., or from about 20° C. to about 30° C., or from about 15° C. to about 25° C., as well as all other combinations within the range of about 15° C. to about 35° C., and all ranges within 10° C. to 40° C.

In one aspect, the lipolytic variant has an improved wash performance, wherein the improved wash performance is an improved wash performance during cold water washing. Cold water washing of the present disclosure utilizes cold water detergent comprising one or more lipolytic enzyme variants described herein, suitable for washing at temperatures from about 10° C. to about 40° C., or from about 20° C. to about 30° C., or from about 15° C. to about 25° C., as well as all other combinations within the range of about 15° C. to about 35° C., and all ranges within 10° C. to 40° C.

In one aspect, the lipolytic variant has an improved wash performance, wherein the improved wash performance is at a cleaning temperature of 40° C.

In some aspects, the lipolytic enzyme variants of the disclosure have an increased stability. The term “enhanced stability” or “improved stability” in the context of an oxidation, chelator, denaturant, surfactant, thermal and/or pH stable lipolytic enzyme refers to a higher retained lipolytic activity over time as compared to a reference lipolytic enzyme, for example, a parent lipase. The stability of lipolytic enzymes of the present disclosure can be compared to the stability of a standard, for example, but not limited to, the parent lipolytic enzyme of SEQ ID NO: 1 or 2 In one aspect the lipolytic enzyme variants of the disclosure have an equal stability compared to the parent lipolytic enzyme of SEQ ID NO: 1 or 2, combined with an improved property such as but not limiting to improved cleaning performance (such as improved wash performance), an increased expression, a decreased (reduced) malodor, an increased detergent stability, an increased thermostability, an increased calcium ion binding stability, an increased protease stability, or any one combination thereof.

In some aspects, the lipolytic enzyme variants of the disclosure have an increased detergent stability. As used herein, the term “enhanced detergent stability” or “increased detergent stability” refers to lipolytic enzyme variants of the present disclosure that when present in a detergent composition, retain a higher amount of enzymatic activity over time as compared to a reference lipolytic enzyme, for example, a parent lipase. In some aspects the lipolytic enzyme variants of the disclosure have an increased detergent stability relative to a parent lipase, such as but not limited to the parent lipase shown in SEQ ID NO:1 or 2.

The relative increase in detergent stability is expressed as a performance index (PI), where PI (detergent stability)=% residual activity variant/% residual activity of parent lipase SEQ ID NO:2. In some aspects, the lipolytic enzyme variants of the disclosure have a detergent stability index (PI(detergent stability)) relative to the parent lipolytic enzyme that is greater than 1.0.

In some aspects, the detergent stability of a lipolytic enzyme or variant thereof is assayed in a liquid detergent (such as but not limiting to a so-called heavy-duty liquid (HDL) detergent) and the PI (detergent stability) determined as described herein.

In one embodiment, the disclosure provides a lipolytic enzyme variant or an active fragment thereof comprising an amino acid modification to a parent lipolytic enzyme, wherein the amino acid modification is at a position of the lipolytic enzyme variant selected from the group consisting of 2, 4, 11, 13, 14, 17, 18, 19, 20, 22, 27, 28, 29, 30, 31, 33, 43, 44, 46, 47, 49, 50, 52, 53, 57, 60, 62, 65, 67, 68, 70, 83, 85, 91, 93, 95, 99, 105, 117, 118, 119, 120, 121, 125, 127, 128, 130, 131, 132, 134, 135, 137, 138, 140, 141, 146, 147, 150, 154, 155, 159, 160, 164, 166, 170, 171, 175, 178, 180, 185, 189, 196, 199, 203, 204, 205, 206, 208, 209, 212, 217, 218, 221, 223, 224, 227, 229, 247, 251, 252, 255, 258, 261, 262, 264, 268, 272 and 280, wherein the amino acid positions of the variant are numbered by correspondence with the amino acid sequence of SEQ ID NO: 2, wherein the lipolytic enzyme variant has at least 60% sequence identity to SEQ ID NO: 1 or 2, and wherein the variant or active fragment has a detergent stability index (PI(detergent stability)) relative to the parent lipolytic enzyme that is greater than 1.0.

In one embodiment, the disclosure provides a lipolytic enzyme variant or an active fragment thereof comprising an amino acid modification to a parent lipolytic enzyme, wherein the amino acid modification is at a position of the lipolytic enzyme variant selected from the group consisting of S002V, T4K, H011A, H011K, L013C, L013F, L013W, S014C, S014G, S014L, S014W, D017A, D017G, D017S, D017T, D018A, D018F, D018G, D018I, D018K, D018L, D018M, D018N, D018P, D018R, D018T, D018V, D018W, I019C, V020G, Y022F, Y022I, Y022V, Y022W, G027K, I028V, A29R, D030A, D030K, D030R, A031G, E033I, E033K, S043K, S043R, L044Q, A046G, A046S, A046T, F047C, F047Y, S049D, S049T, N050P, V052F, V052K, V052M, R053I, R053M, R053Q, R053T, R053V, L057C, F060K, Q062A, L065H, E067I, E067Q, E067R, E067S, E067T, T068S, A070V, A070Y, L083M, C085A, C085G, K091D, K091E, K091N, K091Q, A093E, S095P, S095N, V099I, V105A, V105C, V105P, R117A, R117C, R117D, R117E, R117G, R117I, R117K, R117N, R117Q, R117S, R117T, R117V, I118D, I118E, I118G, I118H, I118N, I18S, M119C, M119V, M119Y, R120A, R120C, R120E, R120F, R120G, R120H, R120I, R120K, R120L, R120M, R120Q, R120T, R120V, R120W, K121A, K121E, K121N, K121Q, K121S, K121T, P125D, P125E, P125F, Y127C, Y127D, Y127E, Y127G, Y127H, Y127N, I128A, I128C, I128G, I128K, I128M, I128N, I128Q, I128T, I128Y, A129S, D130C, A131D, A131E, A131N, A131P, A131Q, A131S, A131T, V132T, K134C, K134D, K134E, K134F, K134G, K134I, K134M, K134N, K134Q, K134S, K134T, K134V, K134W, K134Y, A135E, G137R, T138I, T138V, I140E, I140F, I140T, I140V, S141M, N146M, N146H, R147P, P150D, P150E, P150T, I154M, I154N, I154Q, I154T, I154V, A155E, A155M, A159D, A159S, L160A, L160C, L160S, L160T, L160V, N164A, N164E, N164Q, N164S, N164R, N164D, M166A, M166D, M166E, K170A, K170D, K170H, K170Q, K170T, K171C, K171D, K171E, K171Q, G175A, A178D, A178K, R180E, R180P, K185C, K185D, K185E, K185L, K185S, N189D, F196I, Y199F, L203C, L203K, L203N, L203P, L203R, L203S, L203T, I204Y, A205C, A205F, A205I, A205L, A205M, A205P, A205V, A205W, A205Y, G206C, G206D, G206E, G206Q, K208E, K208G, K208M, K208Q, G209A, G209C, G209D, G209E, G209Q, L212K, L212R, A217F, A217I, A217L, A217V, A217Y, A218C, V221F, V221T, S223G, A224G, A224H, A224K, A224Q, A224R, A224S, A224W, A224Y, S227D, S227I, S227N, S227T, S227V, R229A, R229C, R229D, R229E, R229G, R229H, R229I, R229K, R229L, R229M, R229N, R229P, R229Q, R229S, R229T, R229V, K247R, A251P, E252Q, L255C, V258I, V261L, A262C, A262F, A262L, A262M, L264K, G268D, G268K, I272G, I272K, I272P, I272R, I272V and N280K, wherein the lipolytic enzyme variant has at least 60% sequence identity to SEQ ID NO: 1 or 2, wherein the amino acid positions of the variant are numbered by correspondence with the amino acid sequence of SEQ ID NO: 2, and wherein the variant or active fragment has a detergent stability index (PI(detergent stability)) relative to the parent lipolytic enzyme that is greater than 1.0.

In some aspects, the lipolytic enzyme variants of the disclosure have an increased thermostability. In one aspect the lipolytic enzyme variants of the disclosure have an increased thermostability relative to a parent lipase, such as but not limited to the parent lipase shown in SEQ ID NO: 1 or 2.

As used herein “thermal stability” and “thermostability” and “thermostable” refer to lipolytic enzyme variants of the present disclosure that retain a specified amount of enzymatic activity after exposure to an identified temperature, often over a given period of time under conditions prevailing during the lipolytic, hydrolyzing, cleaning or other process disclosed herein, for example while exposed to altered temperatures. Altered temperatures include increased or decreased temperatures. In some embodiments, the lipolytic enzyme variant retains at least about 40%, about 50%, about 60%, about 70%, about 80%, about 85%, about 90%, about 92%, about 95%, about 96%, about 97%, about 98%, or about 99% lipolytic activity after exposure to altered temperatures over a given time period, for example, at least about 20 minutes, at least about 30 minutes, at least about 40 minutes, at least about 50 minutes, at least about 60 minutes, about 90 minutes, about 120 minutes, about 180 minutes, about 240 minutes, about 300 minutes, about 360 minutes, about 420 minutes, about 480 minutes, about 540 minutes, about 600 minutes, about 660 minutes, about 720 minutes, about 780 minutes, about 840 minutes, about 900 minutes, about 960 minutes, about 1020 minutes, about 1080 minutes, about 1140 minutes, or about 1200 minutes.

In one aspect the identified altered temperature is at least about 45° C.

In one aspect, the lipolytic enzyme variant or active fragment thereof of the disclosure, has a thermostability performance index (PI(thermostability)) relative to the parent lipolytic enzyme that is greater than 1.0.

In some aspects, the lipolytic enzyme variants of the disclosure have a decreased (reduced) malodor performance, where less malodor is produced relative to a reference polypeptide. In one aspect, the lipolytic enzyme variant or active fragment thereof of the disclosure, has a decreased malodor performance index (PI(malodor) relative to the lipolytic enzyme of SEQ ID NO:7 that is smaller than 1.0. As described herein, a malodor performance index (PI(malodor)) that is less than 1 (PI(malodor)<1) indicates improved performance by a variant as compared to the reference polypeptide, while a PI(malodor) of 1 (PI=1) identifies a variant that performs the same as the reference polypeptide, and a PI(malodor) that is greater than 1 (PI>1) identifies a variant that does not perform as well as the reference polypeptide.

In some aspects, the lipolytic enzyme variants of the disclosure have an increased calcium ion binding stability. In one aspect, the lipolytic enzyme variant or active fragment thereof of the disclosure, has a calcium ion binding stability index (PI(calcium ion binding stability)) relative to the parent lipolytic enzyme that is greater than 1.0.

An increased calcium ion binding stability may occur when the lipolytic enzyme retains the same level of activity when exposed to a reduced or low levels of Ca ion, when compared to a control. The term “reduced or low levels of Ca ion” means that the concentration of Ca ion in a solution has been reduced or lowered as compared to a control solution. Such reduced or low levels of Ca ion may be obtained by adding an agent that depletes a part or all Ca ion from the solution, including Ca ion that may be bound to the lipolytic enzyme present in the solution.

An increased calcium ion binding stability may occur when the lipolytic enzyme retains the same level of activity when exposed to EDTA (ethylenediaminetetraacetic acid), when compared to a control reaction without EDTA.

In some aspects, the lipolytic enzyme variants of the disclosure have a protease stability that is greater that the protease stability of the parent lipolytic enzyme.

In some embodiments, the lipolytic enzyme variant retains at least about 40%, about 50%, about 60%, about 70%, about 80%, about 85%, about 90%, about 92%, about 95%, about 96%, about 97%, about 98%, or about 99% lipolytic activity after exposure to proteolytic degradation over a given time period, for example, at least about 10 minutes, at least about 20 minutes, at least about 30 minutes, at least about 40 minutes, at least at least about 60 minutes, about 90 minutes, about 120 minutes, about 160 minutes, about 180 minutes, about 210 minutes, about 240 minutes, about 270 minutes, about 300 minutes, about 360 minutes, about 420 minutes, about 480 minutes, about 540 minutes or about 600 minutes.

In some aspects, an improved performance (improved properties) of a lipolytic enzyme variant includes a lipolytic enzyme variant with improved wash performance and an improved detergent stability, relative to the corresponding parent lipolytic enzyme.

In some aspects, an improved performance (improved properties) of a lipolytic enzyme variant includes a lipolytic enzyme variant with improved wash performance and an improved thermostability, relative to the corresponding parent lipolytic enzyme.

In some aspects, an improved performance (improved properties) of a lipolytic enzyme variant includes a lipolytic enzyme variant with improved wash performance and an improved calcium-ion binding stability, relative to the corresponding parent lipolytic enzyme.

In some aspects, an improved performance (improved properties) of a lipolytic enzyme variant includes a lipolytic enzyme variant with improved wash performance and an improved protease stability, relative to the corresponding parent lipolytic enzyme.

In some aspects, an improved performance (improved properties) of a lipolytic enzyme variant includes a lipolytic enzyme variant with improved detergent stability performance and an improved thermostability, relative to the corresponding parent lipolytic enzyme.

In some aspects, an improved performance (improved properties) of a lipolytic enzyme variant includes a lipolytic enzyme variant with improved detergent stability performance and an improved calcium-ion binding stability, relative to the corresponding parent lipolytic enzyme.

In some aspects, an improved performance (improved properties) of a lipolytic enzyme variant includes a lipolytic enzyme variant with improved detergent stability performance and an improved protease stability, relative to the corresponding parent lipolytic enzyme.

In some aspects, an improved performance (improved properties) of a lipolytic enzyme variant includes a lipolytic enzyme variant with improved thermostability performance and an improved calcium-ion binding stability, relative to the corresponding parent lipolytic enzyme.

In some aspects, an improved performance (improved properties) of a lipolytic enzyme variant includes a lipolytic enzyme variant with improved thermostability performance and an improved protease stability, relative to the corresponding parent lipolytic enzyme.

In some aspects, improved properties of a lipolytic enzyme variant includes a lipolytic enzyme variant with improved wash performance, improved thermostability and improved calcium ion binding stability, optionally with retained or improved stability and or protease stability, relative to the corresponding parent lipolytic enzyme.

In one embodiment, the disclosure provides a lipolytic enzyme variant or an active fragment thereof comprising an amino acid modification to a parent lipolytic enzyme, wherein the amino acid modification is at a position of the lipolytic enzyme variant selected from the group consisting of 2, 4, 11, 13, 14, 17, 18, 19, 20, 22, 27, 28, 29, 30, 31, 33, 43, 44, 46, 47, 49, 50, 52, 53, 57, 60, 62, 64, 65, 67, 68, 70, 83, 85, 91, 93, 95, 99, 105, 117, 118, 119, 120, 121, 122, 125, 127, 128, 130, 131, 132, 134, 135, 137, 138, 140, 141, 146, 147, 150, 154, 155, 159, 160, 164, 166, 170, 171, 175, 178, 179, 180, 185, 189, 196, 199, 203, 204, 205, 206, 208, 209, 212, 217, 218, 221, 223, 224, 227, 229, 247, 251, 252, 255, 258, 261, 262, 264, 267, 268, 272 and 280, wherein the amino acid positions of the variant are numbered by correspondence with the amino acid sequence of SEQ ID NO:2, wherein the lipolytic enzyme variant has at least 60% sequence identity to SEQ ID NO:1 or 2. In one aspect, the lipolytic enzyme variant is derived from a parent lipolytic enzyme having 60%, 65%, 70%, 75%, 80%, 85%, 86%, 87%, 88%, 89%, 90%, 91%, 92%, 93%, 94%, 95%, 96%, 97%, 98%, 99%, or 100% amino acid sequence identity to the amino acid sequence of SEQ ID NO: 1 or 2. In one aspect, the variant comprises an amino acid sequence having 60%, 65%, 70%, 75%, 80%, 85%, 86%, 87%, 88%, 89%, 90%, 91%, 92%, 93%, 94%, 95%, 96%, 97%, 98% or 99% amino acid sequence identity to the amino acid sequence of SEQ ID NO: 1 or 2.

In one embodiment, the disclosure provides a lipolytic enzyme variant or an active fragment thereof comprising an amino acid modification to a parent lipolytic enzyme, wherein the amino acid modification is a substitution selected from the group consisting of S002V, T4K, H011A, H011K, L013C, L013F, L013W, S014C, S014G, S014L, S014W, D017A, D017G, D017S, D017T, D018A, D018F, D018G, D018I, D018K, D018L, D018M, D018N, D018P, D018R, D018T, D018V, D018W, I019C, V020G, Y022F, Y022I, Y022V, Y022W, G027K, I028V, A29R, D030A, D030K, D030R, A031G, E033I, E033K, S043K, S043R, L044Q, A046G, A046S, A046T, F047C, F047Y, S049D, S049T, N050P, V052F, V052K, V052M, R053I, R053M, R053Q, R053T, R053V, L057C, F060K, Q062A, I64V, L065H, E067I, E067Q, E067R, E067S, E067T, T068S, A070V, A070Y, L083M, C085A, C085G, K091D, K091E, K091N, K091Q, A093E, S095P, S095N, V099I, V105A, V105C, V105P, R117A, R117C, R117D, R117E, R117G, R117I, R117K, R117N, R117Q, R117S, R117T, R117V, I118D, I118E, I118G, I118H, I118N, I118S, M119C, M119V, M119Y, R120A, R120C, R120E, R120F, R120G, R120H, R120I, R120K, R120L, R120M, R120Q, R120T, R120V, R120W, K121A, K121E, K121N, K121Q, K121S, K121T, D122P, P125D, P125E, P125F, Y127C, Y127D, Y127E, Y127G, Y127H, Y127N, I128A, I128C, I128G, I128K, I128M, I128N, I128Q, I128T, I128Y, A129S, D130C, A131D, A131E, A131N, A131P, A131Q, A131S, A131T, V132T, K134C, K134D, K134E, K134F, K134G, K134I, K134M, K134N, K134Q, K134S, K134T, K134V, K134W, K134Y, A135E, G137R, T138I, T138V, I140E, I140F, I140T, I140V, S141M, N146M, N146H, R147P, P150D, P150E, P150T, I154M, I154N, I154Q, I154T, I154V, A155E, A155M, A159D, A159S, L160A, L160C, L160S, L160T, L160V, N164A, N164E, N164Q, N164S, N164R, N164D, M166A, M166D, M166E, K170A, K170D, K170H, K170Q, K170T, K171C, K171D, K171E, K171Q, G175A, A178D, A178K, 1179E, I179K, R180E, R180P, K185C, K185D, K185E, K185L, K185S, N189D, F196I, Y199F, L203C, L203K, L203N, L203P, L203R, L203S, L203T, 1204Y, A205C, A205F, A205I, A205L, A205M, A205P, A205V, A205W, A205Y, G206C, G206D, G206E, G206Q, K208E, K208G, K208M, K208Q, G209A, G209C, G209D, G209E, G209Q, L212K, L212R, A217F, A217I, A217L, A217V, A217Y, A218C, V221F, V221T, S223G, A224G, A224H, A224K, A224Q, A224R, A224S, A224W, A224Y, S227D, S227I, S227N, S227T, S227V, R229A, R229C, R229D, R229E, R229G, R229H, R229I, R229K, R229L, R229M, R229N, R229P, R229Q, R229S, R229T, R229V, K247R, A251P, E252Q, L255C, V258I, V261L, A262C, A262F, A262L, A262M, L264K, R267P, G268D, G268K, I272G, I272K, I272P, I272R, I272V and N280K, wherein the amino acid positions of the variant are numbered by correspondence with the amino acid sequence of SEQ ID NO:2 wherein the lipolytic enzyme variant has at least 60%, 65%, 70%, 75%, 80%, 85%, 86%, 87%, 88%, 89%, 90%, 91%, 92%, 93%, 94%, 95%, 96%, 97%, 98%, 99%, or 100% amino acid sequence identity to SEQ ID NO: 1 or 2. In one embodiment, the disclosure provides a lipolytic enzyme variant or an active fragment thereof, wherein the variant or active fragment comprises amino acid modifications selected from the group consisting of D122P_A205P, T4K_L212R, T4K_L203R, I179K_L212R, A178K_G268K, N164R_A159D, N146H_I179K, N146H_N280K, G206D_G209A, G206E_G209D, G206E_G209A, A159D_G206E_G209D, T4K_L203R_L212R_G268K, A178K_I179K_L203R_L212R, N146H_A178K_I179K_G268K, N146H_A178K_I179K_L212R, T4K_N146H_I179K_L203R_L212R_N280K, T4K_N146H_A178K_L203R_G268K_N280K, A29R_I64V_V70A_S95N_S227T_I272V, A29R_I64V_V70A_S95N_S227T_R267P_I272V A29R_I64V_V70A_S95N_S227T_R267P_I272V, T4K_N146H_A159D_I179K_L203R_L212R_N280KT4K_N146H_I179K_L203R_G206E_G209D_L212R_N280K, and T4K_N146H_A159D_I179K_L203R_G206E_G209D_L212R_N280K, wherein the amino acid positions of the variant are numbered by correspondence with the amino acid sequence set forth in SEQ ID NO: 2, wherein the lipolytic enzyme variant has at least 60% sequence identity to SEQ ID NO: 1.

In one embodiment, the disclosure provides a lipolytic enzyme variant or an active fragment thereof, wherein the variant or active fragment comprises amino acid modifications selected from the group consisting of D122P_A205P, T4K_L212R, T4K_L203R, I179K_L212R, A178K_G268K, N164R_A159D, N146H_I179K, N146H_N280K, G206D_G209A, G206E_G209D, G206E_G209A, A159D_G206E_G209D, T4K_L203R_L212R_G268K, A178K_I179K_L203R_L212R, N146H_A178K_I179K_G268K, N146H_A178K_I179K_L212R, T4K_N146H_I179K_L203R_L212R_N280K, T4K_N146H_A178K_L203R_G268K_N280K, A29R_I64V_S95N_S227T_I272V, A29R_I64V_S95N_S227T_R267P_I272V A29R_I64V_S95N_S227T_R267P_I272V, T4K_N146H_A159D_I179K_L203R_L212R_N280K T4K_N146H_179K_L203R_G206E_G209D_L212R_N280K, and T4K_N146H_A159D_I179K_L203R_G206E_G209D_L212R_N280K, wherein the amino acid positions of the variant are numbered by correspondence with the amino acid sequence set forth in SEQ ID NO:2, wherein the lipolytic enzyme variant has at least 60% sequence identity to SEQ ID NO: 2.

### Polypeptides of the Disclosure

The present disclosure provides novel polypeptides, which may be collectively referred to as “polypeptides of the disclosure.” Polypeptides of the disclosure include isolated, recombinant, substantially pure, or non-naturally occurring lipolytic enzyme variant polypeptides, including for example, variant lipolytic enzyme polypeptides, having lipolytic activity and an improved property (performance) such as but not limiting to improved cleaning performance (such as improved wash performance), an increased expression, a decreased (reduced) malodor, an increased detergent stability, an increased thermostability, an increased calcium ion binding stability, an increased protease stability, or any one combination thereof.

In some embodiments, polypeptides of the disclosure are useful in cleaning applications and can be incorporated into cleaning compositions that are useful in methods of cleaning an item or a surface (e.g., a surface of an item) in need of cleaning.

In some embodiments, the disclosure includes an isolated, recombinant, substantially pure, or non-naturally occurring variant polypeptide having lipolytic activity, which polypeptide comprises a polypeptide sequence having at least having 60%, 65%, 70%, 75%, 80%, 85%, 86%, 87%, 88%, 89%, 90%, 91%, 92%, 93%, 94%, 95% 96%, 97%, 98%, 99%, or 100% amino acid sequence identity to the amino acid sequence of SEQ ID NO: 1 or 2.

In some embodiments, the variant polypeptide is a variant having a specified degree of amino acid sequence homology to the parent lipolytic enzyme, e.g., at least 60%, at least 65%, at least 70%, at least 75%, at least 80%, at least 85%, at least 90%, at least 91%, at least 92%, at least 93%, at least 94%, at least 95%, at least 96% at least 97%, at least 98%, or even at least 99% sequence homology to the amino acid sequence of SEQ ID NO: 1 or 2. Homology can be determined by amino acid sequence alignment, e.g., using a program such as BLAST, ALIGN, or CLUSTAL, as described herein.

In some embodiments, the variant polypeptide disclosed herein comprises an amino acid sequence having 60%, 65%, 70%, 75%, 80%, 85%, 86%, 87%, 88%, 89%, 90%, 91%, 92%, 93%, 94%, 95%, 96%, 97%, 98% or 99% amino acid sequence identity to the amino acid sequence of SEQ ID NO: 1 or 2.

Also provided is an isolated, recombinant, substantially pure, or non-naturally occurring sequence which encodes a lipolytic enzyme variant having lipolytic activity, said lipolytic enzyme variant (e.g., variant lipase) comprising an amino acid sequence which differs from the amino acid sequence of the parent lipase of SEQ ID NO:1 or 2 by no more than 50, no more than 40, no more than 30, no more than 35, no more than 25, no more than 20, no more than 19, no more than 18, no more than 17, no more than 16, no more than 15, no more than 14, no more than 13, no more than 12, no more than 11, no more than 10, no more than 9, no more than 8, no more than 7, no more than 6, no more than 5, no more than 4, no more than 3, no more than 2, or no more than 1 amino acid residue(s), wherein amino acid positions of the variant lipase are numbered according to the numbering of corresponding amino acid positions in the amino acid sequence of the parent lipolytic enzyme of SEQ ID NO: 1 or 2 as determined by alignment of the lipolytic enzyme variant amino acid sequence with the parent lipolytic amino acid sequence of SEQ ID NO: 1 or 2.

As noted above, the lipolytic enzyme variant polypeptides of the disclosure have enzymatic activities (e.g., lipolytic activities) and thus are useful in cleaning applications, including but not limited to, methods for cleaning dishware items, tableware items, fabrics, and items having hard surfaces (e.g., the hard surface of a table, table top, wall, furniture item, floor, ceiling, medical instrument, examination table, etc.). Exemplary cleaning compositions comprising one or more lipolytic enzyme variant polypeptides of the disclosure are described herein. The enzymatic activity (e.g., lipolytic enzyme activity) of a lipolytic enzyme variant polypeptide of the disclosure can be determined readily using procedures well known to those of ordinary skill in the art.

A polypeptide of the disclosure can be subject to various changes, such as one or more amino acid insertions, deletions, and/or substitutions, either conservative or non-conservative, including where such changes do not substantially alter the enzymatic activity of the polypeptide. Similarly, a nucleic acid of the disclosure can also be subject to various changes, such as one or more substitutions of one or more nucleic acids in one or more codons such that a particular codon encodes the same or a different amino acid, resulting in either a silent variation (e.g., mutation in a nucleotide sequence results in a silent mutation in the amino acid sequence, for example when the encoded amino acid is not altered by the nucleic acid mutation) or non-silent variation, one or more deletions of one or more nucleic acids (or codons) in the sequence, one or more additions or insertions of one or more nucleic acids (or codons) in the sequence, and/or cleavage of or one or more truncations of one or more nucleic acids (or codons) in the sequence. Many such changes in the nucleic acid sequence may not substantially alter the enzymatic activity of the resulting encoded lipolytic enzyme variant compared to the lipolytic enzyme variant encoded by the original nucleic acid sequence. A nucleic acid of the disclosure can also be modified to include one or more codons that provide for optimum expression in an expression system (e.g., bacterial expression system), while, if desired, said one or more codons still encode the same amino acid(s).

In some embodiments, the present disclosure provides a genus of polypeptides comprising lipolytic enzyme variant polypeptides having the desired activity or performance (e.g., lipolytic enzyme activity or cleaning performance) which comprise sequences having the amino acid substitutions described herein and also which comprise one or more additional amino acid substitutions, such as conservative and non-conservative substitutions, wherein the polypeptide exhibits, maintains, or approximately maintains the desired enzymatic activity (e.g., lipolytic enzyme activity or lipase activity, as reflected in the cleaning performance of the lipolytic enzyme variant). Amino acid substitutions in accordance with the disclosure may include, but are not limited to, one or more non-conservative substitutions and/or one or more conservative amino acid substitutions. A conservative amino acid residue substitution typically involves exchanging a member within one functional class of amino acid residues for a residue that belongs to the same functional class (identical amino acid residues are considered functionally homologous or conserved in calculating percent functional homology). A conservative amino acid substitution typically involves the substitution of an amino acid in an amino acid sequence with a functionally similar amino acid. For example, alanine, glycine, serine, and threonine are functionally similar and thus may serve as conservative amino acid substitutions for one another. Aspartic acid and glutamic acid may serve as conservative substitutions for one another. Asparagine and glutamine may serve as conservative substitutions for one another. Arginine, lysine, and histidine may serve as conservative substitutions for one another. Isoleucine, leucine, methionine, and valine may serve as conservative substitutions for one another. Phenylalanine, tyrosine, and tryptophan may serve as conservative substitutions for one another.

Other conservative amino acid substitution groups can be envisioned. For example, amino acids can be grouped by similar function or chemical structure or composition (e.g., acidic, basic, aliphatic, aromatic, sulfur-containing). For instance, an aliphatic grouping may comprise: Glycine (G), Alanine (A), Valine (V), Leucine (L), Isoleucine (I). Other groups containing amino acids that are considered conservative substitutions for one another include: aromatic: Phenylalanine (F), Tyrosine (Y), Tryptophan (W); sulfur-containing: Methionine (M), Cysteine (C); Basic: Arginine (R), Lysine (K), Histidine (H); Acidic: Aspartic acid (D), Glutamic acid (E); non-polar uncharged residues, Cysteine (C), Methionine (M), and Proline (P); hydrophilic uncharged residues: Serine (S), Threonine (T), Asparagine (N), and Glutamine (Q). Additional groupings of amino acids are well-known to those of skill in the art and described in various standard textbooks. Listing of a polypeptide sequence herein, in conjunction with the above substitution groups, provides an express listing of all conservatively substituted polypeptide sequences.

More conservative substitutions exist within the amino acid residue classes described above, which also or alternatively can be suitable. Conservation groups for substitutions that are more conservative include: valine-leucine-isoleucine, phenylalanine-tyrosine, lysine-arginine, alanine-valine, and asparagine-glutamine. Thus, for example, in some embodiments, the disclosure provides an isolated or recombinant lipolytic enzyme variant polypeptide (e.g., variant lipase) having lipolytic activity, said lipolytic enzyme variant polypeptide comprising an amino acid sequence having at least about 90%, about 95%, about 96%, about 97%, about 98%, about 99%, or about 99.5% sequence identity to the amino acid sequence of SEQ ID NO:1 or 2. A conservative substitution of one amino acid for another in a lipolytic enzyme variant of the disclosure is not expected to alter significantly the enzymatic activity or cleaning performance activity of the lipolytic enzyme variant. Enzymatic activity or cleaning performance activity of the resultant lipolytic enzyme can be readily determined using the standard assays and the assays described herein.

Conservatively substituted variations of a polypeptide sequence of the disclosure (e.g., lipolytic enzyme variants of the disclosure) include substitutions of a small percentage, sometimes less than about 25%, about 20%, about 15%, about 14%, about 13%, about 12%, about 11%, about 10%, about 9%, about 8%/6, about 7%, or about 6% of the amino acids of the polypeptide sequence, or less than about 5%, about 4%, about 3%, about 2%, or about 1%, of the amino acids of the polypeptide sequence, with a conservatively selected amino acid of the same conservative substitution group.

### Nucleic Acids of the Disclosure

The disclosure provides isolated, non-naturally occurring, or recombinant nucleic acids (also referred to herein as “polynucleotides”), which may be collectively referred to as “nucleic acids of the disclosure” or “polynucleotides of the disclosure”, which encode polypeptides of the disclosure. Nucleic acids of the disclosure, including all described below, are useful in recombinant production (e.g., expression) of polypeptides of the disclosure, typically through expression of a plasmid expression vector comprising a sequence encoding the polypeptide of interest or fragment thereof, or through chromosomal integration. In some aspects, a polynucleotide sequence encoding the lipolytic enzyme variant described herein (as well as other sequences included in the vector) is integrated into the genome of the host cell, while in other embodiments, a plasmid vector comprising a polynucleotide sequence encoding the lipolytic enzyme variant remains as autonomous extra-chromosomal element within the cell. As discussed above, polypeptides include isolated, recombinant, substantially pure, or non-naturally occurring lipolytic enzyme variant polypeptides, including for example, variant lipolytic enzyme polypeptides, having lipolytic activity and an improved property (performance) such as but not limiting to improved cleaning performance (such as improved wash performance), an increased expression, a decreased (reduced) malodor, an increased detergent stability, an increased thermostability, an increased calcium ion binding stability, an increased protease stability, or any one combination thereof, that are useful in cleaning applications and cleaning compositions for cleaning an item or a surface (e.g., surface of an item) in need of cleaning.

In one aspect, the polynucleotide of the disclosure is a DNA sequence of SEQ ID NO:3 or SEQ ID NO:4 encoding the lipolytic enzyme of SEQ ID NO: 1.

In some embodiments, the disclosure provides an isolated, recombinant, substantially pure, codon optimized, or non-naturally occurring nucleic acid comprising a nucleotide sequence encoding any polypeptide (including any fusion protein, etc.) of the disclosure described herein. The disclosure also provides an isolated, recombinant, substantially pure, or non-naturally-occurring nucleic acid comprising a nucleotide sequence encoding a combination of two or more of any polypeptides of the disclosure described above and elsewhere herein.

Also provided is an isolated, recombinant, substantially pure, or non-naturally occurring nucleic acid comprising a polynucleotide sequence which encodes a lipolytic enzyme variant having lipolytic activity, said lipolytic enzyme variant (e.g., variant lipase) comprising an amino acid sequence which differs from the amino acid sequence of SEQ ID NO:1 or 2 by no more than 50, no more than 40, no more than 30, no more than 35, no more than 25, no more than 20, no more than 19, no more than 18, no more than 17, no more than 16, no more than 15, no more than 14, no more than 13, no more than 12, no more than 11, no more than 10, no more than 9, no more than 8, no more than 7, no more than 6, no more than 5, no more than 4, no more than 3, no more than 2, or no more than 1 amino acid residue(s), wherein amino acid positions of the variant lipase are numbered according to the numbering of corresponding amino acid positions in the amino acid sequence of the lipolytic enzyme shown in SEQ ID NO:1 as determined by alignment of the lipolytic enzyme variant amino acid sequence with the parent lipolytic amino acid sequence of SEQ ID NO:1 or 2.

The present disclosure provides nucleic acids encoding a lipase variant of the parent lipase of SEQ ID NO:1 or 2, as described previously, wherein the amino acid positions of the lipase variant are numbered by correspondence with the parent lipolytic amino acid sequence of SEQ ID NO:1 or 2. The nucleic acids encoding the parent lipase or the lipase variants disclosed herein can be codon optimized for production in a host cell such as, but not limiting to Escherichia coli and Bacillus subtilus.

Nucleic acids of the disclosure can be generated by using any suitable synthesis, manipulation, and/or isolation techniques, or combinations thereof. For example, a polynucleotide of the disclosure may be produced using standard nucleic acid synthesis techniques, such as solid-phase synthesis techniques that are well-known to those skilled in the art. The synthesis of the nucleic acids of the disclosure can be also facilitated (or alternatively accomplished) by any suitable method known in the art, including but not limited to chemical synthesis using the classical phosphoramidite method (See e.g., Beaucage et al. Tetrahedron Letters 22:1859-69 (1981)); or the method described by Matthes et al. (See, Matthes et al., EMBO J. 3:801-805 (1984), as is typically practiced in automated synthetic methods. Nucleic acids of the disclosure also can be produced by using an automatic DNA synthesizer. Customized nucleic acids can be ordered from a variety of commercial sources (e.g., The Midland Certified Reagent Company, the Great American Gene Company, Operon Technologies Inc., and DNA20). Other techniques for synthesizing nucleic acids and related principles are known in the art (See e.g., Itakura et al., Ann. Rev. Biochem. 53:323 (1984); and Itakura et al., Science 198:1056 (1984)).

### Methods for Making Modified Lipolytic Enzyme Variants of the Disclosure

A variety of methods are known in the art that are suitable for generating modified polynucleotides of the disclosure that encode lipolytic enzyme variants of the disclosure, including, but not limited to, for example, site-saturation mutagenesis, scanning mutagenesis, insertional mutagenesis, deletion mutagenesis, random mutagenesis, site-directed mutagenesis, and directed-evolution, as well as various other recombinatorial approaches. Methods for making modified polynucleotides and proteins (e.g., lipolytic enzyme variants) include DNA shuffling methodologies, methods based on non-homologous recombination of genes, such as ITCHY (See, Ostermeier et al., 7:2139-44 (1999)), SCRACHY (See, Lutz et al. 98:11248-53 (2001)), SHIPREC (See, Sieber et al., 19:456-60 (2001)), and NRR (See, Bittker et al., 20:1024-9 (2001); Bittker et al., 101:7011-6 (2004)), and methods that rely on the use of oligonucleotides to insert random and targeted mutations, deletions and/or insertions (See, Ness et al., 20:1251-5 (2002); Coco et al., 20:1246-50 (2002); Zha et al., 4:34-9 (2003); Glaser et al., 149:3903-13 (1992)).

### Vectors, Cells, and Methods for Producing Lipolytic Enzyme Variants of the Disclosure

The present disclosure provides isolated or recombinant vectors comprising at least one polynucleotide of the disclosure described herein (e.g., a polynucleotide encoding a lipolytic enzyme variant of the disclosure described herein), isolated or recombinant expression vectors or expression cassettes comprising at least one nucleic acid or polynucleotide of the disclosure, isolated, substantially pure, or recombinant DNA constructs comprising at least one nucleic acid or polynucleotide of the disclosure, isolated or recombinant cells comprising at least one polynucleotide of the disclosure, cell cultures comprising cells comprising at least one polynucleotide of the disclosure, cell cultures comprising at least one nucleic acid or polynucleotide of the disclosure, and compositions comprising one or more such vectors, nucleic acids, expression vectors, expression cassettes, DNA constructs, cells, cell cultures, or any combination or mixtures thereof.

In one aspect, the recombinant DNA construct of the disclosure is a DNA sequence comprising SEQ ID NO:5 or 6.

In some embodiments, the disclosure provides recombinant cells comprising at least one vector (e.g., expression vector or DNA construct) of the disclosure which comprises at least one nucleic acid or polynucleotide of the disclosure. Some such recombinant cells are transformed or transfected with such at least one vector. Such cells are typically referred to as host cells. Some such cells comprise bacterial cells, including, but are not limited to Bacillus sp. cells, such as Bacillus subtilis cells. The disclosure also provides recombinant cells (e.g., recombinant host cells) comprising at least one lipolytic enzyme variant of the disclosure.

In some aspects, the lipolytic variants described herein are encoded by a DNA sequence that is codon optimized for expression in Escherichia coli. In some aspects, the lipolytic variants described herein are encoded by a DNA sequence that is codon optimized for expression in Bacillus subtilus.

In some embodiments, the disclosure provides a vector comprising a nucleic acid or polynucleotide of the disclosure. In some embodiments, the vector is an expression vector or expression cassette in which a polynucleotide sequence of the disclosure which encodes a lipolytic enzyme variant of the disclosure is operably linked to one or additional nucleic acid segments required for efficient gene expression (e.g., a promoter operably linked to the polynucleotide of the disclosure which encodes a lipolytic enzyme variant of the disclosure). A vector may include a transcription terminator and/or a selection gene, such as an antibiotic resistance gene that enables continuous cultural maintenance of plasmid-infected host cells by growth in antimicrobial-containing media.

An expression vector may be derived from plasmid or viral DNA, or in alternative embodiments, contains elements of both. Exemplary vectors include, but are not limited to pXX, pC194, pJH101, pE194, pHP13 (See, Harwood and Cutting [eds.], Chapter 3, Molecular Biological Methods for Bacillus, John Wiley & Sons [1990]; suitable replicating plasmids for B. subtilis include those listed on p. 92; See also, Perego, Integrational Vectors for Genetic Manipulations in Bacillus subtilis, in Sonenshein et al., [eds.] Bacillus subtilis and Other Gram-Positive Bacteria: Biochemistry, Physiology and Molecular Genetics, American Society for Microbiology, Washington, D.C. [1993], pp. 615-624.

For expression and production of a protein of interest (e.g., lipolytic enzyme variant) in a cell, at least one expression vector comprising at least one copy of a polynucleotide encoding the modified lipolytic enzyme, and preferably comprising multiple copies, is transformed into the cell under conditions suitable for expression of the lipolytic enzyme. In some embodiments of the present disclosure, a polynucleotide sequence encoding the lipolytic enzyme variant described herein (as well as other sequences included in the vector) is integrated into the genome of the host cell, while in other embodiments, a plasmid vector comprising a polynucleotide sequence encoding the lipolytic enzyme variant remains as autonomous extra-chromosomal element within the cell. The disclosure provides both extrachromosomal nucleic acid elements as well as incoming nucleotide sequences that are integrated into the host cell genome. The vectors described herein are useful for production of the lipolytic enzyme variants of the disclosure. In some embodiments, a polynucleotide construct encoding the lipolytic enzyme variant is present on an integrating vector that enables the integration and optionally the amplification of the polynucleotide encoding the lipolytic enzyme variant into the bacterial chromosome. Examples of sites for integration are well known to those skilled in the art. In some embodiments, transcription of a polynucleotide encoding a lipolytic enzyme variant of the disclosure is effectuated by a promoter that is the wild-type promoter for the selected precursor lipolytic enzyme. In some other embodiments, the promoter is heterologous to the precursor lipolytic enzyme, but is functional in the host cell. Specifically, examples of suitable promoters for use in microorganisms include, but are not limited to these described in WO2017/152169 (herein incorporated by reference) as well as promoters for use in bacterial host cells include, but are not limited to, for example, the amyE, amyQ, amyL, pstS, sacB, pSPAC, pAprE, pVeg, pHpaII promoters, the promoter of the B. stearothermophilus maltogenic amylase gene, the B. amyloliquefaciens (BAN) amylase gene, the B. subtilis alkaline lipolytic enzyme gene, the B. clausii alkaline lipolytic enzyme gene the B. pumilis xylosidase gene, the B. thuringiensis cryIIIA, and the B. licheniformis alpha-amylase gene. Additional promoters include, but are not limited to the A4 promoter, as well as phage Lambda PR or PL promoters, and the E. coli lac, trp or tac promoters.

Lipolytic enzyme variants of the present disclosure can be produced in any suitable host cells including but not limiting to suitable Gram-positive microorganism, bacteria and fungi. For example, in some embodiments, the lipolytic enzyme variant is produced in host cells of fungal and/or bacterial origin. In some embodiments, the host cells are Bacillus sp., Streptomyces sp., Escherichia sp. or Aspergillus sp. In some embodiments, the lipolytic enzyme variants are produced by Bacillus sp. host cells. Examples of Bacillus sp. host cells that find use in the production of the lipolytic enzyme variants of the disclosure include, but are not limited to B. licheniformis, B. lentus, B. subtilis, B. lentus, B. brevis, B. stearothermophilus, B. alkalophilus, B. coagulans, B. circulans, B. pumilis, B. thuringiensis, B. clausii, and B. megaterium, as well as other organisms within the genus Bacillus. In some embodiments, B. subtilis host cells are used for production of lipolytic enzyme variants. U.S. Pat. Nos. 5,264,366 and 4,760,025 (RE 34,606) describe various Bacillus host strains that can be used for producing lipolytic enzyme variants of the disclosure, although other suitable strains can be used.

Several industrial bacterial strains that can be used to produce lipolytic enzyme variants of the disclosure include non-recombinant (i.e., wild-type) Bacillus sp. strains, as well as variants of naturally-occurring strains and/or recombinant strains. In some embodiments, the host strain is a recombinant strain, wherein a polynucleotide encoding a polypeptide of interest has been introduced into the host. In some embodiments, the host strain is a B. subtilis host strain and particularly a recombinant Bacillus subtilis host strain. Numerous B. subtilis strains are known, including, but not limited to for example, 1A6 (ATCC 39085), 168 (1A01), SB19, W23, Ts85, B637, PB1753 through PB1758, PB3360, JH642, 1A243 (ATCC 39,087), ATCC 21332, ATCC 6051, MI113, DE100 (ATCC 39,094), GX4931, PBT 110, and PEP 211strain (See e.g., Hoch et al., Genetics 73:215-228 [1973]; See also, U.S. Pat. Nos. 4,450,235 and 4,302,544, and EP 0134048, each of which is incorporated by reference in its entirety). The use of B. subtilis as an expression host cells is well known in the art (See e.g., Palva et al., Gene 19:81-87 [1982]; Fahnestock and Fischer, J. Bacteriol., 165:796-804 [1986]; and Wang et al., Gene 69:39-47 [1988]).

In some embodiments, the Bacillus host cell is a Bacillus sp. that includes a mutation or deletion in at least one of the following genes, degU, degS, degR and degQ. Preferably the mutation is in a degU gene, and more preferably the mutation is degU(Hy)32 (See e.g., Msadek et al., J. Bacteriol. 172:824-834 [1990]; and Olmos et al., Mol. Gen. Genet. 253:562-567 [1997]). One suitable host strain is a Bacillus subtilis carrying a degU32(Hy) mutation. In some embodiments, the Bacillus host comprises a mutation or deletion in scoC4 (See e.g., Caldwell et al., J. Bacteriol. 183:7329-7340 [2001]); spoIIE (See e.g., Arigoni et al., Mol. Microbiol. 31:1407-1415 [1999]); and/or oppA or other genes of the opp operon (See e.g., Perego et al., Mol. Microbiol. 5:173-185 [1991]). Indeed, it is contemplated that any mutation in the opp operon that causes the same phenotype as a mutation in the oppA gene will find use in some embodiments of the altered Bacillus strain of the disclosure. In some embodiments, these mutations occur alone, while in other embodiments, combinations of mutations are present. In some embodiments, an altered Bacillus host cell strain that can be used to produce a lipolytic enzyme variant of the disclosure is a Bacillus host strain that already includes a mutation in one or more of the above-mentioned genes. In addition, Bacillus sp. host cells that comprise mutation(s) and/or deletions of endogenous lipolytic enzyme genes find use. In some embodiments, the Bacillus host cell comprises a deletion of the aprE and the nprE genes. In other embodiments, the Bacillus sp. host cell comprises a deletion of 5 lipolytic enzyme genes, while in other embodiments, the Bacillus sp. host cell comprises a deletion of 9 lipolytic enzyme genes (See e.g., U.S. Pat. Appln. Pub. No. 2005/0202535, incorporated herein by reference).

Host cells are transformed with at least one nucleic acid encoding at least one lipolytic enzyme variant of the disclosure using any suitable method known in the art. Whether the nucleic acid is incorporated into a vector or is used without the presence of plasmid DNA, it is typically introduced into a microorganism, in some embodiments, preferably an E. coli cell or a competent Bacillus cell. Methods for introducing a nucleic acid (e.g., DNA) into Bacillus cells or E. coli cells utilizing plasmid DNA constructs or vectors and transforming such plasmid DNA constructs or vectors into such cells are well known. In some embodiments, the plasmids are subsequently isolated from E. coli cells and transformed into Bacillus cells. However, it is not essential to use intervening microorganisms such as E. coli, and in some embodiments, a DNA construct or vector is directly introduced into a Bacillus host.

Those of skill in the art are well aware of suitable methods for introducing nucleic acid or polynucleotide sequences of the disclosure into Bacillus cells (See e.g., Ferrari et al., “Genetics,” in Harwood et al. [eds.], Bacillus, Plenum Publishing Corp. [1989], pp. 57-72; Saunders et al., J. Bacteriol. 157:718-726 [1984]; Hoch et al., J. Bacteriol. 93:1925-1937 [1967]; Mann et al., Current Microbiol. 13:131-135 [1986]; Holubova, Folia Microbiol. 30:97 [1985]; Chang et al., Mol. Gen. Genet. 168:11-115 [1979]; Vorobjeva et al., FEMS Microbiol. Lett. 7:261-263 [1980]; Smith et al., Appl. Env. Microbiol. 51:634 [1986]; Fisher et al., Arch. Microbiol. 139:213-217 [1981]; and McDonald, J. Gen. Microbiol. 130:203 [1984]). Indeed, such methods as transformation, including protoplast transformation and congression, transduction, and protoplast fusion are well known and suited for use in the present disclosure. Methods of transformation are used to introduce a DNA construct or vector comprising a nucleic acid encoding a lipolytic enzyme variant of the present disclosure into a host cell. Methods known in the art to transform Bacillus cells include such methods as plasmid marker rescue transformation, which involves the uptake of a donor plasmid by competent cells carrying a partially homologous resident plasmid (See, Contente et al., Plasmid 2:555-571 [1979]; Haima et al., Mol. Gen. Genet. 223:185-191 [1990]; Weinrauch et al., J. Bacteriol. 154:1077-1087 [1983]; and Weinrauch et al., J. Bacteriol. 169:1205-1211 [1987]). In this method, the incoming donor plasmid recombines with the homologous region of the resident “helper” plasmid in a process that mimics chromosomal transformation.

In addition to commonly used methods, in some embodiments, host cells are directly transformed with a DNA construct or vector comprising a nucleic acid encoding a lipolytic enzyme variant of the disclosure (i.e., an intermediate cell is not used to amplify, or otherwise process, the DNA construct or vector prior to introduction into the host cell). Introduction of the DNA construct or vector of the disclosure into the host cell includes those physical and chemical methods known in the art to introduce a nucleic acid sequence (e.g., DNA sequence) into a host cell without insertion into a plasmid or vector. Such methods include, but are not limited to calcium chloride precipitation, electroporation, naked DNA, liposomes and the like. In additional embodiments, DNA constructs or vector are co-transformed with a plasmid, without being inserted into the plasmid. In further embodiments, a selective marker is deleted from the altered Bacillus strain by methods known in the art (See, Stahl et al., J. Bacteriol. 158:411-418 [1984]; and Palmeros et al., Gene 247:255-264 [2000]).

In some embodiments, the transformed cells of the present disclosure are cultured in conventional nutrient media. The suitable specific culture conditions, such as temperature, pH and the like are known to those skilled in the art and are well described in the scientific literature. In some embodiments, the disclosure provides a culture (e.g., cell culture) comprising at least one lipolytic enzyme variant or at least one nucleic acid of the disclosure. Also provided are compositions comprising at least one nucleic acid, vector, or DNA construct of the disclosure.

In some embodiments, host cells transformed with at least one polynucleotide sequence encoding at least one lipolytic enzyme variant of the disclosure are cultured in a suitable nutrient medium under conditions permitting the expression of the present lipolytic enzyme, after which the resulting lipolytic enzyme is recovered from the culture. The medium used to culture the cells comprises any conventional medium suitable for growing the host cells, such as minimal or complex media containing appropriate supplements. Suitable media are available from commercial suppliers or may be prepared according to published recipes (See e.g., the catalogues of the American Type Culture Collection). In some embodiments, the lipolytic enzyme produced by the cells is recovered from the culture medium by conventional procedures, including, but not limited to for example, separating the host cells from the medium by centrifugation or filtration, precipitating the proteinaceous components of the supernatant or filtrate by means of a salt (e.g., ammonium sulfate), chromatographic purification (e.g., ion exchange, gel filtration, affinity, etc.). Any method suitable for recovering or purifying a lipolytic enzyme variant finds use in the present disclosure.

In some embodiments, a lipolytic enzyme variant produced by a recombinant host cell is secreted into the culture medium. A nucleic acid sequence that encodes a purification facilitating domain may be used to facilitate purification of soluble proteins. A vector or DNA construct comprising a polynucleotide sequence encoding a lipolytic enzyme variant may further comprise a nucleic acid sequence encoding a purification facilitating domain to facilitate purification of the lipolytic enzyme variant (See e.g., Kroll et al., DNA Cell Biol. 12:441-53 [1993]). Such purification facilitating domains include, but are not limited to, for example, metal chelating peptides such as histidine-tryptophan modules that allow purification on immobilized metals (See, Porath, Protein Expr. Purif. 3:263-281 [1992]), protein A domain that allow purification on immobilized immunoglobulin, and the domain utilized in the FLAGS extension/affinity purification system (e.g., protein A domains available from Immunex Corp., Seattle, WA). The inclusion of a cleavable linker sequence such as Factor XA or enterokinase (e.g., sequences available from Invitrogen, San Diego, CA) between the purification domain and the heterologous protein also find use to facilitate purification.

Assays for detecting and measuring the enzymatic activity of an enzyme, such as a lipolytic enzyme variant of the disclosure, are well known. Various assays for detecting and measuring activity of lipolytic enzymes (e.g., lipolytic enzyme variants of the disclosure), are also known to those of ordinary skill in the art. A variety of methods can be used to determine the level of production of a mature lipolytic enzyme (e.g., mature lipolytic enzyme variants of the present disclosure) in a host cell. Such methods include, but are not limited to, methods that utilize either polyclonal or monoclonal antibodies specific for the lipolytic enzyme. Exemplary methods include, but are not limited to enzyme-linked immunosorbent assays (ELISA), radioimmunoassays (RIA), fluorescent immunoassays (FIA), and fluorescent activated cell sorting (FACS). These and other assays are well known in the art (See e.g., Maddox et al., J. Exp. Med. 158:1211 [1983]).

In some other embodiments, the disclosure provides methods for making or producing a mature lipolytic enzyme variant of the disclosure. A mature lipolytic enzyme variant does not include a signal peptide or a propeptide sequence. Some methods comprise making or producing a lipolytic enzyme variant of the disclosure in a recombinant bacterial host cell, such as for example, a Bacillus sp. cell (e.g., a B. subtilis cell). In some embodiments, the disclosure provides a method of producing a lipolytic enzyme variant of the disclosure, the method comprising cultivating a recombinant host cell comprising a recombinant expression vector comprising a nucleic acid encoding a lipolytic enzyme variant of the disclosure under conditions conducive to the production of the lipolytic enzyme variant. Some such methods further comprise recovering the lipolytic enzyme variant from the culture.

In some embodiments the disclosure provides methods of producing a lipolytic enzyme variant of the disclosure, the methods comprising: (a) introducing a recombinant expression vector comprising a nucleic acid encoding a lipolytic enzyme variant of the disclosure into a population of cells (e.g., bacterial cells, such as B. subtilis cells); and (b) culturing the cells in a culture medium under conditions conducive to produce the lipolytic enzyme variant encoded by the expression vector. Some such methods further comprise: (c) isolating the lipolytic enzyme variant from the cells or from the culture medium.

### Compositions Comprising Lipolytic Enzyme Variants of the Disclosure

Some further embodiments are directed to compositions comprising one or more lipolytic enzyme variants described herein, such as but not limited to cleaning compositions.

Cleaning compositions and cleaning formulations include any composition that is suited for cleaning, bleaching, disinfecting, and/or sterilizing any object, item, and/or surface. Such compositions and formulations include, but are not limited to for example, liquid and/or solid compositions, including cleaning or detergent compositions (e.g., liquid, tablet, gel, bar, granule, pouch, and/or solid laundry cleaning or detergent compositions and fine fabric detergent compositions; hard surface (such as but not limiting to the hard surface of a table, table top, wall, furniture item, floor, ceiling, medical instrument, examination table, etc.) cleaning compositions and formulations, such as for glass, wood, ceramic and metal counter tops and windows; carpet cleaners; oven cleaners; fabric fresheners; fabric softeners; and textile, laundry booster cleaning or detergent compositions, laundry additive cleaning compositions, and laundry pre-spotter cleaning compositions; dishwashing compositions, including hand or manual dishwash compositions (e.g., “hand” or “manual” dishwashing detergents) and automatic dishwashing compositions (e.g., “automatic dishwashing detergents”).

Cleaning composition or cleaning formulations, as used herein, include, unless otherwise indicated, granular or powder-form all-purpose or heavy-duty washing agents, especially cleaning detergents; liquid, granular, gel, solid, tablet, or paste-form all-purpose washing agents, especially the so-called heavy-duty liquid (HDL) detergent or heavy-duty powder detergent (HDD) types; liquid fine-fabric detergents; hand or manual dishwashing agents, including those of the high-foaming type; hand or manual dishwashing, automatic dishwashing, or dishware or tableware washing agents, including the various tablet, powder, solid, granular, liquid, gel, and rinse-aid types for household and institutional use; liquid cleaning and disinfecting agents, including antibacterial hand-wash types, cleaning bars, mouthwashes, denture cleaners, car shampoos, carpet shampoos, bathroom cleaners; hair shampoos and/or hair-rinses for humans and other animals; shower gels and foam baths and metal cleaners; as well as cleaning auxiliaries, such as bleach additives and “stain-stick” or pre-treat types. In some embodiments, granular compositions are in “compact” form; in some embodiments, liquid compositions are in a “concentrated” form.

In some embodiments, the cleaning compositions of the present disclosure are provided in unit dose form, including tablets, capsules, sachets, pouches, and multi-compartment pouches. In some embodiments, the unit dose format is designed to provide controlled release of the ingredients within a multi-compartment pouch (or other unit dose format). Suitable unit dose and controlled release formats are known in the art (See e.g., EP 2 100 949, WO 02/102955, U.S. Pat. Nos. 4,765,916 and 4,972,017, and WO 04/111178 for materials suitable for use in unit dose and controlled release formats). In some embodiments, the unit dose form is provided by tablets wrapped with a water-soluble film or water-soluble pouches. Various formats for unit doses are provided in EP 2 100 947, and are known in the art.

In some embodiments of the present disclosure, the cleaning compositions comprise at least one lipolytic enzyme variant of the present disclosure at a level from about 0.00001% to about 10% of enzyme protein by weight of the composition and the balance (e.g., about 99.999% to about 90.0%) comprising cleaning adjunct materials by weight of composition. In some embodiments of the present disclosure, the cleaning compositions of the present disclosure comprises at least one lipolytic enzyme variant at a level of about 0.00001% to about 10%, about 0.00001% to about 5%, about 0.0001% to 1%, about 0.001% to 0.5%, about 0.001% to about 2%, about 0.005% to about 0.5%, about 0.01% to 0.2% of enzyme protein by weight of the composition and the balance of the cleaning composition (e.g., about 99.9999% to about 90.0%, about 99.999% to about 98%, about 99.995% to about 99.5% by weight) comprising cleaning adjunct materials.

In some embodiments, the cleaning composition is a granular or powder laundry detergent. In some embodiments, the cleaning composition is a liquid laundry detergent or a dish washing detergent.

As used herein, the term “detergent composition” or “detergent formulation” is used in reference to a composition intended for use in a wash medium for the cleaning of soiled or dirty objects, including particular fabric and/or non-fabric objects or items. Such compositions of the present disclosure are not limited to any particular detergent composition or formulation. Indeed, in some embodiments, the detergents of the disclosure comprise at least one lipolytic enzyme variant of the disclosure and, in addition, one or more surfactants, transferase(s), hydrolytic enzymes, oxido reductases, builders (e.g., a builder salt), bleaching agents, bleach activators, bluing agents, fluorescent dyes, caking inhibitors, masking agents, enzyme activators, antioxidants, and/or solubilizers. In some instances, a builder salt is a mixture of a silicate salt and a phosphate salt, preferably with more silicate (e.g., sodium metasilicate) than phosphate (e.g., sodium tripolyphosphate). Some compositions of the disclosure, such as, but not limited to, cleaning compositions or detergent compositions, do not contain any phosphate (e.g., phosphate salt or phosphate builder).

Unless otherwise noted, all component or composition levels provided herein are made in reference to the active level of that component or composition, and are exclusive of impurities, for example, residual solvents or by-products, which may be present in commercially available sources. Enzyme components weights are based on total active protein. All percentages and ratios are calculated by weight unless otherwise indicated. All percentages and ratios are calculated based on the total composition unless otherwise indicated. In the exemplified detergent compositions, the enzymes levels are expressed by pure enzyme by weight of the total composition and unless otherwise specified, the detergent ingredients are expressed by weight of the total compositions.

In some embodiments, the lipolytic enzyme variants of the present disclosure can be used in compositions comprising an adjunct material and a lipolytic enzyme variant, wherein the composition is cleaning composition.

In some embodiments, the cleaning compositions of the present disclosure comprises one or more lipolytic enzymes and adjunct materials. In some embodiments, these adjuncts are incorporated for example, to assist or enhance cleaning performance, for treatment of the substrate to be cleaned, or to modify the aesthetics of the cleaning composition as is the case with perfumes, colorants, dyes or the like. It is understood that such adjuncts are in addition to the lipolytic enzyme variants of the present disclosure. The precise nature of these additional components, and levels of incorporation thereof, will depend on the physical form of the composition and the nature of the cleaning operation for which it is to be used. Suitable adjunct materials include, but are not limited to, surfactants (for example, surfactants that are efficient in removal of fatty acids from the fabric), builders, chelating agents, dye transfer inhibiting agents, dye transfer agents, deposition aids, dispersants, additional enzymes, and enzyme stabilizers, catalytic materials, bleaching agents, bleach activators, bleach catalysts, bleach boosters, hydrogen peroxide, sources of hydrogen peroxide, preformed peracids, polymeric dispersing agents, clay soil removal/anti-redeposition agents, brighteners, optical brighteners, suds suppressors, dyes, perfumes, structure elasticizing agents, fabric conditioners, fabric softeners, carriers, hydrotropes, processing aids and/or pigments, preservatives, anti-oxidants, anti-shrinkage agents, anti-wrinkle agents, germicides, fungicides, filler salts, hydrotropes, photoactivators, fluorescers, colorants, color speckles, silver care, anti-tarnish and/or anti-corrosion agents, alkalinity sources, solubilizing agents, carriers, processing aids, pigments, soil release polymers, dispersants, other enzymes, enzyme stabilizing systems and pH control agents. In addition to the disclosure below, suitable examples of such other adjuncts and levels of use are found in U.S. Pat. Nos. 5,576,282, 6,306,812, and 6,326,348, incorporated by reference. The aforementioned adjunct ingredients may constitute the balance of the cleaning compositions of the present disclosure.

In embodiments in which the cleaning adjunct materials are not compatible with the lipolytic enzyme variants of the present disclosure in the cleaning compositions, then suitable methods of keeping the cleaning adjunct materials and the lipolytic enzyme(s) separated (i.e., not in contact with each other) until combination of the two components is appropriate are used. Such separation methods include any suitable method known in the art (e.g., gelcaps, encapsulation, tablets, physical separation, etc.).

In some embodiments, the adjuvant and lipolytic enzyme are present in a single composition. In other embodiments, the adjuvant and lipolytic enzyme are present in separate compositions that are combined before contacting an oil stain on fabric, or combined on the oil stain.

The present cleaning compositions can include one or more adjuvants for use in combination with a lypolytic enzyme. Suitable adjuvants can have a relatively small hydrophilic portion with no net charge and hydrophobic portion that is linear or saturated. In some embodiments, the hydrophobic portion includes at least, six, seven, eight, or nine adjacent aliphatic carbons. In some embodiments, the hydrophobic portion is cyclic. In some embodiments, the hydrophobic portion is not branched. Suitable adjuvants include surfactants including sugar-based compounds and zwitterionic compounds. Suitable adjuvants are disclosed, and hereby incorporated by reference in its entirety, in WO2011078949.

Sugar-based surfactants include maltopyranosides, thiomaltopyransodies, glucopyranosides, and their derivatives. Maltose-based surfactants were generally more effective than glucose-based surfactants. In some embodiments, a preferred sugar-based surfactant has a hydrophobic tail chain length of at least 4, at least 5, at least 6, and even at least 7 carbons. The tail can be aliphatic or cyclic. The tail can be unbranched, although branching is acceptable with sufficient chain length.

Particular examples of sugar-based surfactants include nonyl-β-D-maltopyranoside, decyl-β-D-maltopyranoside, undecyl-β-D-maltopyranoside, dodecyl-β-D-maltopyranoside, tridecyl-β-D-maltopyranoside, tetradecyl-β-D-maltopyranoside, hexaecyl-β-D-maltopyranoside, n-dodecyl-β-D-maltopyranoside and the like, 2,6-dimethyl-4-heptyl-β-D-maltopyranoside, 2-propyl-1-pentyl-β-D-maltopyranoside, nonyl-β-D-glucopyranoside, nonyl-β-D-glucopyranoside, decyl-β-D-glucopyranoside, dodecyl-β-D-glucopyranoside, sucrose monododecanoate, certain cyclohexylalkyl-β-D-maltosides (e.g., the CYMAL®s and CYGLAs), and the MEGA™ surfactants.

The adjuvant can be a non-sugar, non-ionic surfactant. Exemplary surfactants include alkyl ethoxylates and alkylphenol ethoxylates, Tritons with an ethoxylate repeat of nine or less. Particular Tritons are ANAPOE®-X-100 and ANAPOE®-X-114. In some embodiments, the adjuvant is a non-ionic phosphine oxide surfactant, having a hydrophobic tail of at least about 9 carbons. Exemplary surfactants include dimethyldecylphoshine oxide and dimethyldodecylphoshine oxide.

The adjuvant can be a zwitterionic surfactant, such as a FOS-choline. In some embodiments, the FOS-choline has a hydrophobic tail with a chain length of 12 or greater. The hydrophobic tail can be saturated and unsaturated and can be cyclic. Exemplary FOS-choline surfactants include FOS-CHOLINE®-12, FOS-CHOLINE®-13, FOS-CHOLINE®-14, LYSOFOS-CHOLINE®-14, FOS-CHOLINE®-15, FOS-CHOLINE®-16, FOS-MEA®-12, DODECAFOS, ISO unsat 11-10, ISO 11-6, CYOFO, NOPOL-FOS, CYCLOFOS® (CYMAL®)-5, -6. -7, -8, etc., and the like.

In some cases, the adjuvant can be a sulfobetaine zwitterionic surfactant. Preferred sulfobetaine surfactants have a hydrophobic tail having at least 12 carbons, e.g., ANZERGENT® 3-12 and ANZERGENT® 3-14. The zwitterionic oxides and CHAPS-based surfactants (e.g. CHAPS and CHAPSO) are also effective, typically at higher doses than the sulfobetaines.

In some cases, the adjuvant can be an anionic detergent, for example, a sarcosine. Preferred sarcosines have a hydrophobic tail having at least 10 carbons. In some cases, the adjuvant can also be deoxycholate.

In some embodiments, the cleaning compositions comprising at least one lipolytic enzyme variant comprise one or more of the following ingredients (based on total composition weight): from about 0.0005 wt % to about 0.5 wt %, from about 0.001 wt % to about 0.1 wt %, or even from about 0.002 wt % to about 0.05 wt % of said lipolytic enzyme variant; and one or more of the following: from about 0.00003 wt % to about 0.1 wt % fabric hueing agent; from about 0.001 wt % to about 5 wt %, perfume capsules; from about 0.001 wt % to about 1 wt %, cold-water soluble brighteners; from about 0.00003 wt % to about 0.1 wt % bleach catalysts; from about 0.00003 wt % to about 0.1 wt % bacterial cleaning cellulases; and/or from about 0.05 wt % to about 20 wt % Guerbet nonionic surfactants.

The adjuvant can be present in a composition in an amount of at least 0.001%, at least 0.005%, at least 0.01%, at least 0.05%, at least 0.1%, or more, or at least 0.01 ppm, at least 0.05 ppm, at least 0.1 ppm, at least 0.5 ppm, at least 1 ppm, at least 5 ppm, at least 10 ppm, or more. In some cases, the adjuvant may be present in a preselected range, e.g., about 0.001-0.01%, about 0.01-0.1%, about 0.1-1%, or about 0.01-1 ppm, about 0.1-1 ppm, or about 1-10 ppm. In some cases, optimum activity is observed over a range, above and below which activity is reduced.

The surfactant system of the detergent can comprise nonionic, anionic, cationic, ampholytic, and/or zwitterionic surfactants. The surfactant is typically present at a level from 0.1% to 60% by weight, e.g. 1% to 40%, particularly 10-40% preferably from about 3% to about 20% by weight. The detergent will usually contain 0-50% of anionic surfactant such as linear alkylbenzenesulfonate (LAS), alpha-olefin sulfonate (AOS), alkyl sulfate (fatty alcohol sulfate) (AS), alcohol ethoxysulfate (AEOS or AES), secondary alkane sulfonates (SAS), alpha-sulfo fatty acid methyl esters, alkyl- or alkenylsuccinic acid or soap.

The detergent can comprise 0-40% of nonionic surfactant polyalkylene oxide (e.g. polyethylene oxide) condensates of alkyl phenols. Preferred nonionic surfactants are alcohol ethoxylate (AEO or AE), carboxylated alcohol ethoxylates, nonylphenol ethoxylate, alkylpolyglycoside, alkyldimethylamineoxide, ethoxylated fatty acid monoethanolamide, fatty acid monoethanolamide, alkyl(N-methyl)-glucoseamide or polyhydroxy alkyl fatty acid amide (e.g. as described in WO 92106154).

Semi-polar nonionic surfactants are another category of nonionic surfactants which include water-soluble amine oxides containing one alkyl moiety of from about 10 to about 18 carbon atoms and 2 moieties selected from the group consisting of alkyl groups and hydroxyalkyl groups containing from about 1 to about 3 carbon atoms; water soluble phosphine oxides containing one alkyl moiety of from about 10 to about 18 carbon atoms and 2 moieties selected from the group consisting of alkyl groups and hydroxyalkyl groups containing from about 1 to about 3 carbon atoms; and water-soluble sulfoxides containing one alkyl moiety from about 10 no to about 18 carbon atoms and a moiety selected from the group consisting of alkyl and hydroxyalkyl moieties of from about 1 to about 3 carbon atoms. The amine oxide surfactants in particular include C₁₀-C₁₈ alkyl dimethyl amine oxides and C₈-C₁₂ alkoxy ethyl dihydroxy ethyl amine oxides.

The detergent composition can further comprise cationic surfactants. Cationic detersive surfactants used are those having one long-chain hydrocarbyl group. Examples of such cationic surfactants include the ammonium surfactants such as alkyl trimethyl ammonium halogenides. Highly preferred cationic surfactants are the water soluble quaternary ammonium compounds. Examples of suitable quaternary ammonium compounds include coconut trimethyl ammonium chloride or bromide; coconut methyl dihydroxy ethyl ammonium chloride or bromide; decyl triethyl ammonium chloride; decyl dimethyl hydroxyl ethyl ammonium chloride or bromide; C12-15 dimethyl hydroxyl ethyl ammonium chloride or bromide; coconut dimethyl hydroxyl ethyl ammonium chloride or bromide; myristyl trimethyl ammonium methyl sulphate; lauryl dimethyl benzyl ammonium chloride or bromide; lauryl dimethyl (ethenoxy)₄ ammonium chloride or bromide; choline esters, dialkyl imidazolines.

The detergent composition can further comprise ampholytic surfactants. These surfactants can be broadly described as aliphatic derivatives of secondary or tertiary amines, or aliphatic derivatives of heterocyclic secondary and tertiary amines in which the aliphatic radical can be straight-, or branched-chain. One of the aliphatic substituent contains at least about 8 carbon atoms, typically from about 8 to about 18 carbon atoms, and at least one contains an anionic water-solubilizing group, e.g. carboxy, sulfonate, sulfate. Examples of compounds falling within this definition are sodium 3-(dodecylamino) propionate, sodium 3-(dodecylamino)-propane-1-sulfonate, sodium 2-(dodecylamino)ethyl sulfate, sodium 2-(dimethylamino)octadecanoate, di-sodium 3-(N-carboxymethyldodecylamino)propane-I-sulfonate, disodium octadecyl-iminodiacetate, sodium 1-carboxymethyl-2-undecylimidazole, and sodium N,N-bis(2-hydroxyethyl)-2-sulfato-3-dodecoxy-propylamine. Sodium 3-(dodecylamino )propane-I-sulfonate is preferred.

Zwitterionic surfactants are also used in detergent compositions especially within laundry. These surfactants can be broadly described as derivatives of secondary and tertiary amines, derivatives of heterocyclic secondary and tertiary amines, or derivatives of quaternary ammonium, quaternary phosphonium or tertiary sulfonium compounds. The cationic atom in the quaternary compound can be part of a heterocyclic ring. In all of these compounds, there is at least one aliphatic group, straight chain or branched, containing from about 3 to 18 carbon atoms and at least one aliphatic substituent containing an anionic water solubilizing group, e.g. carboxy, sulfonate, sulfate, phosphate or phosphonate. Ethoxylated zwitterionic compounds in combination with zwitterionic surfactants have been particularly used for clay soil removal in laundry applications.

In some embodiments in which the cleaning compositions of the present disclosure are formulated as compositions suitable for use in laundry machine washing method(s), the compositions of the present disclosure preferably contain at least one surfactant and at least one builder compound, as well as one or more cleaning adjunct materials preferably selected from organic polymeric compounds, bleaching agents, additional enzymes, suds suppressors, dispersants, lime-soap dispersants, soil suspension and anti-redeposition agents and corrosion inhibitors. In some embodiments, laundry compositions also contain softening agents (i.e., as additional cleaning adjunct materials).

The detergent may contain 1-65% of a detergent builder or complexing agent such as zeolite, diphosphate, triphosphate, phosphonate, citrate, nitrilotriacetic acid (NTA), ethylenediaminetetraacetic acid (EDTA), diethylenetriaminepentaacetic acid (DTMPA), alkyl- or alkenylsuccinic acid, soluble silicates or layered silicates (e.g. SKS-6 from Hoechst). The detergent may also be unbuilt i.e. essentially free of detergent builder.

The detergent builders may be subdivided into phosphorus-containing and non-phosphorous-containing types. Examples of phosphorus-containing inorganic alkaline detergent builders include the water-soluble salts, especially alkali metal pyrophosphates, orthophosphates, polyphosphates and phosphonates. Examples of non-phosphorus-containing inorganic builders include water soluble alkali metal carbonates, borates and silicates as well as layered disilicates and the various types of water insoluble crystalline or amorphous alumino silicates of which zeolites is the best known representative. Examples of suitable organic builders include alkali metal, ammonium or substituted ammonium salts of succinates, malonates, fatty acid malonates, fatty acid sulphonates, carboxymethoxy succinates, poly acetates, carboxylates, polycarboxylates, aminopolycarboxylates and polyacetyl carboxylates.

A suitable chelant for inclusion in the detergent compositions is ethylenediamine-N,N-disuccinic acid (EDDS) or the alkali metal, alkaline earth metal, ammonium, or substituted ammonium salts thereof or mixtures thereof. Some EDDS compounds are the free acid form and the sodium or magnesium salt thereof. Examples of such sodium salts of EDDS include Na2EDDS and Na4EDDS. Examples of such magnesium salts of EDDS include MgEDDS and Mg2EDDS.

The detergent may comprise one or more polymers. Examples are carboxymethylcellulose (CMC), poly (vinylpyrrolidone) (PVP), polyethyleneglycol (PEG), poly (vinyl alcohol) (PVA), polycarboxylates such as polyacrylates, maleic/acrylic acid copolymers and lauryl methacrylate/acrylic acid copolymers.

The detergent composition may contain bleaching agents of the chlorine/bromine-type or the oxygen-type. The bleaching agents may be coated or encapsulated. Examples of inorganic chlorine/bromine-type bleaches are lithium, sodium or calcium hypochlorite or hypobromite as well as chlorinated trisodium phosphate. The bleaching system may also comprise a hydrogen peroxide source such as perborate or percarbonate which may be combined with a peracid-forming bleach activator such as tetraacetyl-ethylenediamine (TAED) or nonanoyloxybenzenesulfonate (NOBS). Examples of organic chlorine/bromine-type bleaches are heterocyclic N-bromo and N-chloro imides such as trichloroisocyanuric, tribromoisocyanuric, dibromoisocyanuric and dichloroisocyanuric acids, and salts thereof with water solubilizing cations such as potassium and sodium. Hydantoin compounds are also suitable. The bleaching system may also comprise peroxyacids of, e.g., the amide, imide, or sulfone type.

In dishwashing detergents, the oxygen bleaches are preferred, for example in the form of an inorganic persalt, preferably with a bleach precursor or as a peroxy acid compound. Typical examples of suitable peroxy bleach compounds are alkali metal perborates, both tetrahydrates and monohydrates, alkali metal percarbonates, persilicates and perphosphates. Preferred activator materials are tetraacetylethylenediamine (TAED), nonanoyloxybenzenesulfonate (NOBS), 3,5-trimethyl-hexsanoloxybenzenesulfonate (ISONOBS) or pentaacetylglucose (PAG).

In some embodiments, bleaches, bleach activators and/or bleach catalysts are present in the compositions of the present disclosure. In some embodiments, the cleaning compositions of the present disclosure comprise inorganic and/or organic bleaching compound(s). Inorganic bleaches include, but are not limited to perhydrate salts (e.g., perborate, percarbonate, perphosphate, persulfate, and persilicate salts). In some embodiments, inorganic perhydrate salts are alkali metal salts. In some embodiments, inorganic perhydrate salts are included as the crystalline solid, without additional protection, although in some other embodiments, the salt is coated. Any suitable salt known in the art finds use in the present disclosure (See e.g., EP 2 100 949).

In some embodiments, bleach activators are used in the compositions of the present disclosure. Bleach activators are typically organic peracid precursors that enhance the bleaching action in the course of cleaning at temperatures of 60° C. and below. Bleach activators suitable for use herein include compounds which, under perhydrolysis conditions, give aliphatic peroxoycarboxylic acids having preferably from about 1 to about 10 carbon atoms, in particular from about 2 to about 4 carbon atoms, and/or optionally substituted perbenzoic acid. Additional bleach activators are known in the art and find use in the present disclosure (See e.g., EP 2 100 949).

In addition, in some embodiments and as further described herein, the cleaning compositions of the present disclosure further comprise at least one bleach catalyst. In some embodiments, the manganese triazacyclononane and related complexes find use, as well as cobalt, copper, manganese, and iron complexes. Additional bleach catalysts find use in the present disclosure (See e.g., U.S. Pat. Nos. 4,246,612, 5,227,084, 4,810,410, WO 99/06521, and EP 2 100 949).

In some still further embodiments, the cleaning compositions provided herein contain at least one deposition aid. Suitable deposition aids include, but are not limited to, polyethylene glycol, polypropylene glycol, polycarboxylate, soil release polymers such as polytelephthalic acid, clays such as kaolinite, montmorillonite, atapulgite, illite, bentonite, halloysite, and mixtures thereof.

As indicated herein, in some embodiments, anti-redeposition agents find use in some embodiments of the present disclosure. In some embodiments, non-ionic surfactants find use. For example, in automatic dishwashing embodiments, non-ionic surfactants find use for surface modification purposes, in particular for sheeting, to avoid filming and spotting and to improve shine. These non-ionic surfactants also find use in preventing the re-deposition of soils. In some embodiments, the anti-redeposition agent is a non-ionic surfactant as known in the art (See e.g., EP 2 100 949).

In some embodiments, the cleaning compositions of the present disclosure include one or more dye transfer inhibiting agents. Suitable polymeric dye transfer inhibiting agents include, but are not limited to, polyvinylpyrrolidone polymers, polyamine N-oxide polymers, copolymers of N-vinylpyrrolidone and N-vinylimidazole, polyvinyloxazolidones and polyvinylimidazoles or mixtures thereof. In embodiments in which at least one dye transfer inhibiting agent is used, the cleaning compositions of the present disclosure comprise from about 0.0001% to about 10%, from about 0.01% to about 5%, or even from about 0.1% to about 3% by weight of the cleaning composition.

In some embodiments, silicates are included within the compositions of the present disclosure. In some such embodiments, sodium silicates (e.g., sodium disilicate, sodium metasilicate, and crystalline phyllosilicates) find use. In some embodiments, silicates are present at a level of from about 1% to about 20%. In some embodiments, silicates are present at a level of from about 5% to about 15% by weight of the composition.

In some still additional embodiments, the cleaning compositions of the present disclosure also contain dispersants. Suitable water-soluble organic materials include, but are not limited to the homo- or co-polymeric acids or their salts, in which the polycarboxylic acid comprises at least two carboxyl radicals separated from each other by not more than two carbon atoms.

In one aspect, the amount of lipolytic enzyme variant protein of the disclosure may be 0.001-30 mg per gram of detergent or 0.001-100 mg per liter of wash liquor. The lipase variants of the disclosure are particularly suited for detergents comprising of a combination of anionic and nonionic surfactant with 70-100% by weight of anionic surfactant and 0-30% by weight of nonionic, particularly 80-100% of anionic surfactant, and 0-20% nonionic surfactant. As further described, some preferred lipases of the disclosure are also suited for detergents comprising 40-70% anionic and 30-60% non-ionic surfactant.

In some embodiments, the cleaning compositions of the present disclosure contain one or more catalytic metal complexes. In some embodiments, a metal-containing bleach catalyst finds use. In some embodiments, the metal bleach catalyst comprises a catalyst system comprising a transition metal cation of defined bleach catalytic activity, (e.g., copper, iron, titanium, ruthenium, tungsten, molybdenum, or manganese cations), an auxiliary metal cation having little or no bleach catalytic activity (e.g., zinc or aluminum cations), and a sequestrate having defined stability constants for the catalytic and auxiliary metal cations, particularly ethylenediaminetetraacetic acid, ethylenediaminetetra (methylenephosphonic acid) and water-soluble salts thereof are used (See e.g., U.S. Pat. No. 4,430,243). In some embodiments, the cleaning compositions of the present disclosure are catalyzed by means of a manganese compound. Such compounds and levels of use are well known in the art (See e.g., U.S. Pat. No. 5,576,282). In additional embodiments, cobalt bleach catalysts find use in the cleaning compositions of the present disclosure. Various cobalt bleach catalysts are known in the art (See e.g., U.S. Pat. Nos. 5,597,936 and 5,595,967) and are readily prepared by known procedures.

In some additional embodiments, the cleaning compositions of the present disclosure include a transition metal complex of a macropolycyclic rigid ligand (MRL). As a practical matter, and not by way of limitation, in some embodiments, the compositions and cleaning processes provided by the present disclosure are adjusted to provide on the order of at least one part per hundred million of the active MRL species in the aqueous washing medium, and in some embodiments, provide from about 0.005 ppm to about 25 ppm, more preferably from about 0.05 ppm to about 10 ppm, and most preferably from about 0.1 ppm to about 5 ppm, of the MRL in the wash liquor.

In some embodiments, transition-metals in the instant transition-metal bleach catalyst include, but are not limited to manganese, iron and chromium. MRLs also include, but are not limited to special ultra-rigid ligands that are cross-bridged (e.g., 5,12-diethyl-1,5,8,12-tetraazabicyclo[6.6.2]hexadecane). Suitable transition metal MRLs are readily prepared by known procedures (See e.g., WO 2000/32601, and U.S. Pat. No. 6,225,464).

In some embodiments, the cleaning compositions of the present disclosure comprise metal care agents. Metal care agents find use in preventing and/or reducing the tarnishing, corrosion, and/or oxidation of metals, including aluminum, stainless steel, and non-ferrous metals (e.g., silver and copper). Suitable metal care agents include those described in EP 2 100 949, WO 9426860 and WO 94/26859). In some embodiments, the metal care agent is a zinc salt. In some further embodiments, the cleaning compositions of the present disclosure comprise from about 0.1% to about 5% by weight of one or more metal care agents.

The detergent composition may, in addition to the lipolytic enzyme variant of the disclosure, comprise other enzyme(s) providing cleaning performance and/or fabric care benefits, e.g. proteases, additional lipases, cutinases, amylases, cellulases, peroxidases, oxidases (e.g. laccases), mannanases, oxidoreductases, and/or pectate lyases.

In some embodiments, the cleaning compositions of the present disclosure comprise one or more additional detergent enzymes, which provide cleaning performance and/or fabric care and/or dishwashing benefits. Examples of suitable enzymes include, but are not limited to, hemicellulases, cellulases, perhydrolases, peroxidases, lipolytic enzymes, xylanases, lipases, phospholipases, esterases, cutinases, pectinases, pectate lyases, mannanases, keratinases, reductases, oxidases, phenoloxidases, lipoxygenases, ligninases, pullulanases, tannases, pentosanases, malanases, β-glucanases, arabinosidases, hyaluronidases, chondroitinases, laccases, and amylases, or any combinations or mixtures thereof. In some embodiments, a combination of enzymes is used (i.e., a “cocktail”) comprising conventional applicable enzymes like lipolytic enzyme, lipase, cutinase and/or cellulase in conjunction with amylase is used.

For example, a lipolytic enzyme variant of the disclosure can be combined with a protease. Suitable proteolytic enzymes include those of animal, vegetable or microbial origin. In some embodiments, microbial proteolytic enzymes are used. In some embodiments, the proteolytic enzyme is preferably an alkaline microbial proteolytic enzyme or a trypsin-like proteolytic enzyme. Examples of alkaline lipolytic enzymes include lipases, especially those derived from Bacillus (e.g., lentus, amyloliquefaciens, Carlsberg, 309, 147 and 168). Additional examples include those mutant proteolytic enzymes described in U.S. Pat. Nos. RE 34,606, 5,955,340, 5,700,676, 6,312,936, and 6,482,628, all of which are incorporated herein by reference. Additional protease examples include, but are not limited to trypsin (e.g., of porcine or bovine origin), and the Fusarium protease enzyme described in WO 89/06270. In some embodiments, commercially available protease enzymes that find use in the present disclosure include, but are not limited to MAXATASE®, MAXACAL™, MAXAPEM™, OPTICLEAN®, OPTIMASE®, PROPERASE®, PURAFECT®, PURAFECT® OXP, PURAMAX™, EXCELLASE™, and PURAFAST™ (Genencor); ALCALASE®, SAVINASE®, PRIMASE®, DURAZYM™, POLARZYME®, OVOZYME®, KANNASE®, LIQUANASE®, NEUTRASE®, RELASE® and ESPERASE® (Novozymes); BLAP™ and BLAP™ variants (Henkel Kommanditgesellschaft auf Aktien, Duesseldorf, Gennany), and KAP (B. alkalophilus lipase; Kao Corp., Tokyo, Japan). Various proteolytic enzymes are described in WO95/23221, WO 92/21760, U.S. Pat. Publ. No. 2008/0090747, and U.S. Pat. Nos. 5,801,039, 5,340,735, 5,500,364, 5,855,625, US RE 34,606, 5,955,340, 5,700,676, 6,312,936, and 6,482,628, and various other patents. In some further embodiments, metalloprotease enzymes find use in the present disclosure, including but not limited to the neutral metalloprotease enzyme described in WO 07/044993.

In one embodiment, the lipolytic enzyme variant or active fragment described herein has a protease stability that is greater that the protease stability of the parent lipolytic enzyme.

In some embodiments of the present disclosure, any suitable amylase finds use in the present disclosure. In some embodiments, any amylase (e.g., alpha and/or beta) suitable for use in alkaline solutions also find use. Suitable amylases include, but are not limited to those of bacterial or fungal origin. Chemically or genetically modified mutants are included in some embodiments. Amylases that find use in the present disclosure, include, but are not limited to α-amylases obtained from B. licheniformis (See e.g., GB 1,296,839). Commercially available amylases that find use in the present disclosure include, but are not limited to DURAMYL®, TERMAMYL®, FUNGAMYL®, STAINZYME®, STAINZYME PLUS®, STAINZYME ULTRA®, and BAN™ (Novozymes), as well as POWERASE™, RAPIDASE® and MAXAMYL® P (Genencor).

In some embodiments of the present disclosure, the cleaning compositions of the present disclosure further comprise amylases at a level from about 0.00001% to about 10% of additional amylase by weight of the composition and the balance of cleaning adjunct materials by weight of composition. In some other embodiments of the present disclosure, the cleaning compositions of the present disclosure also comprise amylases at a level of about 0.0001% to about 10%, about 0.001% to about 5%, about 0.001% to about 2%, about 0.005% to about 0.5% amylase by weight of the composition.

In some further embodiments, any suitable cellulase finds used in the cleaning compositions of the present disclosure. Suitable cellulases include, but are not limited to those of bacterial or fungal origin. Chemically or genetically modified mutants are included in some embodiments. Suitable cellulases include, but are not limited to Humicola insolens cellulases (See e.g., U.S. Pat. No. 4,435,307). Especially suitable cellulases are the cellulases having color care benefits (See e.g., EP 0 495 257). Commercially available cellulases that find use in the present include, but are not limited to CELLUZYME®, CAREZYME® (Novozymes), and KAC-500(B)™ (Kao Corporation) PURADAX HA 1200E (Danisco), PURADAX EG 7000L (Danisco). In some embodiments, cellulases are incorporated as portions or fragments of mature wild-type or variant cellulases, wherein a portion of the N-terminus is deleted (See e.g., U.S. Pat. No. 5,874,276). In some embodiments, the cleaning compositions of the present disclosure further comprise cellulases at a level from about 0.00001% to about 10% of additional cellulase by weight of the composition and the balance of cleaning adjunct materials by weight of composition. In some other embodiments of the present disclosure, the cleaning compositions of the present disclosure also comprise cellulases at a level of about 0.0001% to about 10%, about 0.001% to about 5%, about 0.001% to about 2%, about 0.005% to about 0.5% cellulase by weight of the composition.

Any mannanase suitable for use in detergent compositions also finds use in the present disclosure. Suitable mannanases include, but are not limited to those of bacterial or fungal origin. Chemically or genetically modified mutants are included in some embodiments. Various mannanases are known which find use in the present disclosure (See e.g., U.S. Pat. Nos. 6,566,114, 6,602,842, and 6,440,991, all of which are incorporated herein by reference). In some embodiments, the cleaning compositions of the present disclosure further comprise mannanases at a level from about 0.00001% to about 10% of additional mannanase by weight of the composition and the balance of cleaning adjunct materials by weight of composition. In some embodiments of the present disclosure, the cleaning compositions of the present disclosure also comprise mannanases at a level of about 0.0001% to about 10%, about 0.001% to about 5%, about 0.001% to about 2%, about 0.005% to about 0.5% mannanase by weight of the composition.

In some embodiments, peroxidases are used in combination with hydrogen peroxide or a source thereof (e.g., a percarbonate, perborate or persulfate) in the compositions of the present disclosure. In some alternative embodiments, oxidases are used in combination with oxygen. Both types of enzymes are used for “solution bleaching” (i.e., to prevent transfer of a textile dye from a dyed fabric to another fabric when the fabrics are washed together in a wash liquor), preferably together with an enhancing agent (See e.g., WO 94/12621 and WO 95/01426). Suitable peroxidases/oxidases include, but are not limited to those of plant, bacterial or fungal origin. Chemically or genetically modified mutants are included in some embodiments. In some embodiments, the cleaning compositions of the present disclosure further comprise peroxidase and/or oxidase enzymes at a level from about 0.00001% to about 10% of additional peroxidase and/or oxidase by weight of the composition and the balance of cleaning adjunct materials by weight of composition. In some other embodiments of the present disclosure, the cleaning compositions of the present disclosure also comprise, peroxidase and/or oxidase enzymes at a level of about 0.0001% to about 10%, about 0.001% to about 5%, about 0.001% to about 2%, about 0.005% to about 0.5% peroxidase and/or oxidase enzymes by weight of the composition.

In some embodiments, additional enzymes find use, including but not limited to perhydrolases (See e.g., WO 05/056782). In addition, in some embodiments, mixtures of the above-mentioned enzymes are encompassed herein, in particular one or more additional lipolytic enzymes, amylase, protease, mannanase, and/or at least one cellulase. Indeed, it is contemplated that various mixtures of these enzymes will find use in the present disclosure. It is also contemplated that the varying levels of the lipolytic enzyme variant(s) and one or more additional enzymes may both independently range to about 10%, the balance of the cleaning composition being cleaning adjunct materials. The specific selection of cleaning adjunct materials is readily made by considering the surface, item, or fabric to be cleaned, and the desired form of the composition for the cleaning conditions during use (e.g., through the wash detergent use).

The enzymes of the detergent composition may be stabilized using conventional stabilizing agents (e.g. a polyol such as propylene glycol or glycerol, a sugar or sugar alcohol, lactic acid, boric acid, or a boric acid derivative as e.g. an aromatic borate ester). Boronic acid or borinic acid derivatives as enzyme stabilizers include Boric acid, Thiophene-3-boronic acid, Thiophene-2-boronic acid, 4-Methylthiophene-2-boronic acid, 5-Ethylthiophene-2-boronic acid, 5-Methylthiophene-2-boronic acid, 5-Bromothiophene-2-boronic acid, 5-Chlorothiophene-2-boronic acid, Dibenzothiophene-1-boronic acid, Dibenzofuran-1-boronic acid, Dibenzofuran-4-boronic acid, Picoline-2-boronic acid, Diphenylborinic acid (ethanolamine complex), 5-Methoxythio-phene-2-boronic acid, Thionaphthrene-1-boronic acid, Furan-2-boronic acid, Furan-3-boronic acid, 2,5-dimethyl-thiophene-3-boronic acid, Benzofuran-1-boronic acid, 3-Methoxythio-phene-2-boronic acid, 5-n-Propyl-thiophene-2-boronic acid, 5-Methoxyfuran-2-boronic acid, 3-Bromothiophene-2-boronic acid, 5-Ethylfuran-2-boronic acid, 4-Carbazole ethyl boronic acid.

In some embodiments, the enzymes used in the cleaning compositions are stabilized by any suitable technique. In some embodiments, the enzymes employed herein are stabilized by the presence of water-soluble sources of calcium and/or magnesium ions in the finished compositions that provide such ions to the enzymes. In some embodiments, the enzyme stabilizers include oligosaccharides, polysaccharides, and inorganic divalent metal salts, including alkaline earth metals, such as calcium salts. It is contemplated that various techniques for enzyme stabilization will find use in the present disclosure. For example, in some embodiments, the enzymes employed herein are stabilized by the presence of water-soluble sources of zinc (II), calcium (II) and/or magnesium (II) ions in the finished compositions that provide such ions to the enzymes, as well as other metal ions (e.g., barium (II), scandium (II), iron (II), manganese (II), aluminum (III), Tin (II), cobalt (II), copper (II), nickel (II), and oxovanadium (IV). Chlorides and sulfates also find use in some embodiments of the present disclosure. Examples of suitable oligosaccharides and polysaccharides (e.g., dextrins) are known in the art (See e.g., WO 07/145964). In some embodiments, reversible enzyme inhibitors also find use, such as boron-containing compounds (e.g., borate, 4-formyl phenyl boronic acid) and/or a tripeptide aldehyde find use to further improve stability, as desired.

An optional ingredient in cleaning compositions of the present disclosure is a suds suppressor (e.g. exemplified by silicones-alkylated polysiloxane materials, and silica-silicone mixtures, where the silica is in the form of silica aerogels and xerogels and hydrophobic silicas of various types. The suds suppressor can be incorporated as particulates, in which the suds suppressor is advantageously releasable incorporated in a water-soluble or water dispersible, substantially non surface-active detergent impermeable carrier. Alternatively, the suds suppressor can be dissolved or dispersed in a liquid carrier and applied by spraying on to one or more of the other components.

The detergent may also contain inorganic or organic softening agents. Inorganic softening agents are exemplified by the smectite clays (5% to 15%). Organic fabric softening agents (0.5% to 5%) include the water insoluble tertiary amines and their combination with mono C₁₂-C₁₄ quaternary ammonium salts and di-long-chain amides, or high molecular weight polyethylene oxide materials.

The detergent may also contain other conventional detergent ingredients such as, e.g., fabric conditioners including clays, deflocculant material, foam boosters/foam depressors (in dishwashing detergents foam depressors), anti-corrosion agents, soil-suspending or dispersing agents (0 to 10%), anti-soil-redeposition agents, dyes, dehydrating agents, bactericides, optical brighteners, abrasives, tarnish inhibitors, coloring agents, and/or encapsulated or non-encapsulated perfumes.

Some examples of detergent formulations are shown in Tables 1-11.

TABLE 1
Liquid detergent formulation

| | |
|---|---|
| Nonionic (Neodol 25-7) AE | 25% |
| Anionic (Vista C-S50) LAS | 5% |
| Triethanolamine | 5% |
| Ethanol | 10% |
| Stabilizer | 0.5, 2.5, 5% |
| Protease | 1% |
| Amylase | 0.3% |
| Water | up to 100% |
| Adjust to pH = 9.0 | |
| Lipase insert after amylase | 0.001-1% |

TABLE 2
Detergent formulations I, II, II, IV, V and VI.
Detergent formulations

| | I (%) | II (%) | III (%) | IV (%) | V (%) | VI (%) |
|---|---|---|---|---|---|---|
| Ingredients | powder | powder | powder | powder | liquid | liquid |
| Linear alkylbenzenesulfonate | 7-12 | 6-11 | 5-9 | 8-12 | 15-21 | 15-21 |
| (calculated as acid) or slkyl | | | | | | |
| sulfate, alpha olefin | | | | | | |
| sulfonate, alpha-sulfo fatty | | | | | | |
| acid methyl esters, | | | | | | |
| alkanesulfonates, soap | | | | | | |
| Alcohol ethoxysulfate (e.g. | 1-4 | 1-3 | — | | | |
| C₁₂₋₁₈ alcohol 1-2 EO) or | | | | | | |
| alkyl sulfate (e.g. C₁₆₋₁₈) | | | | | | |
| soap as fatty acid | — | — | 1-3 | | 3-13 | 3-10 |
| (e.g. C₁₆₋₂₂ or oleic acid) | | | | | | |
| Alcohol ethoxylate (e.g. | 5-9 | 5-9 | 7-14 | 10-25 | 12-18 | 3-9 |
| C₁₄₋₁₅ or C₁₂₋₁₅ 7EO or 5EO) | | | | | | |
| Alkenylsuccinic acid (C₁₂₋₁₄) | | | | | 0-13 | |
| Aminoethanol | | | | | 8-18 | |
| sodium carbonate (Na₂CO₃) | 14-20 | 15-21 | 10-17 | 14-22 | | |
| soluble silicate (as Na₂O, | 2-6 | 1-4 | 3-9 | 1-5 | | |
| 2SiO₂) | | | | | | |
| zeolite (as NaAlSiO₄) | 15-22 | 24-34 | 23-33 | 25-35 | | 14-22 |
| sodium sulfate (as Na₂SO₄) | 0-6 | 4-10 | 0-4 | 0-10 | | |
| sodium citrate/citric acid | 0-15 | 0-15 | — | | 2-8 | 9-18 |
| (C₆H₅Na₃O₇/C₆H₈O₇) or | | | | | | |
| potassium citrate | | | | | | |
| sodium perborate (as | 11-18 | — | 8-16 | | 0-2 | 0-2 |
| NaBO₃•H₂O) or borate | | | | | | |
| (as B₄O₇) | | | | | | |
| TAED | 2-6 | — | 2-8 | | | |
| Phosphonate (e.g. EDTMPA) | — | — | 0-1 | | 0-3 | |
| Ethanol | | | | | 0-3 | |
| carboxymethy1cellulose | 0-2 | 0-2 | 0-2 | 0-2 | | 0-2 |
| Polymers (PEG, PVP) | | | | | 0-3 | 0-3 |
| Anchoring polymers (e.g. | 0-3 | 1-6 | 1-3 | 1-3 | | 0-3 |
| maleic/acrylic acid | | | | | | |
| copolymer PVP, PEG) | | | | | | |
| Propylene glycol | | | | | 8-14 | |
| Glycerol | | | | | | 0-5 |
| Enzymes (alkaline lipases) | 0-5 | 0-5 | 0-5 | 0-5 | 0-5 | 0-5 |
| minor ingredients (e.g. | 0-5 | 0-5 | 0-5 | 0-5 | 0-5 | 0-5 |
| suds, supressors, perfume, | | | | | | |
| optical brightener, | | | | | | |
| photobleach) | | | | | | |

TABLE 3
Detergent formulations VII, VIII, IX, X, XI and XII
Detergent formulations

| | VII (%) | VIII (%) | IX (%) | X (%) | XI (%) | XII (%) |
|---|---|---|---|---|---|---|
| Ingredients | powder | powder | powder | liquid | liquid | powder |
| Linear alkylbenzenesulfonate | | 8-14 | 6-12 | 15-23 | 20-32 | 25-40 |
| (calculated as acid) or slkyl | | | | | | |
| sulfate, alpha olefin | | | | | | |
| sulfonate, alpha-sulfo fatty | | | | | | |
| acid methyl esters, | | | | | | |
| alkanesulfonates, soap | | | | | | |
| Fatty alcohol sulfate | 5-10 | | | | | |
| Ethoxylated fatty acid | 3-9 | 5-11 | | | | |
| monoethanolamide | | | | | | |
| Alcohol ethoxysulfate (e.g. | | | | 8-15 | | |
| C₁₂₋₁₈ alcohol 1-2 EO, or | | | | | | |
| C₁₂₋₁₅ 2-3 EO) or alkyl | | | | | | |
| sulfate (e.g. C₁₆₋₁₈) | | | | | | |
| soap as fatty acid | 0-3 | 0-3 | 2-6 | 0-3 | | |
| (e.g. C₁₆₋₂₂ or oleic | | | | | | |
| acid or lauric acid) | | | | | | |
| Alcohol ethoxylate (e.g. | | | 1-4 | 3-9 | 6-12 | 1-10 |
| C₁₄₋₁₅ or C₁₂₋₁₅ 7EO or 5EO) | | | | | | |
| Alkenylsuccinic acid (C₁₂₋₁₄) | | | | | | |
| Aminoethanol | | | | 1-5 | 2-6 | |
| sodium carbonate (Na₂CO₃) | 5-10 | 4-10 | 14-22 | | | 8-25 |
| soluble silicate (as Na₂O, | 1-4 | 1-4 | | | | 5-15 |
| 2SiO₂) | | | | | | |
| zeolite (as NaAlSiO₄) | 20-40 | 30-50 | 18-32 | | | 15-28 |
| sodium sulfate (as Na₂SO₄) | 2-8 | 3-11 | 5-20 | | | 0-5 |
| sodium citrate/citric acid | | 5-12 | 3-8 | 5-10 | 8-14 | |
| (C₆H₅Na₃O₇/C₆H₈O₇) or | | | | | | |
| potassium citrate | | | | | | |
| Hydrotrope (eg sodium | | | | 2-6 | | |
| toluenesulfonate) | | | | | | |
| sodium perborate (as | 12-18 | | 4-9 | 0-2 | 1-3 | 0-20 |
| NaBO₃•H₂O, or NaBO₃•4H₂O) | | | | | | |
| or borate (as B₄O₇) | | | | | | |
| TAED (or NOBS) | 2-7 | | 1-5 | | | 0-5 |
| Phosphonate (e.g. EDTMPA) | | | | | | |
| Ethanol | | | | 1-3 | | |
| carboxymethy1cellulose | | | 0-2 | 0-1 | | |
| Polymers (PEG, PVP) | | | | | | |
| Anchoring polymers (e.g. | 1-5 | 1-5 | 1-5 | | 0-3 | |
| maleic/acrylic acid | | | | | | |
| copolymer PVP, PEG) | | | | | | |
| Propylene glycol | | | | 2-5 | | |
| Glycerol | | | | | 3-8 | |
| Enzymes (alkaline lipases) | 0-5 | 0-5 | 0-5 | 0-5 | 0-5 | 0-5 |
| minor ingredients (e.g. suds, | 0-5 | 0-5 | 0-5 | 0-5 | 0-5 | 0-3 |
| supressors, perfume, optical | | | | | | |
| brightener, photobleach) | | | | | | |

TABLE 4
Detergent composition Ex1, Ex2, Ex3 and Ex4
Detergent composition

| | Ex. 1 | Ex. 2 | Ex. 3 | Ex. 4 |
|---|---|---|---|---|
| | Level | Level | Level | Level |
| Ingredients | (parts | (parts | (parts | (parts |
| Material | as is) | as is) | as is) | as is) |
| Glycerol | 3.17 | 3.17 | 3.17 | 3.17 |
| MPG | 5.7 | 5.7 | 5.7 | 5.7 |
| NaOH | 2.13 | 2.13 | 2.13 | 2.13 |
| TEA | 2.05 | 2.05 | 2.05 | 2.05 |
| Neodol 25-7 | 12.74 | 12.74 | 12.74 | 12.74 |
| F-Dye | 0.18 | 0.18 | 0.18 | 0.18 |
| Citric Acid | 1.71 | 1.71 | 1.71 | 1.71 |
| LAS (as LAS Acid) | 8.49 | 8.49 | 8.49 | 8.49 |
| Fatty acid | 3.03 | 3.03 | 3.03 | 3.03 |
| Empigen BB | 1.5 | 1.5 | 1.5 | 1.5 |
| SLES | 4.24 | 4.24 | 4.24 | 4.24 |
| Dequest 2066 | 0.875 | 0.875 | 0.875 | 0.875 |
| Patent Blue | 0.00036 | 0.00036 | 0.00036 | 0.00036 |
| Acid Yellow | 0.00005 | 0.00005 | 0.00005 | 0.00005 |
| Opacifier | 0.0512 | 0.0512 | 0.0512 | 0.0512 |
| Perfume | 0.734 | 0.734 | 0.734 | 0.734 |
| Borax | 10 | 10 | 10 | 10 |
| Savinase | 2.362 | 2.362 | 2.362 | 2.362 |
| Stainzyme | 0.945 | 0.945 | 0.945 | 0.945 |
| Soap | 3.03 | 3.03 | 3.03 | 3.03 |
| EPEI 20E0 (ex Nippon | 5.5 | 5.5 | 5.5 | 9 |
| Shokubai) | | | | |
| polyethyleneimine | | | | |
| having a weight | | | | |
| average molecular | | | | |
| weight of about 600, | | | | |
| and wherein the | | | | |
| polyethyleneimine | | | | |
| has been modified | | | | |
| by alkoxylation with | | | | |
| an average 20 ethylene | | | | |
| oxide moieties | | | | |
| LIPASE | 3 | 3 | 3 | 3 |
| Texcare SRN170 | 0 | 7.5 | 0 | 0 |
| (ex Clariant) soil | | | | |
| release polymer | | | | |
| Sokolan CP5 (ex BASF) | 0 | 0 | 20 | 0 |
| Soil-release polymer | | | | |

TABLE 5
Enzymatic detergent and bleaching composition A
Enzymatic detergent and bleaching composition

| Ingredients | % by weight |
|---|---|
| Sodium dodecyl benzene sulphonate | 6.5 |
| C14-C15 primary alcohol, condensed with 11 | 2 |
| moles of ethylene oxide | |
| Sodium stearate | 1 |
| Sodium silicate | 7 |
| Sodium carboxymethyl cellulose | 0.5 |
| Na₂SO₄ | 37 |
| Pentasodium triphosphate | 15 |
| Trisodium orthophosphate | 5 |
| Fluorescer | 0.2 |
| Ethylene diamine tetraacetic acid | 0.5 |
| Water | 6.2 |
| Dyes | 0.01 |
| Lipase | 0.001-1 |
| bleach systems | sodium perborate + |
| | SNOBS, sodium |
| | perborate + TAED, |
| | DPDA, MPS All |
| | generating 1.5 mmol |
| | peracid in solution |

TABLE 6
Enzymatic detergent and bleaching composition B
Enzymatic detergent and bleaching composition

| Ingredients | % by weight |
|---|---|
| Sodium dodecyl benzene sulphonate | 8.5 |
| C14-C15 primary alcohol, condensed with 11 | 4 |
| moles of ethylene oxide | |
| sodium hardened rapeseed oil soap | 1.5 |
| sodium triphosphate | 33 |
| sodium carbonate | 5 |
| sodium silicate | 6 |
| sodium sulphate | 20 |
| water | 9 |
| fluorescers, soil-suspending | minor |
| agents, dyes, perfumes | amount |
| anti-foam granules | 1.2 |
| Dequest R 2047 (34% pure) | 0.3 |
| Lipase | 0.001-1 |

TABLE 7
Enzymatic Detergent compositions
Detergent compositions

| Ingredients | % wt | % wt |
|---|---|---|
| sodium alkylbenzenesulphonate | 24 | 28 |
| pentasodium tripolyphosphate | 15 | 2.1 |
| alkaline sodium silicate sodium | 10 | 12 |
| carboxymethylcellulose sodium | 0.6 | 0.6 |
| sulphate | 32.5 | 15.4 |
| fluorescer | 0.4 | 0.4 |
| sodium carbonate | 10 | 35 |
| miscellaneous + water | to 100% | to 100% |
| Lipase | 0.001-1 | 0.001-1 |

TABLE 8
Enzymatic Detergent compositions
Enzymatic Detergent composition

| Ingredients | % by weight |
|---|---|
| sodium linear dodecylbenzenesulphonate | 13.35 |
| sodium C₁₂-C₁₃ alcohol (6.5 E0) sulphate | 6.67 |
| sodium carbonate | 54.2 |
| sodium tripolyphosphate | 9.01. |
| sodium silicate | 4.6 |
| sodium hydroxide | 1.66 |
| sodium carboxymethylcellulose | 0.5 |
| Dequest 2006 | 1.9 |
| perfume, dye, water | q.s. |
| Lipase | 0.001-1 |
| Protease (Alcalase) | 20 GU/mL |

TABLE 9
Liquid laundry detergent formulation
Liquid laundry detergent formulation

| Ingredients | Parts by weight |
|---|---|
| Sodium dodecyl benzene sulphonate | 8.5 |
| C12-C15 primary alcohol, condensed | 4 |
| with 7 moles of ethylene oxide | |
| Sodium-hardened rapeseed oil soap | 1.5 |
| Sodium triphosphate | 33 |
| Sodium carbonate | 5 |
| Sodium silicate | 6 |
| Sodium sulphate | 20 |
| Water | 9 |
| Fluorescers, soil-suspending agents, dyes, perfumes | minor amount |
| Sodium perborate | 12 |
| Tetraacetyl ethylene diamine (TAED) (granules) | 2 |
| Proteolytic enzyme (Savinase ex NOVO) | 0.4 |
| Lipase | 0.001-1 |
| Protease (Alcalase) | 20 GU/mL |

TABLE 10
Liquid detergent compositions A, B, C and D
Liquid detergent compositions

| | A | B | C | D |
|---|---|---|---|---|
| sodium dodecylbenzene sulphonate | 9 | 9 | 9 | 9 |
| C13-C15 linear primary alcohol, | 1 | 4 | 4 | 1 |
| condensed with 7 moles of ethylene | | | | |
| oxide (e.g. Synperonic A7) | | | | |
| C13-C15 linear primary alcohol, | 3 | 0 | 0 | 3 |
| condensed with 3 moles of ethylene | | | | |
| oxide (e.g. Synperonic A3) | | | | |
| sodium tripolyphosphate | 23 | 23 | 0 | 0 |
| zeolite type 4A | 0 | 0 | 24 | 24 |
| copolymer of acrylic acid with maleic | 0 | 0 | 4 | 4 |
| anhydride | | | | |
| sodium polyacrylate | 2 | 2 | 0 | 0 |
| alkaline silicate | 5 | 5 | | |
| fluorescer | 0.25 | 0.25 | 0.16 | 0.16 |
| EDTA | 0.15 | 0.15 | 0.18 | 0.18 |
| SCMC | 0.5 | 0.5 | 0.55 | 0.55 |
| salt | 2 | 2 | 0 | 0 |
| sodium sulphate | 26.8 | 26.8 | 22.31 | 22.31 |
| sodium carbonate | 0 | 0 | 10.3 | 10.3 |
| moisture | 10 | 10 | 11 | 11 |
| TAED | 3 | 3 | 3.3 | 3.3 |
| sodium perborate monohydrate | 10 | 10 | 8 | 8 |
| calcium Dequest ®²⁰⁴⁷ | 0.7 | 0.7 | 0.3 | 0.3 |
| foam depressor | 3 | 3 | 2.5 | 2.5 |
| perfume | 0.2 | 0.2 | 0 | 0 |
| alkaline protease (Savinase (A) 6T) | 0.4 | 0.4 | 0.4 | 0.4 |

| | |
|---|---|
| Lipase | 0.001-1 |

TABLE 11
Dishwashing composition
Dishwashing composition

| Ingredients | % by weight |
|---|---|
| sodium tripolyphosphate | 24 |
| soda ash | 20 |
| sodium disilicate | 11 |
| linear C10 alcohol, condensed with 6 moles of | 2.5 |
| ethylene oxide and 24 moles of propylene oxide | |
| sodium sulphate | 44 |
| water | to 100 |
| Lipase | 0.001-1 |

In one aspect the deterent is an Anionic Model Detergent A, comprising a model granular detergent (90% anionic out of total surfactants, pH in solution 10.2) made by mixing the following ingredients (% by weight):8.7% anionic surfactant: LAS (C₁₀-C₁₃), 7.4% anionic surfactant: AS (C₁₂), 1.8% Nonionic surfactant: alcohol ethoxylate (C₁₂-C₁₅ 7EO), 30% Zeolite P (Wessalite P), 18% Sodium Carbonate, 5% Sodium Citrate, 17% Sodium sulfate, 0.3% Carboxy-Methyl-Cellulose, 6.5% Sodium-percarbonate monohydrate, and 2.1% NOBS.

In one aspect the deterent is an Anionic Model Detergent B, comprising a second model granular detergent (79% anionic out of total surfactants, pH in solution 10.2) made by mixing the following ingredients (% by weight): 27% anionic surfactant: AS (C₁₂), 7% Nonionic surfactant (C₁₂₋₁₅, 7EO), 60% Zeolite P (Wessalite P), 5% Sodium Carbonate, 0.6% Sokalan CPS, 1.5% Carboxy-Methyl-Cellulose.

In one aspect the deterent is an Anionic/non-ionic Model Detergent C, comprising a model detergent solution (32% anionic out of total surfactant, pH 10.2) that is made by adding the following ingredients to 3.2 mM Ca2+/Mg2+(5:1) in pure water: 0.300 g/l of alkyl sulphate (AS; C₁₄₋₁₆); 0.650 g/l of alcohol ethoxylate (AEO; C₁₂₋₁₄, 6EO); 1.750 g/l of Zeolite P, 0.145 g/l of Na₂CO₃, 0.020 g/l of Sokalan CPS and 0.050 g/l of CMC (carboxy-methyl cellulose)

In one aspect the detergent is a European laundry powder detergent comprising the following ingredients: 15% of surfactant of which 6% was LAS, 3% was AES and 6% was non ionic surfactants, and further contained 47% builder comprising fatty acid, zeolite A, carbonate and silicate; or 15% of surfactant of which 3% was AES, 6% was LAS and 6% was non ionic surfactants, and further comprised 47% builder comprising fatty acid, zeolite A, carbonate, silicate, and it comprised 5% polycarboxylate polymers; or 15% of surfactant of which 3% was AES, 6% was LAS and 6% was non ionic surfactants, and further contained 47% builder comprising fatty acid, zeolite A, carbonate, silicate, and it comprised 5% polycarboxylate polymers; or 15% of surfactant of which 6% was LAS, 3% was AES and 6% was nonionic surfactants and further contained 47% builder consisting of fatty acid, zeolite A, carbonate & silicate, 5% polycarboxylate dispersing polymers, 15% sodium perborate, and 4% tetraacetyl-ethylene-diamine (TAEO); or 15% of surfactant of which 6% was LAS, 3% was AES and 6% was non ionic surfactants and further contained 47% builder consisting of fatty acid, 22% zeolite A, carbonate and silicate, and 5% polycarboxylate dispersing polymers; or 15% of surfactant of which 6% was LAS, 3% was AES and 6% was non ionic surfactants and further contained 47% builder consisting of fatty acid, 22% zeolite A, carbonate and silicate, and 5% polycarboxylate dispersing polymers; or 15% of surfactant of which 6% was LAS, 3% was AES and 6% was nonionic surfactants and further contained 47% builder consisting of fatty acid, 22% zeolite A, carbonate and silicate, and 5% polycarboxylate dispersing polymers; or 21% of surfactant of which 8.1% was LAS, 6.5% was AS, 4.0% was non ionic surfactants, and 2.5% was cationic surfactants (DSDMAC) and further contained 64% builder consisting of fatty acid, carbonate, zeolite A, silicates, and citrate, and also contained 2.7% of dispersing polymers; or 16.9% surfactants including soap of which 11% was LAS and 5.9% non-ionic and 4.1% soap, and 63% builders.

In one aspect the detergent is a European Liquid Laundry Detergent comprising the following ingredients: 27% of surfactant of which 16.9% was AS, 6.7% was nonionic surfactants, and 3.5% was cationic surfactants (DSDMAC) and further contained 18.7% builder consisting of fatty acid, carbonate, citrate, and boric acid.

In one aspect the detergent is a North American Laundry Liquid detergent comprising the following ingredients: 23% of surfactant of which 16% was AES, 5% was LAS and 2% was non ionic surfactants. It further contained 6% builder comprising soap, citric acid, DTPA and calcium formate; or 23% of surfactant of which 16% was AES, 5% was LAS and 2% was non ionic surfactants. It further contained 6% builder consisting of soap, citric acid, DTPA and calcium formate, and 5% poly-carboxylate dispersing polymers.

In one aspect the detergent is a North American Laundry Powder detergent comprising the following ingredients: 16.3% of surfactant of which 7.8% was LAS, 6.7% was AS and 1.8% was nonionic surfactants, and 60% builder comprising fatty acid, zeolite A, carbonate and silicate.; or 4.9% of surfactant of which 11.5% was LAS and 3.4% was non ionic surfactants, and 55% builder comprising fatty acid, zeolite A, carbonate and silicate; or 19.5% of surfactant of which 4.5% was LAS, 13% was AS and 2% was non ionic surfactants, and 61% builder comprising fatty acid, zeolite A, carbonate and silicate.

In one aspect the detergent is a Japanese laundry powder detergent comprising the following ingredients: 24.3% of surfactant of which 11.1% was LAS, 11.6% was ester sulfonate and 1.6% was nonionic surfactants, and 60% builder comprising fatty acid, zeolite A, carbonate and silicate; or 27.9% of surfactant of which 15 27.5% was LAS and 0.4% was nonionic surfactants, and 64% builder comprising zeolite A, carbonate, citrate, phosphates and silicate.

In one aspect the detergent is a European color compact laundry powder comprising the following ingredients: 21.1% of a surfactant system, of which 8.1% was LAS, 6.5% was AS, 2.5% was Arguat 2T-70, and 4% was non-ionic surfactants, and 64% builder comprising fatty acid, zeolite A, carbonate, citric acid and silicate. The surfactant system was prepared separately from the builder. The surfactant system was prepared either Neodol25-7 or Lutensol ON60 as nonionic surfactant.

The cleaning compositions of the present disclosure are advantageously employed for example, in laundry applications, hard surface cleaning and dishwashing applications.

In some aspects, due to the unique advantage of increased performance at low temperature, the lipolytic enzyme variants described herein are ideally suited for laundry applications. Furthermore, the lipolytic enzyme variants of the present disclosure find use in granular and liquid compositions.

The lipolytic enzyme variants of the present disclosure also find use in cleaning additive products. Such additive products are intended to supplement and/or boost the performance of conventional detergent compositions and can be added at any stage of the cleaning process.

In some embodiments, the present disclosure provides cleaning additive products including at least one enzyme of the present disclosure that is suited for inclusion in a wash process when additional bleaching effectiveness is desired. In some embodiments, the additive product is in its simplest form, one or more lipolytic enzymes. In some embodiments, the additive is packaged in dosage form for addition to a cleaning process. In some embodiments, the additive is packaged in dosage form for addition to a cleaning process where a source of peroxygen is employed and increased bleaching effectiveness is desired. Any suitable single dosage unit form finds use with the present disclosure, including but not limited to pills, tablets, gelcaps, or other single dosage units such as pre-measured powders or liquids. In some embodiments, filler(s) or carrier material(s) are included to increase the volume of such compositions. Suitable filler or carrier materials include, but are not limited to, various salts of sulfate, carbonate and silicate as well as talc, clay and the like. Suitable filler or carrier materials for liquid compositions include, but are not limited to water or low molecular weight primary and secondary alcohols including polyols and diols. Examples of such alcohols include, but are not limited to, methanol, ethanol, propanol and isopropanol. In some embodiments, the compositions contain from about 5% to about 90% of such materials. Acidic fillers find use to reduce pH. Alternatively, in some embodiments, the cleaning additive includes adjunct ingredients, as more fully described below.

The present cleaning compositions and cleaning additives can comprise at least one of the lipolytic enzyme variants provided herein, alone or in combination with other lipolytic enzymes and/or additional enzymes.

In some aspects, the cleaning compositions herein are formulated such that, during use in aqueous cleaning operations, the wash water will have a pH of from about 5.0 to about 11.5, or about 6.0 to 8.0 or even from about 7.5 to about 10.5. Liquid product formulations are typically formulated to have a neat pH from about 3.0 to about 9.0 or even from about 3 to about 8. Granular laundry products are typically formulated to have a pH from about 6 to about 11, or even from about 8 to about 10. Techniques for controlling pH at recommended usage levels include the use of buffers, alkalis, acids, etc., and are well known to those skilled in the art.

Suitable “low pH cleaning compositions” typically have a neat pH of from about 3 to about 8, and are typically free of surfactants that hydrolyze in such a pH environment. Such surfactants include sodium alkyl sulfate surfactants that comprise at least one ethylene oxide moiety or even from about 1 to about 16 moles of ethylene oxide. Such cleaning compositions typically comprise a sufficient amount of a pH modifier, such as sodium hydroxide, monoethanolamine or hydrochloric acid, to provide such cleaning composition with a neat pH of from about 3 to about 8. Such compositions typically comprise at least one acid stable enzyme. In some embodiments, the compositions are liquids, while in other embodiments, they are solids. The pH of such liquid compositions is typically measured as a neat pH. The pH of such solid compositions is measured as a 10% solids solution of said composition wherein the solvent is distilled water. In these embodiments, all pH measurements are taken at 20° C., unless otherwise indicated.

In some embodiments, when the lipolytic enzyme variant(s) is/are employed in a granular composition or liquid, it is desirable for the lipolytic enzyme variant to be in the form of an encapsulated particle to protect the lipolytic enzyme variant from other components of the granular composition during storage. In addition, encapsulation is also a means of controlling the availability of the lipolytic enzyme variant during the cleaning process. In some embodiments, encapsulation enhances the performance of the lipolytic enzyme variant(s) and/or additional enzymes. In this regard, the lipolytic enzyme variants of the present disclosure are encapsulated with any suitable encapsulating material known in the art. In some embodiments, the encapsulating material typically encapsulates at least part of the catalyst for the lipolytic enzyme variant(s) of the present disclosure. Typically, the encapsulating material is water-soluble and/or water-dispersible. In some embodiments, the encapsulating material has a glass transition temperature (Tg) of 0° C. or higher. Glass transition temperature is described in more detail in WO 97/11151. The encapsulating material is typically selected from consisting of carbohydrates, natural or synthetic gums, chitin, chitosan, cellulose and cellulose derivatives, silicates, phosphates, borates, polyvinyl alcohol, polyethylene glycol, paraffin waxes, and combinations thereof. When the encapsulating material is a carbohydrate, it is typically selected from monosaccharides, oligosaccharides, polysaccharides, and combinations thereof. In some typical embodiments, the encapsulating material is a starch (See e.g., EP 0 922 499; U.S. Pat. Nos. 4,977,252; 5,354,559, and 5,935,826). In some embodiments, the encapsulating material is a microsphere made from plastic such as thermoplastics, acrylonitrile, methacrylonitrile, polyacrylonitrile, polymethacrylonitrile and mixtures thereof, commercially available microspheres that find use include, but are not limited to those supplied by EXPANCEL® (Stockviksverken, Sweden), and PM 6545, PM 6550, PM 7220, PM 7228, EXTENDOSPHERES®, LUXSIL®, Q-CEL®, and SPHERICEL® (PQ Corp., Valley Forge, PA).

As described herein, the lipolytic enzyme variants of the present disclosure find particular use in the cleaning industry, including, but not limited to laundry and dish detergents. These applications place enzymes under various environmental stresses. The lipolytic enzyme variants of the present disclosure provide advantages over many currently used enzymes, due to their stability under various conditions.

Indeed, there are a variety of wash conditions including varying detergent formulations, wash water volumes, wash water temperatures, and lengths of wash time, to which lipolytic enzymes involved in washing are exposed. In addition, detergent formulations used in different geographical areas have different concentrations of their relevant components present in the wash water. For example, European detergents typically have about 2000-10000 ppm of detergent components in the wash water, while Asian detergents typically have approximately 300-2500 ppm of detergent components in the wash water. In North America, particularly the United States, detergents typically have about 300 ppm-1500 ppm of detergent components present in the wash water.

A high detergent concentration system includes detergents where greater than about 2000 ppm of the detergent components are present in the wash water. European detergents are generally considered to be high detergent concentration systems as they have approximately 2000-10000 ppm of detergent components in the wash water.

Latin American detergents are generally high suds phosphate builder detergents and the range of detergents used in Latin America can fall in both the medium and high detergent concentrations as they range from 1500 ppm to 6000 ppm of detergent components in the wash water. As mentioned above, Brazil typically has approximately 1500 ppm of detergent components present in the wash water. However, other high suds phosphate builder detergent geographies, not limited to other Latin American countries, may have high detergent concentration systems up to about 6000 ppm of detergent components present in the wash water.

Model detergent compostions useful for testing the lipolytic enzyme variant performance, such as but not limiting to, testing the thermostability of the lipolytic enzyme variant compared to a parent lipolytic enzyme include a model HDL composition comprising the following ingredients: 3.1% lauryl alcohol ethoxylate (6EO), 9% sodium lauryl ether sulphate, 3% sodium citrate, 0.8% sorbitol, 0.8% glycerol, 0.5% triethanolamine, 1% ethanol (absolute); 7.6% linear alkylbenzenesulfonate (LAS), 3.0% potassium cocoate, 2.5% propylene glycol, 0.01% 2-methyl-4-isothiazolin-3-one, 5.8% 4M NaOH and 62.9% demineralized water.

For North American (NA) and Western European (WE) heavy duty liquid laundry (HDL) detergents, heat inactivation of the enzymes present in commercially-available detergents can be performed by placing pre-weighed liquid detergent (in a glass bottle) in a water bath at 95° C. for 2 hours or at 80° C. for 8 hours. The incubation time for heat inactivation of NA and WE auto dish washing (ADW) detergents is 8 hours. Both un-heated and heated detergents are assayed within 5 minutes of dissolving the detergent to accurately determine percentage deactivated. For testing of enzyme activity in heat-inactivated detergents, working solutions of detergents are made from the heat inactivated stocks.

Appropriate amounts of water hardness (e.g., 6 gpg or 12 gpg or 17 gpg or 21 gpg) and buffer are added to the detergent solutions to match the desired conditions. The solutions are mixed by vortexing or inverting the bottles. Tables 12-13 provides information regarding some of the commercially-available detergents and test conditions that can be used. In some experiments, additional and/or other commercially available detergents find use in the following Examples.

TABLE 12
Example of Laundry and Dish Washing Conditions
for North America, Japan and Western Europe.
Laundry and Dish Washing Conditions

| | | | | | | | |
|---|---|---|---|---|---|---|---|
| | | | | | | | T |
| Region | Form | Dose | Detergent | Buffer | Gpg | pH | (° C.) |

Laundry (Heavy Duty Liquid and Granular)

| | | | | | | | | |
|---|---|---|---|---|---|---|---|---|
| NA | HDL | 0.78 | g/l | P&G TIDE ® 2X | 5 mM HEPES | 6 | 8.0 | 20 |
| WE | HDL | 5.0 | g/L | Henkel | 5 mM HEPES | 12 | 8.2 | 40 |
| | | | | PERSIL ™ | | | | |
| WE | HDG | 8.0 | g/L | P&G ARIEL ® | 2 mM Na₂ CO₃ | 12 | 10.5 | 40 |
| JPN | HDG | 0.7 | g/L | P&G TIDE ® | 2 mM Na₂ CO₃ | 6 | 10.0 | 20 |
| NA | HDG | 1.0 | g/L | P&G TIDE ® | 2 mM Na₂ CO₃ | 6 | 10.0 | 20 |

Automatic Dish Washing

| | | | | | | | | |
|---|---|---|---|---|---|---|---|---|
| WE | ADW | 3.0 | g/L | RB | 2 mM Na₂ CO₃ | 21 | 10.0 | 40 |
| | | | | CALGONIT ™ | | | | |
| NA | ADW | 3.0 | g/L | P&G | 2 mM Na₂ CO₃ | 9 | 10.0 | 40 |
| | | | | CASCADE ® | | | | |

TABLE 13
Example of Working Detergent Solutions
Working Detergent Solutions

| | Temp | Detergent | | | |
|---|---|---|---|---|---|
| Detergent | (C.) | g/L | pH | Buffer | Gpg |
| TIDE ® 2X Cold | 16 | 0.98 | 8 | 5 mM | 6 |
| | | | | HEPES | |
| TIDE ® 2X Cold | 32 | 0.98 | 8 | 5 mM | 6 |
| | | | | HEPES | |
| TIDE ® 2X Cold | 16 | 0.98 | 7 | 5 mM | 6 |
| | | | | MOPS | |

Table 14 provides granular laundry detergent compositions produced in accordance with the disclosure suitable for laundering fabrics.

TABLE 14
Granular Laundry Detergent Compositions and Their Components
Granular Laundry Detergent Compositions and Their Components

| | Detergent Compositions | | | | | |
|---|---|---|---|---|---|---|
| Component | 1 | 2 | 3 | 4 | 5 | 6 |
| Linear alkylbenzenesulfonate | 15 | 12 | 20 | 10 | 12 | 13 |
| with aliphatic carbon chain | | | | | | |
| length C₁₁-C₁₂ | | | | | | |
| Other surfactants | 1.6 | 1.2 | 1.9 | 3.2 | 0.5 | 1.2 |
| Phosphate builder(s) | 2 | 3 | 4 | | | |
| Zeolite | | 1 | | 1 | 4 | 1 |
| Silicate | 4 | 5 | 2 | 3 | 3 | 5 |
| Sodium Carbonate | 2 | 5 | 5 | 4 | 0 | 3 |
| Polyacrylate (MW 4500) | 1 | 0.6 | 1 | 1 | 1.5 | 1 |
| Carboxymethyl cellulose | 1 | — | 0.3 | — | 1.1 | — |
| (Finnfix BDA ex CPKelco) | | | | | | |
| Celluclean ® (15.6 mg/g) | 0.23 | 0.17 | 0.5 | 0.2 | 0.2 | 0.6 |
| Lipase (20 mg/g) | 0.2 | | 0.1 | | 0.3 | |
| Stainzyme Plus ® (14 mg/g) | 0.23 | 0.17 | 0.5 | 0.2 | 0.2 | 0.6 |
| Mannaway 4.0T (4 mg/g) | 0.1 | | | 0.1 | | 0.1 |
| Fluorescent Brightener(s) | 0.16 | 0.06 | 0.16 | 0.18 | 0.16 | 0.16 |
| Diethylenetriamine | 0.6 | | 0.6 | 0.25 | 0.6 | 0.6 |
| pentaacetic acid or Ethylene | | | | | | |
| diamine tetraacetic acid | | | | | | |
| MgSO₄ | 1 | 1 | 1 | 0.5 | 1 | 1 |
| Bleach or Bleach activator(s) | 6.88 | | 6.12 | 2.09 | 1.17 | 4.66 |
| Ethoxylated thiophene Hueing | 0.002 | 0.001 | 0.003 | 0.003 | — | — |
| Dye⁵ | | | | | | |
| Direct Violet 9 ex Ciba | | | | 0.0006 | 0.0004 | 0.0006 |
| Specialty Chemicals | | | | | | |

| | |
|---|---|
| Sulfate/Citric Acid/Sodium | Balance to 100% |
| Bicarbonate/Moisture/perfume | |

In Table 14, all enzyme levels expressed as % enzyme raw material, except for lipolytic enzyme (of this disclosure) which is expressed as % of active protein added to the product.

Table 15 provides granular laundry detergent compositions suitable for top-loading automatic washing machines (detergent compositions 7-9) and front loading washing machines (detergent compositions 10-11). The lipolytic enzyme variant tested and/or lipolytic enzyme of the present disclosure can be added separately to these formulations so that the final concentration in the wash liquor is between 0.01 ppm and 10 ppm.

TABLE 15
Granular Laundry Detergent Compositions and Their Components
Granular Laundry Detergent Compositions and Their Components

| | Detergent Composition | | | | |
|---|---|---|---|---|---|
| Component | 7 | 8 | 9 | 10 | 11 |
| Surfactants | | | | | |
| C₁₆₋₁₇ Branched alkyl sulfate | 3.55 | 15.8 | | | |
| C₁₂₋₁₄ alkyl sulphate | | | 1.5 | | |
| Sodium linear alkylbenzenesulfonate | 9.6 | | 10.6 | 7.5 | 9 |
| with aliphatic chain length C₁₁-C₁₂ | | | | | |
| Sodium C_{14/15} alcohol ethoxy-3- | 1.15 | | | 2.88 | |
| sulfate | | | | | |
| Sodium C_{14/15} alkyl sulphate | 2.37 | | | | |
| C_{14/15} alcohol ethoxylate with average | | | | 1.17 | 1 |
| 7 moles of ethoxylation | | | | | |
| mono-C₈₋₁₀ alkyl mono-hydroxyethyl | | | | | 0.45 |
| di-methyl quaternary ammonium | | | | | |
| chloride | | | | | |
| Di methyl hydroxyl ethyl lauryl | | | 0.18 | | |
| ammonium chloride | | | | | |
| Zeolite A | 13.9 | 4.7 | 0.01 | 2.9 | 1.8 |
| Sodium Silicate 1.6.ratio | 4 | 0.2 | | 4 | 4 |
| Sodium Silicate 2.35.ratio | | | 8 | | |
| Citric Acid | | | | 2.5 | 1.4 |
| Sodium tripolyphosphate | | | 5 | | |
| Sodium Carbonate | 24.1 | 30 | 16.9 | 24.4 | 21 |
| Nonanoyloxybenzenesuplhonate | 5.78 | 2.81 | 0.96 | | |
| Oxaziridinium-based bleach booster | | | | 0.03 | 0.017 |
| Tetrasodium S,S,-ethyl | | | | 0.2 | |
| enediaminedisuccinate | | | | | |
| Diethylenetriamine penta (methylene | 0.61 | | | | 0.33 |
| phosphonic acid), heptasodium salt | | | | | |
| Hydroxyethane dimethylene | | | | 0.29 | 0.45 |
| phosphonic acid | | | | | |
| Ethylene diamine tetraacetate | | | 0.27 | | |
| MgSO4 | | | 0.47 | 0.5994 | 0.782 |
| Sodium Percarbonate | 7 | 4.4 | | 15.9 | 19.1 |
| Tetra Acetyl Ethylene Diamine | | | | 3.3 | 4.6 |
| Sodium Perborate Monohydrate | | | 1.2 | | |
| Carboxymethyl cellulose | 0.1 | | 0.17 | 1.69 | 0.23 |
| (e.g. Finnfix BDA ex CPKelco) | | | | | |
| Sodium Acrylic acid/maleic acid co- | 0.0236 | 3.8 | | 2 | 2.5 |
| polymer (70/30) | | | | | |
| Sodium polyacrylate (Sokalan PA30 | 4 | | 0.84 | | |
| CL) | | | | | |
| Terephthalate polymer | | | | 0.23 | |
| Polyethylene glycol/vinyl acetate | | | 0.89 | 0.89 | 0.91 |
| random graft co polymer | | | | | |
| Photobleach- zinc phthalocyanine | | | 0.005 | 0.001 | 0.002 |
| tetrasulfonate | | | | | |
| C.I. Fluorescent Brightener 260 | 0.11 | 0.15 | 0.04 | 0.23 | 0.15 |
| C.I. Fluorescent Brightener 351 | | | 0.1 | | |
| (Tinopal ® CBS) | | | | | |
| Suds suppressor granule | | 0.25 | | 0.07 | 0.04 |
| Hydrophobically modified carboxy | | | 0.019 | 0.028 | |
| methyl cellulose (Finnifix ® SH-1) | | | | | |
| Bentonite | | | 8.35 | | |
| Miscellaneous (Dyes, perfumes, | Balance | Balance | Balance | Balance | Balance |
| process aids, moisture and sodium | | | | | |
| sulphate) | | | | | |

In Table 15, surfactant ingredients can be obtained from any suitable supplier, including but not limited to BASF (e.g., LUTENSOL®), Shell Chemicals, Stepan, Huntsman, and Clariant (e.g., PRAEPAGEN®). Zeolite can be obtained from sources such as Industrial Zeolite. Citric acid and sodium citrate can be obtained from sources such as Jungbunzlauer. Sodium percarbonate, sodium carbonate, sodium bicarbonate and sodium sesquicarbonate can be obtained from sources such as Solvay. Acrylate/maleate copolymers can be obtained from sources such as BASF. Carboxymethylcellulose and hydrophobically modified carboxymethyl cellulose can be obtained from sources such as CPKelco. C.I. Fluorescent Brightener 260 can be obtained from 3V Sigma (e.g., OPTIBLANC®, OPTIBLANC® 2M/G, OPTIBLANC®2MG/LT Extra, or OPTIBLANC® Ecobright. Tetrasodium S,S-ethylenediamine disuccinate can be obtained from sources such as Innospec. Terephthalate co-polymer can be obtained from Clariant (e.g., REPELOTEX SF 2). In addition, 1-Hydroxyethane-1,1-diphosphonic acid can be obtained from Thermphos. Oxaziridinium-based bleach booster has the following structure, where R1=2-butyloctyl, and was produced according to US 2006/0089284A1.

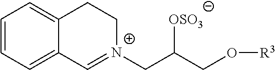

The enzymes NATALASE®, TERMAMYL®, STAINZYME PLUS®, CELLUCLEAN® and MANNAWAY®, can be obtained from Novozymes. Zinc phthalocyanine tetrasulfonate can be obtained from Ciba Specialty Chemicals (e.g., TINOLUX® BMC). Suds suppressor granule can be obtained from Dow Corning. In these detergent compositions, random graft copolymer is a polyvinyl acetate grafted polyethylene oxide copolymer having a polyethylene oxide backbone and multiple polyvinyl acetate side chains. The molecular weight of the polyethylene oxide backbone is about 6000 and the weight ratio of the polyethylene oxide to polyvinyl acetate is about 40 to 60 and no more than 1 grafting point per 50 ethylene oxide units.

Tables 16-18 provide additional granular detergent compositions suitable for washing machines (detergents 36a-n). The lipolytic enzyme of the present disclosure is added separately to these formulations.

TABLE 16
Additional Granular Laundry Detergent Compositions and Their Components
Additional Granular Laundry Detergent Compositions and Their Components

| | Detergent Composition | | | | |
|---|---|---|---|---|---|
| Component | 36a | 36b | 36c | 36d | 36e |
| Surfactants | | | | | |
| C₁₀ Nonionic | | | | 0.1843 | |
| C₁₆₋₁₇ Branched alkyl sulfate | 3.53 | 3.53 | 3.53 | | |
| C₁₂₋₁₄ alkyl sulphate | | | | | |
| Sodium linear alkylbenzenesulfonate | 8.98 | 8.98 | 8.98 | 13.58 | 14.75 |
| with aliphatic chain length C₁₁-C₁₂ | | | | | |
| Sodium C_{14/15} alcohol ethoxy-3- | 1.28 | 1.28 | 1.28 | | |
| sulfate | | | | | |
| Sodium C_{14/15} alkyl sulphate | 2.36 | 2.36 | 2.36 | | |
| C_{14/15} alcohol ethoxylate with average | | | | | |
| 7 moles of ethoxylation | | | | | |
| mono-C₈₋₁₀ alkyl mono-hydroxyethyl | | | | | |
| di-methyl quaternary ammonium | | | | | |
| chloride | | | | | |
| Di methyl hydroxyl ethyl lauryl | | | | 0.1803 | |
| ammonium chloride | | | | | |
| Zeolite A | 15.31 | 15.31 | 15.31 | | 4.47 |
| Bentonite | | | | 8.35 | |
| Sodium Silicate 1.6.ratio | | | | | 0.16 |
| Sodium Silicate 2.0.ratio | 3.72 | 3.72 | 3.72 | 8.41 | |
| Sodium Silicate 2.35.ratio | | | | | |
| Citric Acid | | | | 0.0066 | |
| Sodium tripolyphosphate | | | | 5.06 | |
| Sodium Carbonate | 26.1 | 26.18 | 26.1 | 15.9 | 29.0 |
| Nonanoyloxybenzenesuplhonate | 5.78 | 5.78 | 5.78 | 1.17 | 1.86 |
| Oxaziridinium-based bleach booster | 0.037 | 0.037 | 0.037 | | |
| Tetrasodium S,S,- | | | | | |
| ethylenediaminedisuccinate | | | | | |
| Diethylenetriamine penta (methylene | 0.62 | 0.62 | 0.62 | | |
| phosphonic acid), heptasodium salt | | | | | |
| Hydroxyethane dimethylene | | | | | |
| phosphonic acid | | | | | |
| Ethylene diamine tetraacetate | | | | 0.2701 | |
| MgSO4 | 0.056 | 0.056 | 0.056 | 0.47 | |
| Sodium Percarbonate | | 7.06 | 7.06 | | 3.64 |
| Tetra Acetyl Ethylene Diamine | | | | | |
| Sodium Perborate Monohydrate | | | | 1.47 | |
| Carboxymethyl cellulose | 0.38 | 0.38 | 0.38 | 0.173 | |
| (e.g. Finnfix BDA ex CPKelco) | | | | | |
| Sodium Acrylic acid/maleic acid co- | 3.79 | 3.78 | 3.79 | | 3.64 |
| polymer (70/30) | | | | | |
| Sodium polyacrylate (Sokalan PA30 | 3.78 | 3.78 | 3.78 | 0.842 | |
| CL) | | | | | |
| Terephthalate polymer | | | | | |
| Polyethylene glycol/vinyl acetate | | | | 0.89 | |
| random graft co polymer | | | | | |
| Photobleach- zinc phthalocyanine | | | | | |
| tetrasulfonate | | | | | |
| C.I. Fluorescent Brightener 260 | 0.1125 | 0.1125 | 0.1125 | 0.043 | 0.15 |
| C.I. Fluorescent Brightener 351 | | | | 0.0952 | |
| (Tinopal ® CBS) | | | | | |
| Suds suppressor granule | 0.015 | 0.015 | 0.015 | | 0.031 |
| Hyrdophobically modified carboxy | | | | | |
| methyl cellulose (Finnifix ® SH-1) | | | | | |
| Bentonite | | | | | |
| Miscellaneous (Dyes, perfumes, | Balance | Balance | Balance | Balance | Balance |
| process aids, moisture and sodium | | | | | |
| sulphate) | | | | | |

TABLE 17
Additional Granular Laundry Detergent Compositions and Their Components

| | Detergent Composition | | | | |
|---|---|---|---|---|---|
| Component | 36f | 36g | 36h | 36i | 36j |
| Surfactants | | | | | |
| C₁₀ Nonionic | 0.1142 | 0.2894 | 0.1885 | 0.1846 | 0.1885 |
| C₁₆₋₁₇ Branched alkyl sulfate | | | | | |
| C₁₂₋₁₄ alkyl sulphate | | | | | |
| Sodium linear alkylbenzenesulfonate | 12.94 | 15.69 | 9.01 | 8.42 | 9.51 |
| with aliphatic chain length C₁₁-C₁₂ | | | | | |
| Sodium C_{14/15} alcohol ethoxy-3- | | | | | |
| sulfate | | | | | |
| Sodium C_{14/15} alkyl sulphate | | | | | |
| C_{12/14} alcohol ethoxylate with average | 2.9 | | | | |
| 7 moles of ethoxylation | | | | | |
| C_{12/14} alcohol ethoxylate with average | | | | 2.44 | |
| 3 moles of ethoxylation | | | | | |
| C_{14/15} alcohol ethoxylate with average | | | 0.97 | 1.17 | 0.97 |
| 7 moles of ethoxylation | | | | | |
| mono-C₈₋₁₀ alkyl mono-hydroxyethyl | | | 0.45 | | |
| di-methyl quaternary ammonium | | | | | |
| chloride | | | | | |
| Di methyl hydroxyl ethyl lauryl | | 0.195 | | | 0.45 |
| ammonium chloride | | | | | |
| Zeolite A | 2.01 | 0.39 | 1.83 | 2.58 | 0.59 |
| Sodium Silicate 1.6.ratio | | | 4.53 | 5.62 | 4.53 |
| Sodium Silicate 2.0.ratio | | 10.1 | | | |
| Sodium Silicate 2.35.ratio | 7.05 | | | | |
| Citric Acid | | | 1.4 | 1.84 | 1.0 |
| Sodium tripolyphosphate | | 5.73 | | | |
| Sodium Carbonate | 12.65 | 15.93 | 21.0 | 27.31 | 20.2 |
| Nonanoyloxybenzenesuplhonate | | 1.73 | | | |
| Oxaziridinium-based bleach booster | | | 0.0168 | 0.0333 | 0.024 |
| Tetrasodium S,S,- | | | | | |
| ethylenediaminedisuccinate | | | | | |
| Diethylenetriamine penta (methylene | | | 0.327 | | 0.3272 |
| phosphonic acid), heptasodium salt | | | | | |
| Hydroxyethane dimethylene | | | 0.45 | 0.2911 | 0.45 |
| phosphonic acid | | | | | |
| Ethylene diamine tetraacetate | | 0.28 | | 0.1957 | |
| MgSO4 | | 0.54 | 0.79 | 0.6494 | 0.793 |
| Sodium Percarbonate | | | 19.1 | 15.85 | 22.5 |
| Tetra Acetyl Ethylene Diamine | | | 4.554 | 3.71 | 5.24 |
| Sodium Perborate Monohydrate | | 5.55 | | | |
| Carboxymethyl cellulose | 0.62 | 0.21 | 0.23 | 1.07 | 0.2622 |
| (e.g. Finnfix BDA ex CPKelco) | | | | | |
| Sodium Acrylic acid/maleic acid co- | 0.40 | 2.61 | 2.5 | 2.00 | 1.75 |
| polymer (70/30) | | | | | |
| Sodium polyacrylate (Sokalan PA30 | | | 0.0055 | 0.011 | 0.008 |
| CL) | | | | | |
| Terephthalate polymer | | | | 0.231 | |
| Polyethylene glycol/vinyl acetate | 0.55 | 1.40 | 0.911 | 0.8924 | 0.911 |
| random graft co polymer | | | | | |
| Photobleach- zinc phthalocyanine | | | | | |
| tetrasulfonate | | | | | |
| C.I. Fluorescent Brightener 260 | 0.1174 | 0.048 | 0.1455 | 0.2252 | 0.1455 |
| C.I. Fluorescent Brightener 351 | | 0.1049 | | | |
| (Tinopal ® CBS) | | | | | |
| Suds suppressor granule | | | 0.04 | 0.0658 | 0.04 |
| Hyrdophobically modified carboxy | | | | | |
| methyl cellulose (Finnifix ® SH-1) | | | | | |
| Bentonite | | | | | |
| Miscellaneous (Dyes, perfumes, | Balance | Balance | Balance | Balance | Balance |
| process aids, moisture and sodium | | | | | |
| sulphate) | | | | | |

TABLE 18
Additional Granular Laundry Detergent Compositions and Their Components

| | Detergent Composition | | | | |
|---|---|---|---|---|---|
| Component | 36k | 36l | 36m | 36n | |
| Surfactants | | | | | |
| C₁₀ Nonionic | 0.1979 | 0.1979 | 0.1979 | 0.1979 | |
| C₁₆₋₁₇ Branched alkyl sulfate | | | | | |
| C₁₂₋₁₄ alkyl sulphate | | | | | |
| Sodium linear alkylbenzenesulfonate | 8.92 | 8.92 | 11.5 | 11.5 | |
| with aliphatic chain length C₁₁-C₁₂ | | | | | |
| Sodium C_{14/15} alcohol ethoxy-3- | 1.62 | 1.62 | 1.125 | 1.125 | |
| sulfate | | | | | |
| Sodium C_{14/15} alkyl sulphate | | | | | |
| C_{14/15} alcohol ethoxylate with average | 1.0 | 1.0 | 1.5 | 1.5 | |
| 7 moles of ethoxylation | | | | | |
| mono-C₈₋₁₀ alkyl mono-hydroxyethyl | | | | | |
| di-methyl quaternary ammonium | | | | | |
| chloride | | | | | |
| Di methyl hydroxyl ethyl lauryl | | | | | |
| ammonium chloride | | | | | |
| Zeolite A | 1.63 | 1.63 | 2.0 | 2.0 | |
| Sodium Silicate 1.6.ratio | 4.75 | 4.75 | 4.75 | 4.75 | |
| Sodium Silicate 2.0.ratio | | | 0.06 | 0.06 | |
| Sodium Silicate 2.35.ratio | | | | | |
| Citric Acid | 1.10 | 1.10 | 1.1 | 1.1 | |
| Sodium tripolyphosphate | | | | | |
| Sodium Carbonate | 23.3 | 23.3 | 23.3 | 23.3 | |
| Nonanoyloxybenzenesuplhonate | | | | | |
| Oxaziridinium-based bleach booster | 0.021 | 0.021 | 0.015 | 0.015 | |
| Tetrasodium S,S,- | 0.26 | 0.26 | 0.26 | 0.26 | |
| ethylenediaminedisuccinate | | | | | |
| Diethylenetriamine penta (methylene | | | | | |
| phosphonic acid), heptasodium salt | | | | | |
| Hydroxyethane dimethylene | 0.47 | 0.47 | 0.47 | 0.47 | |
| phosphonic acid | | | | | |
| Ethylene diamine tetraacetate | | | | | |
| MgSO4 | 0.83 | 0.83 | 0.82 | 0.82 | |
| Sodium Percarbonate | 19.35 | 19.35 | 19.35 | 19.35 | |
| Tetra Acetyl Ethylene Diamine | 4.51 | 4.51 | 4.51 | 4.51 | |
| Sodium Perborate Monohydrate | | | | | |
| Carboxymethyl cellulose | 1.01 | 1.01 | 1.01 | 1.01 | |
| (e.g. Finnfix BDA ex CPKelco) | | | | | |
| Sodium Acrylic acid/maleic acid co- | 1.84 | 1.84 | 1.84 | 1.84 | |
| polymer (70/30) | | | | | |
| Sodium polyacrylate (Sokalan PA30 | 0.007 | 0.007 | 0.005 | 0.005 | |
| CL) | | | | | |
| Terephthalate polymer | 0.179 | 0.179 | 0.179 | 0.179 | |
| Polyethylene glycol/vinyl acetate | 0.96 | 0.96 | 0.96 | 0.96 | |
| random graft co polymer | | | | | |
| Photobleach- zinc phthalocyanine | | | | | |
| tetrasulfonate | | | | | |
| C.I. Fluorescent Brightener 260 | 0.153 | 0.153 | 0.171 | 0.171 | |
| C.I. Fluorescent Brightener 351 | | | | | |
| (Tinopal ® CBS) | | | | | |
| Suds suppressor granule | 0.042 | 0.042 | 0.042 | 0.042 | |
| Hyrdophobically modified carboxy | | | | | |
| methyl cellulose (Finnifix ® SH-1) | | | | | |
| Bentonite | | | | | |
| Miscellaneous (Dyes, perfumes, | Balance | Balance | Balance | Balance | Balance |
| process aids, moisture and sodium | | | | | |
| sulphate) | | | | | |

Notes for detergent compositions 36 a-n in Tables 16, 17, and 18: Surfactant ingredients can be obtained from BASF, Ludwigshafen, Germany (Lutensol®); Shell Chemicals, London, UK; Stepan, Northfield, Illinois, USA; Huntsman, Huntsman, Salt Lake City, Utah, USA; Clariant, Sulzbach, Germany (Praepagen®).

Zeolite can be obtained from Industrial Zeolite (UK) Ltd, Grays, Essex, UK.

Ctric acid and sodium citrate can be obtained from Jungbunzlauer, Basel, Switzerland.

Sodium percarbonate, sodium carbonate, sodium bicarbonate and sodium sesquicarbonate can be obtained from Solvay, Brussels, Belgium.

Acrylate/maleate copolymers can be obtained from BASF, Ludwigshafen, Germany.

Carboxymethylcellulose and hydrophobically modified carboxymethyl cellulose can be obtained from CPKelco, Arnhem, The Netherlands.

C.I. Fluorescent Brightener 260 can be obtained from 3V Sigma, Bergamo, Italy as Optiblanc® Optiblanc® 2M/G, Optiblanc® 2MG/LT Extra, or Optiblanc® Ecobright.

Tetrasodium S,S-ethylenediamine disuccinate can be obtained from Innospec, Ellesmere Port, UK.

Terephthalate co-polymer can be obtained from Clariant under the tradename Repelotex SF 2.

1-Hydroxyethane-1,1-diphosphonic acid can be obtained from Thermphos, Vlissingen-Oost, The Netherlands.

Oxaziridinium-based bleach booster has the following structure, where R1=2-butyloctyl, and was produced according to US 2006/0089284A1.

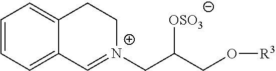

Enzymes Natalase®, Termamyl®, Stainzyme Plus®, Celluclean® and Mannaway®, can be obtained from Novozymes, Bagsvaerd, Denmark.

Zinc phthalocyanine tetrasulfonate can be obtained from Ciba Specialty Chemicals, Basel, Switzerland, as Tinolux® BMC.

Suds suppressor granule can be obtained from Dow Corning, Barry, UK.

Random graft copolymer is a polyvinyl acetate grafted polyethylene oxide copolymer having a polyethylene oxide backbone and multiple polyvinyl acetate side chains. The molecular weight of the polyethylene oxide backbone is about 6000 and the weight ratio of the polyethylene oxide to polyvinyl acetate is about 40 to 60 and no more than 1 grafting point per 50 ethylene oxide units.

In light of the foregoing, it is evident that concentrations of detergent compositions in typical wash solutions throughout the world varies from less than about 300 ppm of detergent composition (“low detergent concentration geographies”) to 10000 ppm in Europe and about 6000 ppm in high suds phosphate builder geographies.

The concentrations of the typical wash solutions are determined empirically. For example, in the U.S., a typical washing machine holds a volume of about 64.4 L of wash solution. Accordingly, in order to obtain a concentration of about 1000 ppm of detergent within the wash solution about 64.4 g of detergent composition must be added to the 64.4 L of wash solution. This amount is the typical amount measured into the wash water by the consumer using the measuring cup provided with the detergent.

As a further example, different geographies use different wash temperatures. The temperature of the wash water in Japan is typically less than that used in Europe. For example, the temperature of the wash water in North America and Japan is typically between about 10 and about 30° C. (e.g., about 20° C.), whereas the temperature of wash water in Europe is typically between about 30 and about 60° C. (e.g., about 40° C.). However, in the interest of saving energy, many consumers are switching to using cold water washing. In addition, in some further regions, cold water is typically used for laundry, as well as dish washing applications. In some embodiments, the “cold water washing” of the present disclosure utilizes “cold water detergent” suitable for washing at temperatures from about 10° C. to about 40° C., or from about 20° C. to about 30° C., or from about 15° C. to about 25° C., as well as all other combinations within the range of about 15° C. to about 35° C., and all ranges within 10° C. to 40° C.

As a further example, different geographies typically have different water hardness. Water hardness is usually described in terms of the grains per gallon mixed Ca²⁺/Mg²⁺. Hardness is a measure of the amount of calcium (Ca²⁺) and magnesium (Mg²⁺) in the water. Most water in the United States is hard, but the degree of hardness varies (Table 19). Moderately hard (60-120 ppm) to hard (121-181 ppm) water has 60 to 181 parts per million (parts per million converted to grains per U.S. gallon is ppm # divided by 17.1 equals grains per gallon) of hardness minerals.

TABLE 19
Water hardness

| Water | Grains per gallon | Parts per million |
|---|---|---|
| Soft | less than 1.0 | less than 17 |
| Slightly hard | 1.0 to 3.5 | 17 to 60 |
| Moderately hard | 3.5 to 7.0 | 60 to 120 |
| Hard | 7.0 to 10.5 | 120 to 180 |
| Very hard | greater than 10.5 | greater than 180 |

European water hardness is typically greater than about 10.5 (for example about 10.5 to about 20.0) grains per gallon mixed Ca²⁺/Mg²⁺ (e.g., about 15 grains per gallon mixed Ca²⁺/Mg²⁺). North American water hardness is typically greater than Japanese water hardness, but less than European water hardness. For example, North American water hardness can be between about 3 to about 10 grains, about 3 to about 8 grains or about 6 grains. Japanese water hardness is typically lower than North American water hardness, usually less than about 4, for example about 3 grains per gallon mixed Ca²⁺/Mg²⁺.

Accordingly, in some embodiments, the present disclosure provides lipolytic enzyme variants that show improved wash performance in at least one set of wash conditions (e.g., water temperature, water hardness, and/or detergent concentration). In some embodiments, the lipolytic enzyme variants of the present disclosure are comparable in wash performance to other lipase lipolytic enzymes. In some embodiments, the lipolytic enzyme variants of the present disclosure exhibit enhanced wash performance as compared to lipase lipolytic enzymes currently commercially available.

In addition, the lipolytic enzyme variants of the present disclosure find use in cleaning compositions that do not include detergents, again either alone or in combination with builders and stabilizers.

In some embodiments, an effective amount of one or more lipolytic enzyme variant(s) provided herein is included in compositions useful for cleaning a items in need of lipid stain removal. Such cleaning compositions include cleaning compositions for such applications as cleaning laundry, hard surfaces (e.g., the hard surface of a table, table top, wall, furniture item, floor, ceiling, medical instrument, examination table, etc.), fabrics, and dishes. Indeed, in some embodiments, the present disclosure provides fabric cleaning compositions, while in other embodiments, the present disclosure provides non-fabric cleaning compositions. It is intended that the present disclosure encompass detergent compositions in any form (i.e., liquid, granular, bar, semi-solid, gels, emulsions, tablets, capsules, etc.).

As indicated above, the cleaning compositions of the present disclosure are formulated into any suitable form and prepared by any process chosen by the formulator, non-limiting examples of which are described in U.S. Pat. Nos. 5,879,584, 5,691,297, 5,574,005, 5,569,645, 5,516,448, 5,489,392, and 5,486,303, all of which are incorporated herein by reference. In some embodiments in which a low pH cleaning composition is desired, the pH of such composition is adjusted via the addition of an acidic material such as HCl.

### Methods of Use

In some embodiments, the cleaning compositions of the present disclosure find use in cleaning surfaces (e.g., dishware), laundry, hard surfaces, contact lenses, fabric, etc. In some embodiments, at least a portion of the surface is contacted with at least one embodiment of the cleaning compositions of the present disclosure, in neat form or diluted in a wash liquor, and then the surface is optionally washed and/or rinsed.

For purposes of the present disclosure, “washing” includes, but is not limited to, scrubbing, and mechanical washing. In some embodiments, the cleaning compositions of the present disclosure are used at concentrations of from about 300 ppm to about 15,000 ppm in solution. In some embodiments in which the wash solvent is water, the water temperature typically ranges from about 5° C. to about 90° C.

The compositions of the present disclosure also find use in detergent additive products in solid or liquid form. Such additive products are intended to supplement and/or boost the performance of conventional detergent compositions and can be added at any stage of the cleaning process.

The present disclosure provides methods for cleaning or washing an item or surface (e.g., hard surface) in need of cleaning, including, but not limited to methods for cleaning or washing a dishware item, a tableware item, a fabric item, a laundry item, personal care item, etc., or the like, and methods for cleaning or washing a hard or soft surface (e.g., a hard surface of an item).

In some embodiments, the present disclosure provides methods for washing including manual dishwashing, hard surface cleaning and automatic dishwashing.

In some embodiments, the present disclosure provides a method for cleaning an item, object, or surface in need of cleaning, the method comprising contacting the item or surface (or a portion of the item or surface desired to be cleaned) with at least one variant lipase lipolytic enzyme of the present disclosure or a composition of the present disclosure for a sufficient time and/or under conditions suitable and/or effective to clean the item, object, or surface to a desired degree. Some such methods further comprise rinsing the item, object, or surface with water. For some such methods, the cleaning composition is a dishwashing detergent composition and the item or object to be cleaned is a dishware item or tableware item. As used herein, a “dishware item” is an item generally used in serving or eating food. A dishware item can be, but is not limited to for example, a dish, plate, cup, bowl and the like. As used herein, “tableware” is a broader term that includes, but is not limited to for example, dishes, cutlery, knives, forks, spoons, chopsticks, glassware, pitchers, sauce boats, drinking vessels, serving items, etc. It is intended that “tableware item” includes any of these or similar items for serving or eating food. For some such methods, the cleaning composition is an automatic dishwashing detergent composition or a hand dishwashing detergent composition and the item or object to be cleaned is a dishware or tableware item. For some such methods, the cleaning composition is a laundry detergent composition (e.g., a power laundry detergent composition or a liquid laundry detergent composition), and the item to be cleaned is a fabric item. In some other embodiments, the cleaning composition is a laundry pre-treatment composition.

In some embodiments, the present disclosure provides methods for cleaning or washing a fabric item optionally in need of cleaning or washing, respectively. In some embodiments, the methods comprise providing a composition comprising the lipolytic enzyme variant, including but not limited to fabric or laundry cleaning composition, and a fabric item or laundry item in need of cleaning, and contacting the fabric item or laundry item (or a portion of the item desired to be cleaned) with the composition under conditions sufficient or effective to clean or wash the fabric or laundry item to a desired degree.

In some embodiments, the present disclosure provides a method for cleaning or washing an item or surface (e.g., hard surface including the hard surface of a table, table top, wall, furniture item, floor, ceiling, medical instrument, examination table, etc.) optionally in need of cleaning, the method comprising providing an item or surface to be cleaned or washed and contacting the item or surface (or a portion of the item or surface desired to be cleaned or washed) with at least one lipase variant of the disclosure or a composition of the disclosure comprising at least one such lipase variant for a sufficient time and/or under conditions sufficient or effective to clean or wash the item or surface to a desired degree. Such compositions include, but are not limited to for example, a cleaning composition or detergent composition of the disclosure (e.g., a hand dishwashing detergent composition, hand dishwashing cleaning composition, laundry detergent or fabric detergent or laundry or fabric cleaning composition, liquid laundry detergent, liquid laundry cleaning composition, powder laundry detergent composition, powder laundry cleaning composition, automatic dishwashing detergent composition, laundry booster cleaning or detergent composition, laundry cleaning additive, and laundry pre-spotter composition, etc.). In some embodiments, the method is repeated one or more times, particularly if additional cleaning or washing is desired. For example, in some instance, the method optionally further comprises allowing the item or surface to remain in contact with the at least one lipolytic enzyme variant or composition for a period of time sufficient or effective to clean or wash the item or surface to the desired degree. In some embodiments, the methods further comprise rinsing the item or surface with water and/or another liquid. In some embodiments, the methods further comprise contacting the item or surface with at least one lipolytic enzyme variant of the disclosure or a composition of the disclosure again and allowing the item or surface to remain in contact with the at least one lipolytic enzyme variant or composition for a period of time sufficient to clean or wash the item or surface to the desired degree. In some embodiments, the cleaning composition is a dishwashing detergent composition and the item to be cleaned is a dishware or tableware item. In some embodiments of the present methods, the cleaning composition is an automatic dishwashing detergent composition or a hand dishwashing detergent composition and the item to be cleaned is a dishware or tableware item. In some embodiments of the methods, the cleaning composition is a laundry detergent composition and the item to be cleaned is a fabric item.

The present disclosure also provides methods of cleaning a tableware or dishware item in an automatic dishwashing machine, the method comprising providing an automatic dishwashing machine, placing an amount of an automatic dishwashing composition comprising at least one lipase variant of the present disclosure or a composition of the disclosure sufficient to clean the tableware or dishware item in the machine (e.g., by placing the composition in an appropriate or provided detergent compartment or dispenser in the machine), putting a dishware or tableware item in the machine, and operating the machine so as to clean the tableware or dishware item (e.g., as per the manufacturer's instructions). In some embodiments, the methods include any automatic dishwashing composition described herein, which comprises, but is not limited to at least one lipase variant provided herein. The amount of automatic dishwashing composition to be used can be readily determined according to the manufacturer's instructions or suggestions and any form of automatic dishwashing composition comprising at least one lipolytic enzyme variant of the disclosure (e.g., liquid, powder, solid, gel, tablet, etc.), including any described herein, may be employed.

The present disclosure also provides methods for cleaning a surface, item or object optionally in need of cleaning, the method comprises contacting the item or surface (or a portion of the item or surface desired to be cleaned) with at least one variant lipase of the present disclosure or a cleaning composition of the disclosure in neat form or diluted in a wash liquor for a sufficient time and/or under conditions sufficient or effective to clean or wash the item or surface to a desired degree. The surface, item, or object may then be (optionally) washed and/or rinsed if desired. For purposes of the present disclosure, “washing” includes, but is not limited to for example, scrubbing and mechanical agitation. In some embodiments, the cleaning compositions are employed at concentrations of from about 500 ppm to about 15,000 ppm in solution (e.g., aqueous solution). When the wash solvent is water, the water temperature typically ranges from about 5° C. to about 90° C. and when the surface, item or object comprises a fabric, the water to fabric mass ratio is typically from about 1:1 to about 30:1.

The present disclosure also provides methods of cleaning a laundry or fabric item in an washing machine, the method comprising providing an washing machine, placing an amount of a laundry detergent composition comprising at least one variant lipase of the disclosure sufficient to clean the laundry or fabric item in the machine (e.g., by placing the composition in an appropriate or provided detergent compartment or dispenser in the machine), placing the laundry or fabric item in the machine, and operating the machine so as to clean the laundry or fabric item (e.g., as per the manufacturer's instructions). The methods of the present disclosure include any laundry washing detergent composition described herein, comprising but not limited to at least one of any variant lipase provided herein. The amount of laundry detergent composition to be used can be readily determined according to manufacturer's instructions or suggestions and any form of laundry detergent composition comprising at least one lipolytic enzyme variant of the disclosure (e.g., solid, powder, liquid, tablet, gel, etc.), including any described herein, may be employed.

The present disclosure also provides variants, such as lipase enzyme variants useful for pulp and paper processing, including controlling organic contaminants in fibers. The fiber can be cellulose fibers and in some instances are recycled fibers from a variety of paper products or fiber containing products, such as old corrugated containers (OCC), old newsprint (ONP), mixed office waste (MOW), or combinations thereof. These types of paper containing products typically contain large amounts of organic contaminants which are present in the paper products. When these types of paper products are recycled, these organic contaminants are present along with the fibers formed during the pulping stage of a papermaking process. These organic contaminants, if not substantially removed, can severely interfere with subsequent stages in the papermaking process by affecting the quality of the resulting sheets of paper formed and/or affecting the machinery used to form the paper. Accordingly, the removal of such organic contaminants is important to the paper making process when such organic contaminants are present in fibers.

For purposes of the present disclosure, examples of organic contaminants include what is known in the industry as “stickies” and include, but are not limited to, synthetic polymers resulting from adhesives and the like, glues, hot melts, coatings, coating binders, ink residues, de-inking chemicals, wood resins, rosin, and unpulped wet strength resins. These types of materials are typically found in paper containing products, such as newsprint, corrugated container, and/or mixed office waste. These organic contaminants typically will have polymers present, such as styrene butadiene rubber, vinyl acrylates, polyisoprene, polybutadiene, natural rubber, ethyl vinyl acetates, polyvinyl acetates, ethylvinyl alcohols, polyvinyl alcohols, styrene acrylates, and other synthetic type polymers.

In the process of the present disclosure, these organic contaminants are controlled by contacting the fiber containing the organic contaminants with a composition containing at least one variant of the present disclosure for a sufficient time and in a sufficient amount to control the organic contaminants present in the fiber. The compositions of the present disclosure preferably disperse or convert the organic contaminants to organic species that do not affect the paper making process. For instance, the polyvinyl acetates are preferably dispersed and/or converted to polyvinyl alcohols, which do not affect the papermaking process. This preferred manner that the compositions achieve control of organic contaminants is quite different from collecting contaminants by flotation.

For purposes of the present disclosure, controlling organic contaminants present in fibers having organic contaminants is understood as one or more of the following: reducing the size of contaminant particles, reducing the number or amount of measurable particles present, and/or reducing the tackiness of the organic contaminants. In some embodiments, when controlling organic contaminants using the methods of the present disclosure, all of these reductions occur. In some embodiments, the reduction of the size of contaminant particles is by at least about 5%, or by from about 10% to about 75% as compared to when no variant of the present disclosure is present. Similarly, the reduction in the number or amount of organic contaminants present in the fiber is reduced by at least about 5%, or by from about 10% to about 75% when compared to fibers which have not been treated with a variant of the present disclosure. Also, the reduction of tackiness of the organic contaminants can be reduced by at least about 5%, or by from about 10% to about 75% when compared to fibers which have not been treated with a variant of the present disclosure.

The compositions containing at least one variant of the present disclosure can also contain as an option other conventional paper treatment chemicals or ingredients such as, but not limited to, surfactants, solvents, suspension aids, fillers, chelants, preservatives, buffers, water, stabilizers, and the like. These additional ingredients can be present in conventional amounts.

In some embodiments of the disclosure, a method is provided for treating polyester, including clean, unsoiled polyester, comprising contacting said polyester textile with an enzyme solution having variant of the present disclosure for a time and under conditions such that the properties of the polyester are modified. Preferably, the polyester is a fiber, yarn, fabric or finished textile product comprising such fiber, yarn or fabric. Further preferably, the properties that are modified comprise those such as improved hand, feel and/or weight of a textile made from such fiber, yarn or article. In some embodiments, the present disclosure is to provide for a mechanism to modify the textile characteristics of a polyester comprising textile. Thus, in this embodiment of the disclosure, it is often advantageous to apply the polyesterase to textile products which are unsoiled, i.e., do not comprise stains which are typically subjected to commercial laundry detergents. In other embodiments, the present disclosure is to provide for a method of laundering stains from polyester fabrics.

In another embodiment of the disclosure, a method is provided for treating a polyester fiber, yarn or fabric, prior to its incorporation into a textile product or the application of a textile finish with an enzyme variant of the present disclosure for a time and under conditions such that the properties of the polyester are modified. Accordingly, in the embodiment wherein textile components are treated separately, the treated polyester components (i.e., fibers, yarns, fabrics), can be incorporated into a textile product through standard methods for producing polyester textiles, e.g., processes such as weaving, sewing and cutting and stitching, thus conferring the modifications to the finished textile product.

In yet another method embodiment of the disclosure, a method is provided for treating a polyester resin or film with an enzyme variant of the present disclosure for a time and under conditions such that the properties of the polyester are modified. The treated polyester may be a finished resin or film product or may be incorporated into a product through, for example, mechanical construction, thus conferring the modifications to the finished textile product.

In yet another method embodiment of the disclosure, a polyester waste product is treated with an enzyme variant of the present disclosure to degrade the polyester waste product to easily dispose of or recycled compounds. This embodiment is particularly useful in the degradation of polyester based plastics which are becoming increasingly problematic in waste disposal and dumping. An alternative of this embodiment is that the present disclosure may be used to increase the amount of microbially digestible material in a waste product so as to facilitate complete degradation or composting of such waste.

In the method according to the disclosure, the solution containing an enzyme variant of the present disclosure as provided herein is contacted with the polyester fiber, yarn, fabric or textile which comprises such fiber, yarn or fabric under conditions suitable for the enzyme to exhibit polyester modification. The present disclosure is preferably directed to the use of the polyesterase in the manufacture of the textile product, and not necessarily in combination with a detergent for the purpose of removing stains which occur during wear. Thus, in this embodiment, the application of the enzyme variant of the present disclosure to the polyester article occurs prior to spinning of the fiber into a yarn, prior to the incorporation of the yarn into a fabric and/or prior to the construction of the textile product which comprises the polyester. However, it is within the present disclosure as well, and also a preferred embodiment hereon, to treat the completed textile product with the enzyme variant of the present disclosure identified herein.

Non-limiting examples of compositions and methods disclosed herein are as follows:

1. A lipolytic enzyme variant or an active fragment thereof comprising an amino acid modification to a parent lipolytic enzyme, wherein the amino acid modification is at a position of the lipolytic enzyme variant selected from the group consisting of 4, 29, 64, 70, 95, 122, 146, 159, 164, 178, 179, 203, 205, 206, 209, 212, 227, 267, 268, 272 and 280, wherein the amino acid positions of the variant are numbered by correspondence with the amino acid sequence of SEQ ID NO: 2, wherein the lipolytic enzyme variant has at least 60% sequence identity to SEQ ID NO: 1.

2. The lipolytic enzyme variant of embodiment 1, wherein said variant is derived from a parent lipolytic enzyme having 60%, 65%, 70%, 75%, 80%, 85%, 86%, 87%, 88%, 89%, 90%, 91%, 92%, 93%, 94%, 95%, %, 97%, 98%, 99%, or 100% amino acid sequence identity to the amino acid sequence of SEQ ID NO: 1.

3. The lipolytic enzyme variant of embodiment 1, wherein said variant comprises an amino acid sequence having 60%, 65%, 70%, 75%, 80%, 85%, 86%, 87%, 88%, 89%, 90%, 91%, 92%, 93%, 94%, 95%, %, 97%, 98% or 99% amino acid sequence identity to the amino acid sequence of SEQ ID NO: 1.

4. The lipolytic enzyme variant of any preceding embodiments, wherein the amino acid modification is a substitution selected from the group consisting of T4K, A29R, I64V, V70A, S95N, D122P, N146H, A159D, A159S, A178K, 1179K, N164D, N164R, L203R, L203P, A205P, G206D, G206E, G209A, G209D, L212R, S227T, R267P, G268K, I272V and N280K.

5. The lipolytic enzyme variant or active fragment thereof of any one of embodiments 1-4, wherein said lipolytic enzyme variant or active fragment thereof has an improved performance relative to the parent lipolytic enzyme, wherein the improved performance is selected from the group consisting of an improved wash performance, a decreased malodor, an increased detergent stability, an increased thermostability, an increased calcium ion binding stability, an increased protease stability, or any one combination thereof.

6. The lipolytic enzyme variant or active fragment thereof of embodiment 5, wherein the improved wash performance is an improved wash performance at a low temperature.

7. The lipolytic enzyme variant or active fragment thereof of embodiment 5, wherein the variant or active fragment has a wash performance index (PI(wash)) relative to the parent lipolytic enzyme that is greater than 1.0.

8. The lipolytic enzyme variant or active fragment thereof of embodiment 5, wherein the variant or active fragment has a thermostability performance index (PI(thermostability)) relative to the parent lipolytic enzyme that is greater than 1.0.

9. The lipolytic enzyme variant or active fragment thereof of embodiment 5, wherein the variant or active fragment has a calcium ion stability index (PI(calcium ion binding stability)) relative to the parent lipolytic enzyme that is greater than 1.0.

10. The lipolytic enzyme variant or active fragment thereof of embodiment 5, wherein the variant or active fragment has a protease stability that is greater that the protease stability of the parent lipolytic enzyme.

11. The lipolytic enzyme variant or active fragment thereof of any one of embodiment 1-4, wherein the variant or active fragment has a decreased malodor performance index (PI(malodor) relative to the lipolytic enzyme of SEQ ID NO: 7 that is less than 1.0.

12. The lipolytic enzyme variant or active fragment thereof of any preceding embodiment, wherein the variant or active fragment has lipolytic activity.

13. A composition comprising at least one lipolytic enzyme variant or fragment according to any preceding embodiment.

14. The composition according to embodiment 13, wherein said composition is a detergent composition.

15. The composition according to embodiment 14, wherein said detergent composition is selected from a laundry detergent, a fabric softening detergent, a dishwashing detergent, and a hard-surface cleaning detergent.

16. The composition of any one of embodiments 13-15, wherein said composition further comprises one or more calcium ions and/or zinc ions; one or more enzyme stabilizers; from about 0.001% to about 1.0 weight % of said lipolytic enzyme variant(s); one or more bleaching agents; one or more adjunct materials; and/or one or more additional enzymes or enzyme derivatives selected from the group consisting of acyl transferases, alpha-amylases, beta-amylases, alpha-galactosidases, arabinosidases, aryl esterases, beta-galactosidases, carrageenases, catalases, cellobiohydrolases, cellulases, chondroitinases, cutinases, DNase or nuclease, endo-beta-1,4-glucanases, endo-beta-mannanases, esterases, exo-mannanases, galactanases, glucoamylases, hemicellulases, hyaluronidases, keratinases, laccases, lactases, ligninases, lipases, lipoxygenases, lysozymes, mannanases, oxidases, pectate lyases, pectin acetyl esterases, pectinases, pentosanases, perhydrolases, peroxidases, phenoloxidases, phosphatases, phospholipases, phytases, polygalacturonases, proteases, pullulanases, reductases, rhamnogalacturonases, beta-glucanases, tannases, transglutaminases, xylan acetyl-esterases, xylanases, xyloglucanases, xylosidases, metalloproteases, nucleases, additional serine proteases, and combinations thereof.

17. The composition of any one of embodiments 13-16, wherein said composition is a granular, powder, solid, bar, liquid, tablet, gel, paste or unit dose composition.

18. A DNA sequence encoding the lipolytic enzyme variant or active fragment of embodiment 1.

19. An expression vector or cassette comprising the DNA sequence of embodiment 18.

20. The expression vector or cassette of embodiment 19, wherein the DNA sequence is operably linked to a promoter.

21. A recombinant host cell comprising the DNA sequence of embodiment 18.

22. Use of the lipolytic enzyme variant or fragment of any of embodiments 1-12 for hydrolyzing a lipolytic enzyme substrate.

23. A method for cleaning a surface comprising contacting the surface with a composition comprising at least one lipolytic enzyme variant of any of embodiments 1-12, and optionally a surfactant.

24 A method of producing a lipolytic enzyme variant comprising culturing the host cell comprising a DNA sequence encoding the lipolytic enzyme variant or active fragment of embodiment 1 in a culture medium under conditions conducive for the production of the lipolytic enzyme variant and recovering the lipolytic enzyme variant from said culture medium.

25. A lipolytic enzyme variant or an active fragment thereof comprising an amino acid modification to a parent lipolytic enzyme, wherein the amino acid modification is at a position of the lipolytic enzyme variant selected from the group consisting of 2, 4, 11, 13, 14, 17, 18, 19, 20, 22, 27, 28, 29, 30, 31, 33, 43, 44, 46, 47, 49, 50, 52, 53, 57, 60, 62, 64, 65, 67, 68, 70, 83, 85, 91, 93, 95, 99, 105, 117, 118, 119, 120, 121, 122, 125, 127, 128, 130, 131, 132, 134, 135, 137, 138, 140, 141, 146, 147, 150, 154, 155, 159, 160, 164, 166, 170, 171, 175, 178, 179, 180, 185, 189, 196, 199, 203, 204, 205, 206, 208, 209, 212, 217, 218, 221, 223, 224, 227, 229, 247, 251, 252, 255, 258, 261, 262, 264, 267, 268, 272 and 280, wherein the amino acid positions of the variant are numbered by correspondence with the amino acid sequence of SEQ ID NO: 2, wherein the lipolytic enzyme variant has at least 60% sequence identity to SEQ ID NO: 2.

26. The lipolytic enzyme variant of embodiment 25, wherein said variant is derived from a parent lipolytic enzyme having 60%, 65%, 70%, 75%, 80%, 85%, 86%, 87%, 88%, 89%, 90%, 91%, 92%, 93%, 94%, 95%, %, 97%, 98%, 99%, or 100% amino acid sequence identity to the amino acid sequence of SEQ ID NO: 2.

27. The lipolytic enzyme variant of embodiment 25, wherein said variant comprises an amino acid sequence having 60%, 65%, 70%, 75%, 80%, 85%, 86%, 87%, 88%, 89%, 90%, 91%, 92%, 93%, 94%, 95%, 96%, 97%, 98% or 99% amino acid sequence identity to the amino acid sequence of SEQ ID NO: 2.

28. The lipolytic enzyme variant of any preceding embodiments, wherein the amino acid modification is a substitution selected from the group consisting of S002V, T4K, H011A, H011K, L013C, L013F, L013W, S014C, S014G, S014L, S014W, D017A, D017G, D017S, D017T, D018A, D018F, D018G, D018I, D018K, D018L, D018M, D018N, D018P, D018R, D018T, D018V, D018W, 1019C, V020G, Y022F, Y022I, Y022V, Y022W, G027K, I028V, A29R, D030A, D030K, D030R, A031G, E033I, E033K, S043K, S043R, L044Q, A046G, A046S, A046T, F047C, F047Y, S049D, S049T, N050P, V052F, V052K, V052M, R053I, R053M, R053Q, R053T, R053V, L057C, F060K, Q062A, 164V, L0651H, E067I, E067Q, E067R, E067S, E067T, T068S, A070V, A070Y, L083M, C085A, C085G, K091D, K091E, K091N, K091Q, A093E, S095P, S095N, V099I, V105A, V105C, V105P, R117A, R117C, R117D, R117E, R117G, R117I, R117K, R117N, R117Q, R117S, R117T, R117V, I118D, I118E, I118G, I118H, I118N, I118S, M119C, M119V, M119Y, R120A, R120C, R120E, R120F, R120G, R120H, R120I, R120K, R120L, R120M, R120Q, R120T, R120V, R120W, K121A, K121E, K121N, K121Q, K121S, K121T, D122P, P125D, P125E, P125F, Y127C, Y127D, Y127E, Y127G, Y127H, Y127N, I128A, I128C, I128G, I128K, I128M, I128N, I128Q, I128T, I128Y, A129S, D130C, A131D, A131E, A131N, A131P, A131Q, A131S, A131T, V132T, K134C, K134D, K134E, K134F, K134G, K134I, K134M, K134N, K134Q, K134S, K134T, K134V, K134W, K134Y, A135E, G137R, T138I, T138V, 1140E, I140F, I140T, I140V, S141M, N146M, N146H, R147P, P150D, P150E, P150T, I154M, I154N, I154Q, I154T, I154V, A155E, A155M, A159D, A159S, L160A, L160C, L160S, L160T, L160V, N164A, N164E, N164Q, N164S, N164R, N164D, M166A, M166D, M166E, K170A, K170D, K170H, K170Q, K170T, K171C, K171D, K171E, K171Q, G175A, A178D, A178K, I179E, I179K, R180E, R180P, K185C, K185D, K185E, K185L, K185S, N189D, F196I, Y199F, L203C, L203K, L203N, L203P, L203R, L203S, L203T, I204Y, A205C, A205F, A205I, A205L, A205M, A205P, A205V, A205W, A205Y, G206C, G206D, G206E, G206Q, K208E, K208G, K208M, K208Q, G209A, G209C, G209D, G209E, G209Q, L212K, L212R, A217F, A217I, A217L, A217V, A217Y, A218C, V221F, V221T, S223G, A224G, A224H, A224K, A224Q, A224R, A224S, A224W, A224Y, S227D, S227I, S227N, S227T, S227V, R229A, R229C, R229D, R229E, R229G, R229H, R229I, R229K, R229L, R229M, R229N, R229P, R229Q, R229S, R229T, R229V, K247R, A251P, E252Q, L255C, V258I, V261L, A262C, A262F, A262L, A262M, L264K, R267P, G268D, G268K, I272G, I272K, I272P, I272R, I272V and N280K,

29. The lipolytic enzyme variant or active fragment thereof of any one of embodiments 25-28, wherein said lipolytic enzyme variant or active fragment thereof has an improved performance relative to the parent lipolytic enzyme, wherein the improved performance is selected from the group consisting of an improved wash performance, a decreased malodor, an increased detergent stability, an increased thermostability, an increased calcium ion binding stability, an increased protease stability, and any one combination thereof.

30. The lipolytic enzyme variant or active fragment thereof of embodiment 29, wherein the improved wash performance is an improved wash performance at a low temperature.

31. The lipolytic enzyme variant or active fragment thereof of embodiment 29, wherein the variant or active fragment has a wash performance index (PI(wash)) relative to the parent lipolytic enzyme that is greater than 1.0.

32. The lipolytic enzyme variant or active fragment thereof of embodiment 29, wherein the variant or active fragment has a detergent stability index (PI(detergent stability)) relative to the parent lipolytic enzyme that is greater than 1.0.

33. The lipolytic enzyme variant or active fragment thereof of embodiment 29, wherein the variant or active fragment has a thermostability performance index (PI(thermostability)) relative to the parent lipolytic enzyme that is greater than 1.0.

34. The lipolytic enzyme variant or active fragment thereof of embodiment 29, wherein the variant or active fragment has a calcium ion binding stability index (PI(calcium ion binding stability)) relative to the parent lipolytic enzyme that is greater than 1.0.

35. The lipolytic enzyme variant or active fragment thereof of embodiment 29, wherein the variant or active fragment has a protease stability that is greater that the protease stability of the parent lipolytic enzyme.

36. The lipolytic enzyme variant or active fragment thereof of any of embodiment 25-28, wherein the variant or active fragment has a decreased malodor performance index (PI(malodor) relative to the lipolytic enzyme of SEQ ID NO: 7 that is less than 1.0.

37. The lipolytic enzyme variant or active fragment thereof of any preceding embodiments, wherein the variant or active fragment has lipolytic activity.

38. A composition comprising at least one lipolytic enzyme variant or fragment according to any preceding embodiment.

39. The composition according to embodiment 38, wherein said composition is a detergent composition.

40. The composition according to embodiment 39, wherein said detergent composition is selected from a laundry detergent, a fabric softening detergent, a dishwashing detergent, and a hard-surface cleaning detergent.

41. The composition of any one of embodiments 38-40, wherein said composition further comprises one or more calcium ions and/or zinc ions; one or more enzyme stabilizers; from about 0.001% to about 1.0 weight % of said lipolytic enzyme variant(s); one or more bleaching agents; one or more adjunct materials; and/or one or more additional enzymes or enzyme derivatives selected from the group consisting of acyl transferases, alpha-amylases, beta-amylases, alpha-galactosidases, arabinosidases, aryl esterases, beta-galactosidases, carrageenases, catalases, cellobiohydrolases, cellulases, chondroitinases, cutinases, DNase or nuclease, endo-beta-1,4-glucanases, endo-beta-mannanases, esterases, exo-mannanases, galactanases, glucoamylases, hemicellulases, hyaluronidases, keratinases, laccases, lactases, ligninases, lipases, lipoxygenases, lysozymes, mannanases, oxidases, pectate lyases, pectin acetyl esterases, pectinases, pentosanases, perhydrolases, peroxidases, phenoloxidases, phosphatases, phospholipases, phytases, polygalacturonases, proteases, pullulanases, reductases, rhamnogalacturonases, beta-glucanases, tannases, transglutaminases, xylan acetyl-esterases, xylanases, xyloglucanases, xylosidases, metalloproteases, nucleases, additional serine proteases, and combinations thereof.

42. The composition of any one of embodiments 38-41, wherein said composition is a granular, powder, solid, bar, liquid, tablet, gel, paste or unit dose composition.

43. A DNA sequence encoding the lipolytic enzyme variant or active fragment of embodiment 25.

44. An expression vector or cassette comprising the DNA sequence of embodiment 43.

45. The expression vector or cassette of embodiment 20, wherein the DNA sequence is operably linked to a promoter.

46. A recombinant host cell comprising the DNA sequence of embodiment 43.

47. Use of the lipolytic enzyme variant or fragment of any of embodiments 25-37 for hydrolyzing a lipolytic enzyme substrate.

48. A method for cleaning a surface comprising contacting the surface with a composition comprising at least one lipolytic enzyme variant of any of embodiments 25-37, and optionally a surfactant.

49 A method of producing a lipolytic enzyme variant comprising culturing the host cell comprising a DNA sequence encoding the lipolytic enzyme variant or active fragment of embodiment 1 or 25 in a culture medium under conditions conducive for the production of the lipolytic enzyme variant and recovering the lipolytic enzyme variant from said culture medium.

50. The lipolytic enzyme variant or active fragment thereof of embodiment 1, wherein the variant or active fragment thereof comprises amino acid modifications selected from the group consisting of D122P_A205P, T4K_L212R, T4K_L203R, I179K_L212R, A178K_G268K, N164R_A159D, N146H_I179K, N146H_N280K, G206D_G209A, G206E_G209D, G206E_G209A, A159D_G206E_G209D, T4K_L203R_L212R_G268K, A178K_I179K_L203R_L212R, N146H_A178K_I179K_G268K, N146H_A178K_I179K_L212R, T4K_N146H_I179K_L203R_L212R_N280K, T4K_N146H_A178K_L203R_G268K_N280K, A29R_I64V_V70A_S95N_S227T_I272V, A29R_I64V_V70A_S95N_S227T_R267P_I272V A29R_I64V_V70A_S95N_S227T_R267P_I272V, T4K_N146H_A159D_I179K_L203R_L212R_N280K, T4K_N146H_I179K_L203R_G206E_G209D_L212R_N280K, and T4K_N146H_A159D_I179K_L203R_G206E_G209D_L212R_N280K, wherein the amino acid positions of the variant are numbered by correspondence with the amino acid sequence set forth in SEQ ID NO:2, wherein the lipolytic enzyme variant has at least 60% sequence identity to SEQ ID NO: 1.

51. The lipolytic enzyme variant or active fragment thereof of embodiment 25, wherein the variant or active fragment thereof comprises amino acid modifications selected from the group consisting of D122P_A205P, T4K_L212R, T4K_L203R, I179K_L212R, A178K_G268K, N164R_A159D, N146H_I179K, N146H_N280K, G206D_G209A, G206E_G209D, G206E_G209A, A159D_G206E_G209D, T4K_L203R_L212R_G268K, A178K_I179K_L203R_L212R, N146H_A178K_I179K_G268K, N146H_A178K_I179K_L212R, T4K_N146H_I179K_L203R_L212R_N280K, T4K_N146H_A178K_L203R_G268K_N280K, A29R_I64V_S95N_S227T_I272V, A29R_I64V_S95N_S227T_R267P_I272V A29R_I64V_S95N_S227T_R267P_I272V, T4K_N146H_A159D_I179K_L203R_L212R_N280K, T4K_N146H_I179K_L203R_G206E_G209D_L212R_N280K, and T4K_N146H_A159D_I179K_L203R_G206E_G209D_L212R_N280K, wherein the amino acid positions of the variant are numbered by correspondence with the amino acid sequence set forth in SEQ ID NO:2, wherein the lipolytic enzyme variant has at least 60% sequence identity to SEQ ID NO: 2

### EXAMPLES

In the following Examples, unless otherwise stated, parts and percentages are by weight and degrees are Celsius. It should be understood that these Examples, while indicating embodiments of the disclosure, are given by way of illustration only. From the above discussion and these Examples, one skilled in the art can make various changes and modifications of the disclosure to adapt it to various usages and conditions. Such modifications are also intended to fall within the scope of the appended claims.

### Example 1

### Lipase Variants

Synthetic genes encoding a parent lipase amino acid sequence derived from Proteus sp. H24, set forth in SEQ ID NO: 1, and lipase variants thereof were designed for expression in Bacillus subtilis. These genes were cloned and expressed in Bacillus subtilis and their expression was subsequently evaluated.

Lipase titers (enzyme concentration) were determined based on antibody association rates measured by Biolayer Interferometry using the Octet HTX instrument (ForteBio). Biolayer interferometry measures changes in the absorption of visible light at the surface of an optical probe caused by the accumulation of protein biolayer. To measure lipase concentrations in crude Bacillus subtilis supernatants we obtained streptavidin-coated biosensors (ForteBio), and functionalized them with a biotin-labeled polyclonal antibody that recognize lipase variants of SEQ ID NO:1. Polyclonal antibodies were isolated from serum extracted from rabbits inoculated with purified native protein from a lipase variant of SEQ ID NO:1, referred to as A29R_I64V_V70A_S95N_S227T_R267P_I272V, (ProSci). Lipase reactive sensors prepared in this format facilitate the specific accumulation of protein from SEQ ID NO:1 and related variants at the sensor surface when submerged in solutions containing known and unknown concentrations of lipase. Association rates from a SEQ ID NO:1 dilution series of pre-determined concentrations were fit to a four-parameter linear equation using ForteBio Data Analysis 10.0 software (ForteBio) and the equations were used to calculate titer values from the association rates measured in supernatants containing unknown amounts of SEQ ID NO: 1 lipase and variants thereof.

Bacillus subtilis fermentate UFC was produced by standard methods and was filtered through a 0.2 um filter and dialyzed into 10 mM Potassium Phosphate, 0.01% Triton X-100, pH 7.0. Sample was purified using Cation Exchange Chromatography with an Akta Pure (GE Healthcare Life Sciences). In detail, dialyzed material was loaded onto a HiPrep 16/60 SP HP at a 3 mL/min flowrate, and eluted using a 30% gradient over 7 column volumes with 10 mM Potassium Phosphate, 1M NaCl pH 7.0. Elution was collected in 4 mL fractions and pooled based on SDS-PAGE identification of a band comprising of the correct size and 4-methylumbelliferone (4-MU) caprylate hydrolysis activity. Pooled fractions were concentrated and buffer exchanged into 25 mM HEPES, 50 mM KCl, 2 mM CaCl₂, 5% glycerol, pH 7.0.

For purified lipase samples, microvolume protein concentration determination, in mg/mL, was obtained by using a Thermo Scientific NanoDrop 2000 Spectrophotometer. Specifically, a blank measurement was established by using a 2 μL aliquot of storage buffer (25 mM HEPES, 50 mM KCl, 2 mM CaCl₂), 5% glycerol, pH 7.0). Then, an aliquot of 2 μL lipase sample was used to measure the absorbance at 280 nm. A theoretical molar extinction coefficient (s=21,170 L·mol-1·cm-1) and a molecular weight of 31,280 Da were used to convert the Absorbance to mg/mL of protein by the NanoDrop Software. The measurement was repeated three to five times and the average value was reported.

The lipase concentration in the supernatant of shake flask culture samples was estimated by Ultra High Performance Liquid Chromatography, UHPLC, (Shimadzu Nexera X2 Series equipped with CBM-20A network-compatible controller, a LC-30AD solvent delivery unit, a SIL-30ACMP auto-sampler, a CTO-20AC column oven, and a SPD-M30A UV/Vis detector). Calibration curve, in the range of 125 to 2500 ppm, was generated by diluting purified standard lipase of SEQ ID NO: 1, in either 25 mM HEPES, pH 7.0 or in GRANT'S II media supplemented with 2 mM CaCl₂). Unknown samples were diluted (normally from flask experiment, one- to four-fold) in either 25 mM HEPES, pH 7.0 or in GRANT'S II media supplemented with 2 mM CaCl₂. All samples, standards and unknowns, were further diluted (five-fold) in molecular biology grade water. Blank samples, for subtracting the background absorbance, were prepared in water by five-fold dilution of either 25 mM HEPES, pH 7.0 or GRANT'S II, before filtration through a MultiScreenHTS HV Filter Plate (membrane of pore size 0.45 μm). Samples were eluted from the column (Agilent ZORBAX StableBond 300SB-C8 300 Å 1.8 μm, 2.1×50 mm (RRHD 1200 bar)) that was equilibrated at 40° C. using a gradient of 0.1% trifluoroacetic acid (TFA) in water and 0.1% TFA in acetonitrile. Absorbance was measured at 220 nm and peaks were integrated using the LabSolutions software (Shimadzu).

### Example 2

### General Methods

The following assays are standard assays used in the examples described below. Occasionally specific protocols call for deviations from these standard assays. In those cases, deviations from these standard assay protocols below are identified in the examples.

A. Wash Performance Index. PI(wash)

The wash performance index, PI(wash), of an enzyme compares the performance of the variant with a parent or reference polypeptide at the same protein concentration.

A PI(wash) that is greater than 1 (PI>1) indicates improved wash performance by a variant as compared to the parent or reference polypeptide (e.g., SEQ ID NO:1), while a PI of 1 (PI=1) identifies a variant that performs the same in washing as the parent or reference polypeptide, and a PI that is less than 1 (PI<1) identifies a variant that does not perform as well in wash performance as the parent or reference polypeptide. For example, the mature protein set forth as SEQ ID NO:1 is the parent of the T4K_L203R lipase variant.

The wash performance index (PI(wash)) of a lipase relative to a reference lipase was calculated as:

PI(wash)=((% SRI(variant)−% SRI(no enzyme))/((% SRI(lipase ref.)−% SRI(no enzyme)).

### B. Malodor Performance Index

A malodor performance index (PI(malodor)) that is less than 1 (PI(malodor)<1) indicates improved performance by a variant as compared to the reference polypeptide, while a PI(malodor) of 1 (PI=1) identifies a variant that performs the same as the reference polypeptide, and a PI(malodor) that is greater than 1 (PI>1) identifies a variant that does not perform as well as the reference polypeptide.

The malodor performance index (PI(malodor)) of a lipase is the ratio between the amount butanoic acid or hexanoic acid released (peak area) from a lipase-washed swatch and the amount butanoic acid or hexanoic acid released (peak area) from a reference lipase washed swatch, after both values have been corrected for the amount of butanoic acid or hexanoic acid released (peak area) from a non-lipase washed swatch (blank). The reference lipase is Lipex 100L. The malodor performance index (PI(malodor)) of the polypeptide is calculated as:

PI(malodor)=((malodor(variant)−malodor(no enzyme))/((malodor(lipase ref.)−malodor(no enzyme)),

where malodor is the butanoic acid or hexanoic acid (peak area) released from the textile surface measured using headspace gas chromatography/mass spectrometry (GC/MS) analysis.

### B. Hydrolysis of 4-Methylumbelliferone Caprylate Assay

The Lipase variants can be assayed for lipase activity on a 4-Methylumbelliferone (4-MU) caprylate substrate (octanoate) (Chem-Impex International, CAS20671-66-3). A reaction emulsion with 4-MU caprylate is prepared using 1 mM 4-MU caprylate pre-suspended in ethanol (100%) in 0.2% Triton X-100, 10 mM HEPES (pH 8.0), 250 ppm 3:1 Ca:Mg hardness.

The 4-MU caprylate/buffer suspensions are mixed and transferred to a 96-well microtiter plate (MTP) containing the enzyme sample, in a total volume of 100 μL. Dilution of the enzyme samples and their transfer volumes are adjusted to keep the reaction within a linear range. The release of 4-methylumbelliferone due to hydrolysis was monitored by measuring the fluorescence with the excitation at 380 nm and the emission at 450 nm for 5 minutes in 20 second intervals with a SpectraMax (Molecular Devices) corrected using blank values (no enzyme). The enzyme hydrolysis rate is calculated by dividing the fluorescence units measured over time.

### C. Wash Performance Assays

Miniswatch Assay with Ballast

The miniswatch assay was performed as follows. Detergent solutions were prepared by weighing out the appropriate mass of heat-treated detergent (Heat-inactivated commercial heavy-duty liquid laundry (HDL) laundry detergent, heated for 8 h at 80° C.). A volume of 3:1 Ca²⁺:Mg²⁺ Hardness stock solution was added to the detergent so that the final working solution contained 250 ppm of 3:1 Ca²⁺:Mg²⁺, then water was added to achieve 98% the final working volume. Enzyme solutions were prepared as a 50× solution in 500 mM HEPES pH 8.0. When the 50× enzyme solution was added to the 98% detergent solution the final working cleaning solution contained 1× enzyme, 10 mM HEPES pH 8.0, 250 ppm hardness and a manufacturer-recommended concentration of detergent. CS-61 swatches, which are pre-stained cotton swatches stained with beef fat and a red dye (Center for Testmaterials, CFT, The Netherlands) were used in a 24-well plate format. CN-42 swatches, which are unstained cotton knit fabric swatches, were also utilized as ballast. Swatches were prepared by cutting 12-mm square swatches from larger stained swatch material obtained from either TestFabrics Inc. or Center for Testmaterials. One square of CS-61 and ten squares of CN-42 were added to each well of a 24-well plate. 4.9 mL of the 98% detergent dilution was added to each well, and 100 μL of the 50× enzyme dilution was added to each well containing detergent. Plates were sealed with foil and incubated in a shaker incubator at 400 rpm for 30 min at the desired temperature. Shaking conditions were optimized using an Infors shaker with a 3-mm throw. After the incubation period, swatches were removed from the wells using tweezers and rinsed under cold running tap water to remove excess detergent and enzyme. Rinsed swatches were then placed on an absorbent paper towel and allowed to dry completely while protected from light. Color measurements of the dried swatches were performed using a hand-held reflectometer. Each individual piece of washed swatch material was placed on a black paper sheet and interrogated with the reflectometer. L*a*b* values were recorded and % Soil Removal Index (% SRI) was calculated using unwashed technical stain for before wash, washed technical stain for after wash and a white reflectometer calibration target as a reference measure of white.

CIE L*a*b* measurements of technical stains were made using a Minolta CR-400 Reflectometer with 8 mm aperture. % Soil Removal Index (% SRI) was calculated using the following equation:

$$\% S R I = \frac{Δ E ( After - Before Wash )}{Δ E ( White - Before Wash )} - \frac{\sqrt{{( {L}_{f} - {L}_{i} )}^{2} + {( {a}_{f} - {a}_{i} )}^{2} + {( {b}_{f} - {b}_{i} )}^{2}}}{\sqrt{{( {L}_{w} - {L}_{i} )}^{2} + {( {a}_{w} - {a}_{i} )}^{2} + {( {b}_{w} - {b}_{i} )}^{2}}} \times 100$$

where L_{f} is L* for after wash, Lᵢ is L* for before wash, and L_{w} is L* for white, where a_{f} is a* for after wash, aᵢ is a* for before wash, and a_{w} is a* for white, and where b_{f} is b* for after wash, bᵢ is b* for before wash, and b_{W} is b* for white, and where ΔE is defined as color difference as defined by L*a*b*.
Miniswatch Assay without Ballast

The miniswatch assay without ballast was performed as follows. Detergent solutions were prepared by weighing out the appropriate mass of heat-treated detergent (Heat-inactivated commercial HDL laundry detergent, 8 h at 80° C.). A volume 3:1 Ca²⁺:Mg²⁺ Hardness stock solution was added to the detergent so that the final working solution contained 250 ppm of 3:1 Ca²⁺:Mg²⁺, then water was added to achieve 95% the final working volume. Enzyme solutions were prepared as a 20× solution in 200 mM HEPES pH 8.0. When the 20× Enzyme solution was added to the 95% detergent solution the final working cleaning solution contained 1× enzyme, 10 mM HEPES pH 8.0, 250 ppm hardness and a manufacturer-recommended concentration of detergent. CS-61 swatches, which are pre-stained cotton swatches stained with beef fat and a red dye (Center for Testmaterial, CFT, The Netherlands) were used in a 24-well plate format. Swatches were prepared by cutting 12-mm square swatches from larger stained swatch material obtained from either TestFabricsInc. or Center for Testmaterial (Center for Testmaterial, CFT, The Netherlands). One square of CS-61 and 1.9 mL of the 95% detergent dilution was added to each well, and 100 μL of the 20× enzyme dilution was added to each well containing detergent. Plates were sealed with foil and incubated in a shaker incubator at 400 rpm for 30 min at the desired temperature. Shaking conditions were optimized using an Infors shaker with a 3-mm throw. After the incubation period, swatches were removed from the wells using tweezers and rinsed under cold running tap water to remove excess detergent and enzyme. Rinsed swatches were then placed on an absorbent paper towel and allowed to dry completely protected from light. Color measurements of the dried swatches were performed using a hand-held reflectometer. Each individual piece of washed swatch material was placed on a black paper sheet and interrogated with the reflectometer. L*a*b* values were recorded and % Soil Removal Index (% SRI) was calculated using unwashed technical stain for before wash, washed technical stain for after wash and a white reflectometer calibration target as a reference measure of white.

CIE L*a*b* measurements of technical stains were made using a Minolta CR-400 Reflectometer with 8 mm aperture. % Soil Removal Index (% SRI) was calculated using the following equation:

$$\% S R I = \frac{Δ E ( After - Before Wash )}{Δ E ( White - Before Wash )} - \frac{\sqrt{{( {L}_{f} - {L}_{i} )}^{2} + {( {a}_{f} - {a}_{i} )}^{2} + {( {b}_{f} - {b}_{i} )}^{2}}}{\sqrt{{( {L}_{w} - {L}_{i} )}^{2} + {( {a}_{w} - {a}_{i} )}^{2} + {( {b}_{w} - {b}_{i} )}^{2}}} \times 100$$

where L_{f} is L* for after wash, Lᵢ is L* for before wash, and L_{w} is L* for white, where a_{f} is a* for after wash, aᵢ is a* for before wash, and a_{w} is a* for white, and where b_{f} is b* for after wash, bᵢ is b* for before wash, and b_{w} is b* for white, and where ΔE is defined as color difference as defined by L*a*b*.

### Launderometer Assay

The launderometer (SDL Atlas, Model M228AA) was set to 40° C., or adjusted to 16° C. by adding ice to the launderometer water bath. For testing at 16° C., the wash solutions were first chilled to 13-16° C., then 200 mL of wash solution and enzymes were added to each launderometer test beaker and the resulting solution mixed well. Technical stains (8×12 cm) and unstained fabric swatches (8×12 cm) added as ballast were then added to the test beakers, which were then sealed and placed into the launderometer and the run initiated. At the end of the wash cycle, the swatches were removed from the test beakers and transferred to a 5-L plastic beaker, rinsed under running cold tap water for 1 minute, the rinse decanted, and the 1-min rinse cycle repeated three additional times. The swatches were dried in a spin dryer with no heat for 5 minutes, then dried in air at room temperature. L*a*b values were recorded and % Soil Removal Index (% SRI) was calculated using unwashed technical stain for before wash, washed technical stain for after wash and a white reflectometer calibration target as a reference measure of white

CIE L*a*b* measurements of technical stains were made using a Minolta CR-410 Reflectometer with 50 mm aperture. % Soil Removal Index (% SRI) was calculated using the following equation:

$$\% S R I = \frac{Δ E ( After - Before Wash )}{Δ E ( White - Before Wash )} - \frac{\sqrt{{( {L}_{f} - {L}_{i} )}^{2} + {( {a}_{f} - {a}_{i} )}^{2} + {( {b}_{f} - {b}_{i} )}^{2}}}{\sqrt{{( {L}_{w} - {L}_{i} )}^{2} + {( {a}_{w} - {a}_{i} )}^{2} + {( {b}_{w} - {b}_{i} )}^{2}}} \times 100$$

where L_{f} is L* for after wash, Lᵢ is L* for before wash, and L_{w} is L* for white, where a_{f} is a* for after wash, aᵢ is a* for before wash, and a_{w} is a* for white, and where b_{f} is b* for after wash, bᵢ is b* for before wash, and b_{w} is b* for white, and where ΔE is defined as color difference as defined by L*a*b*.

Miniswatch and Launderometer assays used commercial laundry detergents. For North American (NA) and Western European (WE) heavy duty liquid laundry (HDL) detergents, heat inactivation of the enzymes present in commercially-available detergents was performed by placing pre-weighed liquid detergent (in a sealed glass bottle) in a water bath at 80° C. for 8 hours. For testing of enzyme activity in heat-inactivated detergents, wash solutions of detergents were made from the heat inactivated HDL detergent stocks. Appropriate amounts of water hardness (e.g., 14.6 gpg or 17.5 gpg) and buffer were added to the wash solutions to match the desired conditions. The wash solutions were mixed by stirring prior to use.

In one aspect, a Persil Color Gel detergent (HDL detergent, WE region) was used wherein the laundry washing conditions were at a dose of 8.0 g/L detergent, 5 mM HEPES buffer (pH 8.0), 17.5 gpg of 3:1 Ca²⁺:Mg²⁺, and temperature at 16° C. or 40° C.

### D. Malodor Performance Assays

### Headspace Gas Chromatography/Mass Spectrometry (GC/MS) Analysis

Washed fabric swatch samples were sealed in 20-mL headspace glass vials (Agilent Technologies, part number 5182-0837) for instrumental analysis by headspace gas chromatography/mass spectrometry (GC/MS). All samples were stored refrigerated (ca. 4° C.) until analysis, or stored at room temperature for 10 days prior to analysis. Prior to headspace GC/MS analysis, all sealed vials containing washed fabric samples were placed in an oven and heated to 100° C. for one hour, taken out of the oven, and allowed to reach ambient temperature. The instrumentation used for the analysis consisted of an Agilent 7697A headspace sampler, coupled to an Agilent 7890A GC system, which was coupled to an Agilent 5975C single quadrupole mass spectrometer. Headspace sampler conditions were set as follows: oven temperature=105° C., injection loop temperature=125° C., transfer line temperature=145° C., vial equilibration time=120 min, injection duration 0.5 min. Gas chromatograph conditions were set as follows: inlet temperature=250° C., helium carrier gas flow=1.0 mL/min. A J&W Scientific DB-FFAP chromatographic column was used for the analysis. Column dimensions were 30 m length×0.250 mm inner diameter and 0.25-micrometer film. The column oven temperature program was as follows: initial column oven temperature of 60° C. which was held for 1 min, followed by a 20° C./min temperature ramp up to 240° C.; then, the temperature was held at 240° C. for 7 min. Mass spectrometer conditions were set as follows: m/z scan range=15-250, m/z scan rate=5.76 scans/second.

Under the conditions described above, the retention times of butanoic acid and hexanoic acid were approximately 8.3 min and 9.3 min, respectively. Peak area measurements were conducted using extracted ion chromatograms of m/z 60.

### E. Thermostability Assay and Calcium Ion Binding Stability Assay

Thermostability in HDL was assessed by heating lipase samples in a model HDL formulation at 45° C. for 30 minutes and comparing the activity measured following the stress treatment to an unstressed paired sample held on ice for the same incubation period. The model HDL comprised the following ingredients: 3.1% lauryl alcohol ethoxylate (6EO), 9% sodium lauryl ether sulphate, 3% sodium citrate, 0.8% sorbitol, 0.8% glycerol, 0.5% triethanolamine, 1% ethanol (absolute); 7.6% linear alkylbenzenesulfonate (LAS), 3.0% potassium cocoate, 2.5% propylene glycol, 0.01% 2-methyl-4-isothiazolin-3-one, 5.8% 4M NaOH and 62.9% demineralized water. Stability in HDL supplemented with EDTA (calcium ion binding stability) was separately used to assess stability in calcium limiting conditions. Calcium ion binding stability stability samples were prepared in the same manner as thermostability samples except 10 mM EDTA was added to the HDL formulation and the samples were incubated on ice for 30 minutes. After stress incubations, all samples were diluted 10-fold in 10 mM HEPES pH 8.0. Diluted samples were mixed by pipetting and a 20 μL volume was transferred to an opaque assay plate. A substrate solution was freshly prepared immediately prior to enzyme activity measurements that contained 2 mM 4-methylumbelliferyl-caprylate, 10 mM HEPES pH8, 100 mM NaCl, 1 mM CaCl₂, 0.2% Triton X100, and 1% bovine serum albumin. An 80 μL volume of the substrate solution was added to the samples and activity was measured by monitoring the fluorescent emission of liberated methylumbelliferone. A fluorescent plate reader (Spectromax M5, Molecular Devices) was set to an excitation wavelength of 380 nm and an emission wavelength of 450 nm and fluorescent emission levels were recorded at 20 second intervals over a three-minute period and raw data was recorded as fluorescent units/second. Background hydrolysis rates were measured in a no-enzyme control well containing buffer components but no added lipase and was used to subtract background hydrolysis from the sample wells. The baseline rate recorded from samples held at 4° C. was used to calculate the % residual activity of the paired stressed samples incubated at 45° C. or in the presence of EDTA. All measurements where stability is expressed as a performance index (PI) use the ratio of % residual activity measurements of variant samples over the % residual activity SEQ ID NO:1 parental strain. Samples with PI values >1 have more residual activity following thermal-stress or calcium ion binding stability (EDTA stress) in HDL compared the parent backbone.

The ratio of stressed vs. unstressed values was used to calculate the % residual activity and compared to the % residual activity of the parent lipase (SEQ ID NO:1). The relative increase in thermostability is expressed as a performance index (PI) where PI (thermostability)=% residual activity variant/% residual activity of parent lipase of SEQ ID NO:1.

### Example 3

### Wash Performance of Lipase Variants.

In order assess the cleaning performance of lipase variants of the parent lipase of SEQ ID NO:1 disclosed herein, the wash performance was determined using a miniswatch assay as well as using laundry washing experiments as described herein, where a wash performance, PI(wash) was determined

The wash performance of the lipase variants at 0.16 ppm was determined based on the % SRI (cleaning) against CS-61 (Beef fat on cotton) in the presence of ballast (CN-42) using the miniswatch assay with ballast described herein (Example 2) at 16° C. in Persil Color Gel using CS-61, 250 ppm 3:1 Ca²⁺:Mg²⁺ hardness, 30 min, 10 mM HEPES, pH 8.0 (Table 20). The wash performance index (PI(wash)) of a lipase variant relative to a reference lipase (SEQ ID NO:2) was calculated as:

$$P I ( Wash ) = ( ( \% S R I ( lipase variant ) - \% S R I ( no enzyme ) ) / ( ( \% S R I ( lipase reference ) - \% S R I ( no enzyme ) ) .$$

Lipase variants are referred to by their positional modification relative to the lipase of SEQ ID NO:1. For example, lipase variant “N146H_N280K” is a variant of SEQ ID NO: 1 that comprises a substitution at position 146 (N changed to H in variant) and a substitution at position 280 changed to K in variant).

TABLE 20
Wash Performance Index of lipase variants
of SEQ ID NO: 1 at 0.16 ppm

| | | Wash |
|---|---|---|
| | | perfor- |
| | | mance |
| Lipase variant (based on positions modified | | index, |
| relative to parent of SEQ ID NO: 1) | % SRI | PI(wash) |
| Reference (Parent lipase of SEQ ID NO: 1) | 20.7 ± 2.3 | 1.00 |
| T4K_N146H_I179K_L203R_L212R_N280K | 38.6 ± 0.6 | 5.48 |
| N146H_N280K | 37.9 ± 1.2 | 5.30 |
| T4K_N146H_A178K_L203R_G268K_N280K | 35.3 ± 3.2 | 4.65 |
| T4K_L212R | 35.2 ± 1.9 | 4.63 |
| N146H_A178K_I179K_G268K | 34.0 ± 0.8 | 4.33 |
| N146H_I179K | 33.9 ± 1.0 | 4.30 |
| A178K_I179K_L203R_L212R | 33.0 ± 1.4 | 4.08 |
| T4K_L203R_L212R_G268K | 32.5 ± 2.2 | 3.95 |
| I179K_L212R | 32.2 ± 2.3 | 3.88 |
| A178K_G268K | 30.0 ± 4.0 | 3.33 |
| N146H_A178K_I179K_L212R | 28.2 ± 1.2 | 2.88 |
| T4K_L203R | 24.7 ± 0.4 | 2.00 |
| No lipase | 16.7 + 1.7 | |

Table 20 shows the %/SRI and wash performance index of lipase variants. The % SRI in the absence of enzyme (%/SRI (no enzyme) was 16.7±1.7. A lipase variant is considered to exhibit improved wash performance if it performs better than the reference lipase, that is if the PI (wash) is greater than 1, PI(wash)>1.

Wash performance of lipase variants of lipase parent of SEQ ID NO:1 was also determined for oily soil removal using the launderometer assay (Example 2) and 0.64 ppm lipase at low temperature of 16° C. (Table 21). Reported % SRI was calculated from average of % SRI of two 8-cm×12-cm swatches (two replicate measurements of L*a*b* for each of two swatches) of CFT C-S-61, beef fat on cotton, per condition. Added ballast ranged from zero to six 8-cm×12-cm STC CFT CN-42 knitted cotton swatches. The wash performance index (PI(wash)) of a lipase variant relative to the reference lipase of SEQ ID NO:1 was calculated as:

$$P I ( Wash ) = ( ( \% S R I ( lipase variant ) - \% S R I ( no enzyme ) ) / ( ( \% S R I ( lipase reference ) - \% S R I ( no enzyme ) ) .$$

The wash performance was determined without any ballast(# of CN-42swatches=0) and with a ballast of 2,4,6 CN-42 swatches

TABLE 21
Wash performance of lipase variants of SEQ ID NO: 1 with and without ballast.
% SRI of CFT C-S-61, beef fat on cotton, in Persil Color Gel detergent at 16°
C., 300 ppm 3:1 Ca²⁺:Mg²⁺, 30 min, 5 mM HEPES (pH 8.0).

| CN-42 | | % SRI reference | % SRI | % SRI |
|---|---|---|---|---|
| ballast | no lipase | (SEQ ID NO: 1) | T4K_L203R | T4K_N146H_A178K_L203R_G268K_N280K |
| 0 | 36.8 ± 1.1 | 45.9 ± 0.3 | 46.9 ± 0.0 | 46.7 ± 0.9 |
| 2 | 35.8 ± 2.2 | 41.4 ± 0.1 | 45.7 ± 1.2 | 44.9 ± 1.2 |
| 4 | 26.0 ± 3.6 | 30.6 ± 1.3 | 38.6 ± 1.5 | 40.5 ± 0.2 |
| 6 | 18.6 ± 1.0 | 28.2 ± 0.4 | 37.7 ± 1.9 | 30.2 ± 1.7 |

| CN-42 | PI(wash) | PI(wash) |
|---|---|---|
| ballast | T4K_L203R | T4K_N145H_A178K_L203R_G268K_N280K |
| 0 | 1.11 | 1.09 |
| 2 | 1.77 | 1.63 |
| 4 | 2.74 | 3.15 |
| 6 | 1.99 | 1.21 |

Wash performance of lipase variants of lipase parent of SEQ ID NO:1 was also determined for oily soil removal using the launderometer assay (Example 2) and 0.64 ppm lipase at 40% C (Table 22). Reported % SRI was calculated from average of % SRI of two 8-cm×12-cm swatches (two replicate measurements of L*a*b* for each of two swatches) of CFT C-S-61, beef fat on cotton, per condition. Added ballast ranged from zero to six 8-cm×12-cm STC CFT CN-42 knitted cotton swatches. The wash performance was determined without any ballast (# of CN-42 swatches=0) and with a ballast of 2, 4, or 6 CN-42 swatches.

TABLE 22
Wash performance of lipase variants of SEQ ID NO: 1 with and without
ballast. % SRI of CFT C-S-61, beef fat on cotton, in Persil Color Gel at 40°
C., 300 ppm 3:1 Ca²⁺:Mg²⁺, 30 min, 5 mM HEPES (pH 8.0).

| CN-42 | | % SRI reference | % SRI | % SRI |
|---|---|---|---|---|
| ballast | no lipase | (SEQ ID NO: 1) | T4K_L203R | T4K_N146H_A178K_L203R_G268K_N280K |
| 0 | 60.6 ± 0.6 | 61.9 ± 0.4 | 61.9 ± 0.4 | 62.2 ± 0.4 |
| 2 | 61.6 ± 0.7 | 61.2 ± 0.7 | 62.8 ± 0.1 | 62.5 ± 0.4 |
| 4 | 58.3 ± 0.3 | 62.6 ± 0.7 | 61.1 ± 0.1 | 62.7 ± 0.6 |
| 6 | 51.8 ± 2.2 | 63.2 ± 0.1 | 62.2 ± 0.7 | 63.3 ± 0.6 |

| CN-42 | PI(wash) | PI(wash) |
|---|---|---|
| ballast | T4K_L203R | T4K_N146H_A178K_L203R_G268K_N280K |
| 0 | 1.00 | 1.23 |
| 2 | nd | nd |
| 4 | 0.65 | 1.02 |
| 6 | 0.91 | 1.01 |

Nd = not determined as % SRI of lipase is smaller than % SRI of no lipase (equivalent % SRI with or without added lipase within standard deviation of measurement at 40° C.).

Wash performance of lipase variants of lipase parent of SEQ ID NO:1 was also determined for oily soil removal using the launderometer assay (Example 2) and different lipase concentrations, from 0 ppm to 0.64 ppm lipase at 40° C. (Table 23A and 23B). Reported % SRI was calculated from average of % SRI of two 8-cm×12-cm swatches (two replicate measurements of L*a*b* for each of two swatches) of CFT C-S-61, beef fat on cotton, per condition. Added ballast was six 8-cm×12-cm STC CFT CN-42 knitted cotton swatches.

TABLE 23A
% SRI of lipase variants at different lipase concentrations. % SRI for
SEQ ID NO: 1 variants using CFT C-S-61, beef fat on cotton, in Persil Color Gel at 40°
C., 300 ppm 3:1 Ca²⁺:Mg²⁺, 30 min, 5 mM HEPES (pH 8.0).

| | | | | % SRI | |
|---|---|---|---|---|---|
| | | | | T4K_N146H_{—} | % SRI |
| | % SRI | | % SRI | I179K_{—} | T4K_N146H |
| | reference | % SRI | N146H_A178 | L203R_{—} | _A178K_{—} |
| lipase | (SEQ ID | T4K_L203 | K_I179K_{—} | L212R_{—} | L203R_G268 |
| (ppm) | NO: 1) | R | G268K | N280K | K_N280K |
| 0 | 57.9 ± 1.4 | 57.9 ± 1.4 | 57.9 ± 1.4 | 57.9 ± 1.4 | 57.9 ± 1.4 |
| 0.16 | 62.8 ± 0.5 | 62.7 ± 1.1 | 62.4 ± 0.3 | 62.3 ± 0.5 | 62.6 ± 1.5 |
| 0.32 | 63.1 ± 0.1 | 63.0 ± 0.6 | 63.2 ± 0.5 | 63.0 ± 0.9 | 64.1 ± 0.4 |
| 0.64 | 63.8 ± 1.3 | 63.4 ± 1.4 | 64.2 ± 1.1 | 61.7 ± 0.9 | 61.9 ± 0.9 |

TABLE 23B
Wash performance (PI(wash) of lipase variants at different lipase concentrations
using CFT C-S-61, beef fat on cotton, in Persil Color Gel at 40° C., 300 ppm
3:1 Ca²⁺:Mg²⁺, 30 min, 5 mM HEPES (pH 8.0).

| | | | | PI(wash) | |
|---|---|---|---|---|---|
| | | | | T4K_N146H_{—} | PI(wash) |
| | PI(wash) | | PI(wash) | I179K_{—} | T4K_N146H |
| | reference | PI(wash) | N146H_A178 | L203R_{—} | _A178K_{—} |
| lipase | (SEQ ID | T4K_L203 | K_I179K_{—} | L212R_{—} | L203R_G268 |
| (ppm) | NO: 1) | R | G268K | N280K | K_N280K |
| 0.16 | 1.00 | 0.98 | 0.92 | 0.90 | 0.96 |
| 0.32 | 1.00 | 0.98 | 1.02 | 0.98 | 1.19 |
| 0.64 | 1.00 | 0.93 | 1.07 | 0.64 | 0.68 |

Wash performance of lipase variants of lipase parent of SEQ ID NO:1 was also determined for oily soil removal using the launderometer assay (Example 2) and 0.64 ppm lipase at 16° C. using beef fat on polyester (Table 24). % SRI calculated from average %/SRI from two 8-cm×12-cm swatches (two replicate measurements of L*a*b* for each of two swatches) of CFT P-S-61, beef fat on polyester, per condition. Added ballast ranged from zero to twelve 8-cm×12-cm swatches of STC CFT PN-01 unsoiled polyester swatches.

TABLE 24
Wash performance of lipase variants of SEQ ID NO: 1. % SRI of CFT P-S-61, beef fat on polyester,
in Persil Color Gel at 16° C., 300 ppm 3:1 Ca²⁺:Mg²⁺ 30 min, 5 mM HEPES (pH 8.0).

| PN-01 | | | | |
|---|---|---|---|---|
| ballast (# | | Parent Lipase | | |
| swatches) | no lipase | SEQ ID NO: 1 | T4K_L203R | T4K_N146H_A178K_L203R_G268K_N280K |
| 0 | 41.7 ± 0.8 | 43.1 ± 0.6 | 37.3 ± 0.6 | 41.5 ± 0.2 |
| 4 | 28.8 ± 0.7 | 40.5 ± 2.4 | 42.1 ± 1.3 | 34.9 ± 2.5 |
| 8 | 25.4 ± 0.2 | 39.2 ± 1.8 | 38.9 ± 0.7 | 38.9 ± 1.9 |
| 12 | 18.0 ± 1.1 | 35.1 ± 0.3 | 38.7 ± 2.7 | 35.8 ± 3.1 |

| PN-01 | PI(wash) | | |
|---|---|---|---|
| ballast (# | Parent Lipase | PI(wash) | PI(wash) |
| swatches) | SEQ ID NO: 1 | T4K_L203R | T4K_N146H_A178K_L203R_G268K_N280K |
| 0 | — | — | — |
| 4 | 1.00 | 1.14 | 0.52 |
| 8 | 1.00 | 0.98 | 0.98 |
| 12 | 1.00 | 1.21 | 1.04 |

Wash performance of lipase variants of lipase parent of SEQ ID NO:1 was also determined for oily soil removal using the launderonmeter assay (Example 2) and 0 ppm to 0.64 ppm lipase at 16° C. Reported % SRI was calculated from average of % SRI of two 8-cm×12-cm swatches (two replicate measurements of L*a*b* for each of two swatches) of CFT C-S-61, beef fat on cotton, per condition. Added ballast ranged from zero to six 8-cm×12-cm STC CFT CN-42 knitted cotton swatches (Table 25A and 25B).

TABLE 25A
% SRI for Lipase variants of parent lipase of SEQ ID NO: 1, using CFT C-S-61, beef fat on
cotton, in Persil Color Gel at 16° C., 300 ppm 3:1 Ca²⁺:Mg²⁺, 30 min, 5 mM HEPES (pH 8.0).

| lipase | % SRI | % SRI | % SRI | % SRI | % SRI | % SRI | % SRI |
|---|---|---|---|---|---|---|---|
| (ppm) | SEQ ID NO: 1 | A159D | G206E_G209D | G206E_G209A | N146H_I179K | T4K_L212R | A178K_I179K_L203R_L212R |
| 0 | 22.2 ± 2.8 | 22.2 ± 2.8 | 22.2 ± 2.8 | 22.2 ± 2.8 | 22.2 ± 2.8 | 22.2 ± 2.8 | 22.2 ± 2.8 |
| 0.32 | 32.8 ± 0.4 | 30.8 ± 1.3 | 25.6 ± 1.6 | 29.0 ± 2.7 | 31.5 ± 0.4 | 30.8 ± 1.7 | 34.9 ± 0.5 |
| 0.64 | 34.4 ± 0.1 | 35.0 ± 0.9 | 30.4 ± 2.0 | 35.0 ± 0.1 | 33.8 ± 0.4 | 34.6 ± 0.9 | 38.9 ± 1.7 |

TABLE 25B
Wash performance of lipase variants of SEQ ID NO: 1. PI(wash) for lipase variants using CFT C-S-61, beef
fat on cotton, in Persil Color Gel at 16° C., 300 ppm 3:1 Ca²⁺:Mg²⁺, 30 min, 5 mM HEPES (pH 8.0).

| lipase | PI(wash) | PI(wash) | PI(wash) | PI(wash) | PI(wash) | PI(wash) |
|---|---|---|---|---|---|---|
| (ppm) | A159D | G206E_G209D | G206E_G209A | N146H_I179K | T4K_L212R | A178K_I179K_L203R_L212R |
| 0.32 | 0.809 | 0.323 | 0.641 | 0.874 | 0.808 | 1.190 |
| 0.64 | 1.048 | 0.672 | 1.053 | 0.955 | 1.018 | 1.367 |

### Example 4

### Detergent Stability of Lipase Variants

Crude Bacilus subtilis supernatant containing lipase was ultra-concentrated using 3 kDa molecular weight cut off filtration (Cat. No. UFC9003 EMD Millipore). The concentrated material was formulated in 10 mM HEPES (pH 8.0), 250 ppm 3:1 Ca²⁺:Mg²⁺ hardness, and 16% glycerol. A 1% (v/v) of the formulated material was added to full strength HDL Persil Color Gel detergent. The lipase activity was assessed in timepoints over a period of 96 hours using 4-methylumbelliferone caprylate as a substrate. The lipase samples were each diluted with an emulsion to a final concentration of 1 mM 4-methylumbelliferone caprylate, 0.2% Triton X-100, 10 mM HEPES (pH 8.0), 250 ppm 3:1 Ca²⁺:Mg²⁺ hardness. Samples were added to a Corning* 96 Well Solid Polystyrene Microplate (Sigma Cat. No. CLS3915). The release of 4-methylumbelliferone due to hydrolysis was monitored by measuring the fluorescence with the excitation at 380 nm and the emission at 450 nm for 5 minutes in 20 second intervals with a SpectraMax (Molecular Devices). Percent Lipase activity remaining in HDL Stability study after a certain time point (24 hrs, 48 hrs, 72 hrs and 96 hrs) are shown in Tables 26 and 27.

TABLE 26
Percent lipase activity remaining in HDL Stability study for lipase variants of parent lipase of SEQ ID NO: 1

| | % activity remaining | | |
|---|---|---|---|
| | Parent | | |
| | (SEQ ID | | |
| Time | NO: 1) | A29R_I64V_V70A_S95N_S227T_I272V | A29R_I64V_V70A_S95N_S227T_R267P_I272V |
| 24 hr | 41.1 | 89.1 | 0.3 |
| 48 hr | 17.8 | 50.1 | 2.4 |
| 72 hr | 12.5 | 14.5 | 2.0 |
| 96 hr | 20.2 | 14.9 | 2.8 |

| | % activity remaining | | | | |
|---|---|---|---|---|---|
| Time | I64V | V70A | S227T | I272V | R267P |
| 24 hr | 56.4 | 114.7 | 102.6 | 49.0 | 0.7 |
| 48 hr | 66.7 | 73.7 | 78.4 | 25.3 | 2.7 |
| 72 hr | 25.0 | 30.2 | 31.5 | 22.1 | 2.1 |
| 96 hr | 24.1 | 31.7 | 38.7 | 22.3 | 1.9 |

Table 26 shows that the Lipase variants A29R_I64V_V70A_S95N_S227T_I272V, I64V, V70A, S227T and I272V had an increased stability when compared to the reference lipase of SEQ ID NO:1.

TABLE 27
Percent lipase activity remaining in HDL Stability study for lipase variants of parent lipase of SEQ ID NO: 1

| | Parent of | | | | | | |
|---|---|---|---|---|---|---|---|
| | SEQ ID | | | | | | |
| | NO: 1 | A159D | G206E_G209D | G206E_G209A | N146H_I179K | N146H_N280K | T4K_L212R |
| 24 hr | 96.9 | 108.9 | 97.3 | 94.7 | 89.8 | 92.0 | 96.0 |
| 48 hr | 69.4 | 90.3 | 100.8 | 88.6 | 74.9 | 82.2 | 82.2 |
| 72 hr | 55.7 | 75.1 | 102.8 | 71.3 | 59.8 | 62.1 | 65.4 |
| 96 hr | 46.8 | 89.6 | 101.6 | 65.5 | 51.5 | 51.2 | 56.3 |

| | N146H_A178K_{—} | A178K_I179K_{—} | T4K_N146H_I179K_{—} | T4K_N146H_A178K_{—} |
|---|---|---|---|---|
| | I179K_G268K | L203R_L212R | L203R_L212R_N280K | I179K_L203R_G268K |
| 24 hr | 34.0 | 100.3 | 104.6 | 96.7 |
| 48 hr | 18.8 | 91.4 | 84.8 | 65.5 |
| 72 hr | 5.6 | 86.8 | 76.7 | 47.7 |
| 96 hr | 3.5 | 81.9 | 90.7 | 54.0 |

Table 27 shows that the Lipase variants A159D, G206E_G209D, G206E_G209A, N146H_I179K, N146H_N280K, T4K_L212R, A178K_I179K_L203R_L212R, T4K_N146H_I179K_L203R_L212R_N280K, T4K_N146H_A178K_I179K_L203R_G268K, had an increased stability when compared to the reference lipase of SEQ ID NO:1.

The cleaning activity (% SRI) of these variants was evaluated on beef fat on cotton (CS-61) in Persil Color Gel at 16° C., 250 ppm 3:1 Ca²⁺:Mg²⁺ hardness, 30 min, 10 mM HEPES, pH 8.0. % SRI indicated that all these variants had a comparable cleaning activity to the lipase reference of SEQ ID NO:1.

### Example 5

Lipase Variants with Improved Resistance to Proteolytic Degradation

The kinetics of proteolytic degradation of parent lipase of SEQ ID NO:1 and lipase variants L203P and A205P at 35° C. was monitored by SDS-PAGE analysis. Specifically, purified parent lipase and selected variants, listed in Table 28, at concentration of 3 mg/mL were incubated with Preferenz™ P300 protease (20:1 ratio lipase/protease) in 50 mM NH₄HCO₃, 2 mM CaCl₂), pH 7.6 at 35° C. with shaking (500 rpm) in an Eppendorf Thermomixer. In control samples, the protease was replaced with 25 mM HEPES, 2 mM CaCl₂, pH 7.0. At the time intervals specified in Table 28, 20 μL aliquots were removed and added to 80 μL ice-cold acetone containing 1 mM PMSF to stop the proteolysis. To precipitate the peptide fragments after proteolysis, the acetone samples were kept at −20° C. for 30 min before centrifugation at 4° C. for 5 min. Acetone was removed and the pellets were dried briefly under a stream of nitrogen. The pellets were re-dissolved in water, 0.1% TFA and one volume of sample was mixed with one volume of 2× Laemmli sample buffer (Bio-Rad) before loading onto a MINI-PROTEAN TGX 4-15% protein gel. After staining the gel for 1 h with Coomassie brilliant blue R-250 and destaining overnight, the peptide bands were visualized in a Gel Doc XR+ System (Bio-Rad). The progress of the proteolytic degradation of the lipase was determined by measuring the band intensities corresponding to native protein using densitometric scanning software Image Lab 6.0 (Bio-Rad) and normalized by setting the band intensity of the lipase in the control sample as 100% (Table 28).

TABLE 28
Kinetics of proteolytic degradation at 35° C. of parent lipase of
SEQ ID NO: 1 and lipase variants L203P and A205P derived
from SEQ ID NO: 1.
protein recovery (%)

| | Parent lipase | | |
|---|---|---|---|
| | reference of SEQ ID | Lipase variant | Lipase variant |
| Time (h) | NO: 1 | A205P | L203P |
| 0.0 | 100 | 100 | 100 |
| 0.5 | 30 | 92 | 80 |
| 1.0 | 28 | 73 | 54 |
| 2.0 | 21 | 73 | 57 |
| 4.5 | 13 | 54 | 41 |

Table 28 indicates that the lipase variants of the parent lipase SEQ ID NO:1 having a single amino acid substitution at position 203 (L203P) or position 205 (A205P) show an improved resistance to proteolytic degradation, also referred to as an improved protease stability. The lipolytic enzyme variants of A205P and L203P show an improved protease stability of at least 2-fold, 3-old, and at least 4-fold increased (for example 54/13=4.1 for A205P and 41/13=3.1 for L203P after 4.5 h) when compared to the parent lipase.

The kinetics of proteolytic degradation of SEQ ID NO:1 and lipase variants A205P and D122P_A205P at 40° C. was monitored by SDS-PAGE analysis, following the same procedure as describe above. The progress of the proteolytic degradation of the lipase was determined by measuring the band intensities corresponding to native protein using densitometric scanning software Image Lab 6.0 (Bio-Rad) and normalized by setting the band intensity of the lipase in the control sample as 100% (Table 29).

TABLE 29
Kinetics of proteolytic degradation at 40° C. of parent lipase of
SEQ ID NO: 1 and lipase variants L205P and D122P L205P derived
from SEQ ID NO: 1.
protein recovery (%)

| | Parent lipase | | |
|---|---|---|---|
| | reference of SEQ ID | Lipase variant | Lipase variant |
| Time (h) | NO: 1 | A205P | D122P_A205P |
| 0.0 | 100 | 100 | 100 |
| 0.5 | 20.7 | 90.8 | 79.3 |
| 1.0 | 19.9 | 74.7 | 73.6 |
| 3.9 | 10.5 | 37.5 | 55.3 |
| 5.0 | 7.8 | 30.2 | 51.6 |
| 6.5 | 7.6 | 27.2 | 42.3 |

Table 29 indicates that the lipase variant of the parent lipase SEQ ID NO:1 having a single amino acid substitution at position 205 (A205P) or positions 122 and 205 (D122P_A205P) show an improved resistance to proteolytic degradation, also referred to as an improved protease stability. The lipolytic enzyme variants of A205P and D122P_A205P show an improved protease stability of at least 3-fold, 4-fold, and at least 5-fold increased (for example 27.2/7.6=3.6 for A205P and 42.3/7.6=5.6 for D122P_A205P after 6.5 h) when compared to the parent lipase.

### Example 6

### Malodor Performance of Lipase Variants

Malodor performance of the lipase of SEQ ID NO:1 and lipase variants derived thereof were compared to a commercially available lipase enzyme of SEQ ID NO: 2 (also referred to as Lipex® L100). Malodor was determined via headspace gas chromatography/mass spectrometry (GC/MS) analysis as described in Example 2.

Malodor performance of lipase enzymes was analyzed for fabric swatches stained with butterfat on cotton (Tables 30-35) or chocolate ice cream on cotton (Table 36-37), where the swatches were washed using the launderometer assay (Example 2). Added ballast was six 8-cm×12-cm STC CFT CN-42 knitted cotton swatches. For each of the lipases tested, 2 CFT C-S-10, butterfat on cotton swatches or two CFT C-S-68, chocolate ice cream on cotton swatches (replicate A, replicate B) were tested at two different lipase concentrations (0.32 ppm and 0.64 ppm lipase).

The performance index for malodor was calculated as follows: PI(malodor)=(malodor(lipase variant)−malodor(no enzyme))/(malodor(lipase reference.)−malodor(no enzyme)), wherein the lipase reference was the commercially available lipase enzyme of SEQ ID NO: 2.

The malodor performance index was determined based on the production of butanoic acid or hexanoic acid. For example, PI(malodor) based on butanoic acid was calculated as follows: (butanoic peak area (lipase variant)−butanoic peak area (no lipase))/(butanoic peak area (reference lipase of SEQ ID NO: 7)−butanoic peak area (no lipase)).

TABLE 30
Malodor Performance Index using CFT C-S-10, butterfat on cotton, in Persil
Color Gel at 16° C., 300 ppm 3:1 Ca²⁺:Mg²⁺, 30 min, 5 mM HEPES (pH 8.0).

| | | | hexanoic | | hexanoic |
|---|---|---|---|---|---|
| | lipase | butanoic acid | acid (peak | butanoic acid | acid |
| Lipase (replicate A, B) | (ppm) | (peak area) | area) | PI(malodor) | PI(malodor) |
| Lipex 100L (A) | 0.32 | 39361 | 22378 | 1.000 | 1.000 |
| Lipex 100L (B) | 0.32 | 42280 | 25729 | 1.000 | 1.000 |
| Lipase parent (SEQ ID | 0.32 | 6787 | 6219 | 0.080 | 0.142 |
| NO: 1) (A) | | | | | |
| Lipase parent (SEQ ID | 0.32 | 6087 | 6877 | 0.053 | 0.131 |
| NO: 1) (B) | | | | | |
| N146H_ A178K_{—} | 0.32 | 8941 | 7945 | 0.141 | 0.233 |
| I179K_ G268K (A) | | | | | |
| N146H_ A178K_{—} | 0.32 | 12192 | 10988 | 0.212 | 0.321 |
| I179K_ G268K (B) | | | | | |
| T4K_ N146H_{—} | 0.32 | 6999 | 9838 | 0.086 | 0.334 |
| I179K_ L203R_{—} | | | | | |
| L212R_ N280K (A) | | | | | |
| T4K_ N146H_{—} | 0.32 | 6629 | 8224 | 0.067 | 0.193 |
| I179K_ L203R_{—} | | | | | |
| L212R_ N280K (B) | | | | | |
| no lipase (A) | 0.00 | 3956 | 3549 | — | — |
| no lipase (B) | 0.00 | 4078 | 4025 | — | — |

TABLE 31
Malodor Performance Index using CFT C-S-10, butterfat on cotton, in Persil
Color Gel at 16° C., 300 ppm 3:1 Ca²⁺:Mg²⁺, 30 min, 5 mM HEPES (pH 8.0).

| | | | hexanoic | | hexanoic |
|---|---|---|---|---|---|
| | lipase | butanoic acid | acid (peak | butanoic acid | acid |
| Lipase (replicate A, B) | (ppm) | (peak area) | area) | PI(malodor) | PI(malodor) |
| Lipex 100L (A) | 0.64 | 66821 | 38105 | 1.000 | 1.000 |
| Lipex 100L (B) | 0.64 | 63699 | 38010 | 1.000 | 1.000 |
| Lipase parent (SEQ ID | 0.64 | 5126 | 7032 | 0.019 | 0.101 |
| NO: 1) (A) | | | | | |
| Lipase parent (SEQ ID | 0.64 | 5004 | 7255 | 0.016 | 0.095 |
| NO: 1 (B) | | | | | |
| N146H_ A178K_{—} | 0.64 | 9986 | 11066 | 0.096 | 0.218 |
| I179K_ G268K (A) | | | | | |
| N146H_ A178K_{—} | 0.64 | 9418 | 10167 | 0.090 | 0.181 |
| I179K_ G268K (B) | | | | | |
| T4K_ N146H_ I179K_{—} | 0.64 | 7587 | 10771 | 0.058 | 0.209 |
| L203R_ L212R_{—} | | | | | |
| N280K (A) | | | | | |
| T4K_ N146H_ I179K_{—} | 0.64 | 7535 | 10099 | 0.058 | 0.179 |
| L203R_ L212R_{—} | | | | | |
| N280K (B) | | | | | |
| no lipase (A) | 0.00 | 3956 | 3549 | — | — |
| no lipase (B) | 0.00 | 4078 | 4025 | — | — |

TABLE 32
Malodor Performance Index using CFT C-S-10, butterfat on cotton, in Persil
Color Gel at 16° C., 300 ppm 3:1 Ca²⁺:Mg²⁺, 30 min, 5 mM HEPES (pH 8.0).

| | lipase | butanoic acid | hexanoic acid | butanoic acid | hexanoic acid |
|---|---|---|---|---|---|
| Lipase (replicate A, B) | (ppm) | (peak area) | (peak area) | PI(malodor) | PI(malodor) |
| Lipex 100L (A) | 0.32 | 164828 | 117649 | 1.000 | 1.000 |
| Lipex 100L (B) | 0.32 | 159811 | 110141 | 1.000 | 1.000 |
| Lipase parent (SEQ ID NO: 1) (A) | 0.32 | 30060 | 36836 | 0.085 | 0.183 |
| Lipase parent (SEQ ID NO: 1 (B) | 0.32 | 21131 | 32409 | 0.016 | 0.119 |
| T4K_L203R (A) | 0.32 | 23846 | 31971 | 0.043 | 0.134 |
| T4K_L203R (B) | 0.32 | 20937 | 29764 | 0.024 | 0.120 |
| N146H_N280K (A) | 0.32 | 22309 | 26643 | 0.032 | 0.080 |
| N146H_N280K (B) | 0.32 | 20394 | 28249 | 0.011 | 0.071 |
| T4K_N146H_A178K_L203R_G268K_N280K (A) | 0.32 | 26634 | 30832 | 0.062 | 0.122 |
| T4K_N146H_A178K_L203R_G268K_N280K (B) | 0.32 | 32941 | 36674 | 0.100 | 0.167 |
| A178K_G268K (A) | 0.32 | 24798 | 23181 | 0.049 | 0.045 |
| A178K_G268K (B) | 0.32 | 41459 | 51953 | 0.160 | 0.340 |
| no lipase (A) | 0.00 | 17523 | 18761 | — | — |
| no lipase (B) | 0.00 | 18860 | 21944 | — | — |

TABLE 33
Malodor Performance Index using CFT C-S-10, butterfat on cotton, in Persil
Color Gel at 16° C., 300 ppm 3:1 Ca²⁺:Mg²⁺, 30 min, 5 mM HEPES (pH 8.0).

| | lipase | butanoic acid | hexanoic acid | butanoic acid | hexanoic acid |
|---|---|---|---|---|---|
| Lipase (replicate A, B) | (ppm) | (peak area) | (peak area) | PI(malodor) | PI(malodor) |
| Lipex 100L (A) | 0.64 | 225888 | 141785 | 1.000 | 1.000 |
| Lipex 100L (B) | 0.64 | 266502 | 171100 | 1.000 | 1.000 |
| Lipase parent (SEQ ID NO: 1) (A) | 0.64 | 29194 | 37319 | 0.056 | 0.151 |
| Lipase parent (SEQ ID NO: 1 (B) | 0.64 | 28579 | 36719 | 0.039 | 0.099 |
| T4K_L203R (A) | 0.64 | 25224 | 29996 | 0.037 | 0.091 |
| T4K_L203R (B) | 0.64 | 27052 | 33444 | 0.033 | 0.077 |
| N146H_N280K (A) | 0.64 | 21795 | 30458 | 0.021 | 0.095 |
| N146H_N280K (B) | 0.64 | 26443 | 34894 | 0.031 | 0.087 |
| T4K_N146H_A178K_L203R_G268K_N280K (A) | 0.64 | 39113 | 45254 | 0.104 | 0.215 |
| T4K_N146H_A178K_L203R_G268K_N280K (B) | 0.64 | 35097 | 42412 | 0.066 | 0.137 |
| A178K_G268K (A) | 0.64 | 33723 | 35038 | 0.078 | 0.132 |
| A178K_G268K (B) | 0.64 | 33382 | 36297 | 0.059 | 0.096 |
| no lipase (A) | 0.00 | 17523 | 18761 | — | — |
| no lipase (B) | 0.00 | 18860 | 21944 | — | — |

TABLE 34
Malodor Performance Index using CFT C-S-10, butterfat on cotton, in Persil
Color Gel at 16° C., 300 ppm 3:1 Ca²⁺:Mg²⁺, 30 min, 5 mM HEPES (pH 8.0).

| | lipase | butanoic acid | hexanoic acid | butanoic acid | hexanoic acid |
|---|---|---|---|---|---|
| Lipase (replicate A, B) | (ppm) | (peak area) | (peak area) | PI(malodor) | PI(malodor) |
| Lipex 100L (A) | 0.32 | 102117 | 84406 | 1.000 | 1.000 |
| Lipex 100L (B) | 0.32 | 96401 | 85883 | 1.000 | 1.000 |
| Lipase parent (SEQ ID NO: 1) (A) | 0.32 | 20916 | 34909 | 0.171 | 0.359 |
| Lipase parent (SEQ ID NO: 1 (B) | 0.32 | 20132 | 35522 | 0.173 | 0.360 |
| A159D (A) | 0.32 | 18473 | 25082 | 0.146 | 0.232 |
| A159D (B) | 0.32 | 13710 | 24477 | 0.103 | 0.220 |
| G206E_G209D (A) | 0.32 | 13293 | 14968 | 0.093 | 0.101 |
| G206E_G209D (B) | 0.32 | 20622 | 22121 | 0.178 | 0.190 |
| G206E_G209A (A) | 0.32 | 12701 | 17970 | 0.087 | 0.140 |
| G206E_G209A (B) | 0.32 | 13925 | 21244 | 0.105 | 0.179 |
| N146H_I179K (A) | 0.32 | 13260 | 19783 | 0.093 | 0.163 |
| N146H_I179K (B) | 0.32 | 20708 | 30455 | 0.179 | 0.296 |
| T4K_L212R (A) | 0.32 | 15425 | 23294 | 0.115 | 0.209 |
| T4K_L212R (B) | 0.32 | 15215 | 23676 | 0.119 | 0.210 |
| A178K_I179K_L203R_L212R (A) | 0.32 | 10281 | 15504 | 0.062 | 0.108 |
| A178K_I179K_L203R_L212R (B) | 0.32 | 11633 | 19401 | 0.081 | 0.155 |
| K94D_N149D_A178D_K185D_G202E_N280E (A) | 0.32 | 13147 | 12102 | 0.091 | 0.064 |
| K94D_N149D_A178D_K185D_G202E_N280E (B) | 0.32 | 14944 | 15952 | 0.117 | 0.112 |
| no lipase (A) | 0.00 | 4200 | 7172 | — | — |
| no lipase (B) | 0.00 | 4247 | 7425 | — | — |

TABLE 35
Malodor Performance Index using CFT C-S-10, butterfat on cotton, in Persil
Color Gel at 16° C., 300 ppm 3:1 Ca²⁺:Mg²⁺ 30 min, 5 mM HEPES (pH 8.0).

| | lipase | butanoic acid | hexanoic acid | butanoic acid | hexanoic acid |
|---|---|---|---|---|---|
| Lipase (replicate A, B) | (ppm) | (peak area) | (peak area) | PI(malodor) | PI(malodor) |
| Lipex 100L (A) | 0.64 | 153615 | 113484 | 1.000 | 1.000 |
| Lipex 100L (B) | 0.64 | 158068 | 112014 | 1.000 | 1.000 |
| Lipase parent (SEQ ID NO: 1) (A) | 0.64 | 22314 | 35584 | 0.121 | 0.267 |
| Lipase parent (SEQ ID NO: 1 (B) | 0.64 | 19331 | 32756 | 0.098 | 0.244 |
| A159D (A) | 0.64 | 16013 | 24816 | 0.079 | 0.166 |
| A159D (B) | 0.64 | 17694 | 28214 | 0.088 | 0.201 |
| G206E_G209D (A) | 0.64 | 16538 | 22311 | 0.083 | 0.142 |
| G206E_G209D (B) | 0.64 | 13723 | 17067 | 0.062 | 0.094 |
| G206E_G209A (A) | 0.64 | 13914 | 21835 | 0.065 | 0.138 |
| G206E_G209A (B) | 0.64 | 14234 | 21915 | 0.065 | 0.141 |
| N146H_I179K (A) | 0.64 | 13679 | 20994 | 0.063 | 0.130 |
| N146H_I179K (B) | 0.64 | 14759 | 24215 | 0.069 | 0.163 |
| T4K_L212R (A) | 0.64 | 13812 | 20319 | 0.064 | 0.124 |
| T4K_L212R (B) | 0.64 | 13622 | 19109 | 0.061 | 0.114 |
| A178K_I179K_L203R_L212R (A) | 0.64 | 18169 | 32051 | 0.093 | 0.234 |
| A178K_I179K_L203R_L212R (B) | 0.64 | 16093 | 26296 | 0.077 | 0.182 |
| K94D_N149D_A178D_K185D_G202E_N280E (A) | 0.64 | 16241 | 13718 | 0.081 | 0.062 |
| K94D_N149D_A178D_K185D_G202E_N280E (B) | 0.64 | 17888 | 15816 | 0.089 | 0.082 |
| A205P (A) | 0.64 | 16245 | 22800 | 0.081 | 0.147 |
| A205P (B) | 0.64 | 13677 | 21644 | 0.062 | 0.138 |
| no lipase (A) | 0.00 | 4200 | 7172 | — | — |
| no lipase (B) | 0.00 | 4247 | 7425 | — | — |

TABLE 36
Malodor Performance Index using CFT C-S-68, chocolate ice cream on cotton, in
Persil Color Gel at 16° C. 300 ppm 3:1 Ca²⁺:Mg²⁺, 30 min, 5 mM HEPES (pH 8.0).

| | lipase | butanoic acid | hexanoic acid | butanoic acid | hexanoic acid |
|---|---|---|---|---|---|
| Lipase (replicate A, B) | (ppm) | (peak area) | (peak area) | PI(malodor) | PI(malodor) |
| Lipex 100L (A) | 0.32 | 31615 | 25972 | 1.000 | 1.000 |
| Lipex 100L (B) | 0.32 | 37379 | 29341 | 1.000 | 1.000 |
| Lipase parent (SEQ ID NO: 1) (A) | 0.32 | 15363 | 8395 | 0.436 | 0.273 |
| Lipase parent (SEQ ID NO: 1 (B) | 0.32 | 15589 | 8830 | 0.377 | 0.271 |
| N146H_A178K_I179K_G268K (A) | 0.32 | 13804 | 6687 | 0.382 | 0.202 |
| N146H_A178K_I179K_G268K (B) | 0.32 | 13860 | 6963 | 0.328 | 0.205 |
| T4K_N146H_I179K_L203R_L212R_N280K (A) | 0.32 | 17511 | 9513 | 0.511 | 0.319 |
| T4K_N146H_I179K_L203R_L212R_N280K (B) | 0.32 | 16884 | 9495 | 0.414 | 0.295 |
| no lipase (A) | 0 | 2782 | 1797 | — | — |
| no lipase (B) | 0 | 2379 | 1205 | — | — |

TABLE 37
Malodor Performance Index using CFT C-S-68, chocolate ice cream on cotton, in
Persil Color Gel at 16° C., 300 ppm 3:1 Ca²⁺:Mg²⁺, 30 min, 5 mM HEPES (pH 8.0).

| | lipase | butanoic acid | hexanoic acid | butanoic acid | hexanoic acid |
|---|---|---|---|---|---|
| Lipase (replicate A, B) | (ppm) | (peak area) | (peak area) | PI(malodor) | PI(malodor) |
| Lipex 100L (A) | 0.64 | 60639 | 44270 | 1.000 | 1.000 |
| Lipex 100L (B) | 0.64 | 65369 | 49305 | 1.000 | 1.000 |
| Lipase parent (SEQ ID NO: 1) (A) | 0.64 | 21157 | 10681 | 0.318 | 0.209 |
| Lipase parent (SEQ ID NO: 1 (B) | 0.64 | 19295 | 10208 | 0.269 | 0.187 |
| N146H_A178K_I179K_G268K (A) | 0.64 | 16592 | 8873 | 0.239 | 0.167 |
| N146H_A178K_I179K_G268K (B) | 0.64 | 24923 | 12300 | 0.358 | 0.231 |
| T4K_N146H_I179K_L203R_L212R_N280K (A) | 0.64 | 23137 | 12189 | 0.352 | 0.245 |
| T4K_N146H_I179K_L203R_L212R_N280K (B) | 0.64 | 25647 | 14544 | 0.369 | 0.277 |
| no lipase (A) | 0 | 2782 | 1797 | — | — |
| no lipase (B) | 0 | 2379 | 1205 | — | — |

### Example 7

### Thermostability of Lipase Variants

The thermostability of lipase variants of the parent lipase of SEQ ID NO:1 described herein was determined as described in Example 2. Lipase variants were subjected to a 30-minute period of heat stress at 45 TC. Activity levels measured for unstressed controls were compared to activity levels after heat stress. The ratio of stressed vs unstressed values was used to calculate the % residual activity and compared to the % residual activity of the parent lipase (SEQ ID NO: 1). The relative increase in thermostability is expressed as a performance index (PI) where PI (thermostability)=% residual activity variant/% residual activity of parent lipase of SEQ ID NO: 1.

TABLE 38
Lipase Thermostability at 45° C., 30 minutes, in HDL

| Lipase variant (positions modified | PI(Thermostability) |
|---|---|
| relative to parent of SEQ ID NO: 1) | 45 C. |
| N146H_I179K | 1.22 |
| N146H_N280K | 1.61 |
| T4K_L212R | 1.75 |
| N146H_A178K_I179K_G268K | 0.10 |
| A178K_I179K_L203R_L212R | 1.90 |
| T4K_N146H_I179K_L203R_L212R_N280K | 1.94 |
| T4K_N146H_A178K_L203R_G268K_N280K | 1.50 |
| K94D_N149D_A178D_K185D_G202E_N280E | 0.22 |
| A29R | 1.51 |
| V70A | 1.44 |
| S95N | 1.10 |
| S227T | 1.81 |
| I272V | 1.71 |
| A159D | 1.29 |
| G206D_G209A | 2.45 |
| G206E_G209A | 2.17 |
| G206E_G209D | 3.00 |
| G206E | 1.82 |
| Parent lipase of SEQ ID NO: 1 (reference) | 1.00 |

### Example 8

### Calcium Ion Binding Stability of Lipase Variants

The calcium ion binding stability of lipase variants of the parent lipase of SEQ ID NO:1 described herein was determined as described in Example 2. Calcium ion binding stability was assessed by incubating samples comprising the lipase variants in the same base HDL formulation as the thermo-stressed samples except the HDL formulation was supplemented with 10 mM EDTA. All samples were incubated for a total of 30 minutes on ice and compared to paired samples incubated on ice without EDTA.

The performance index for calcium ion binding stability, PI(calcium ion binding stability) was calculated as follows:

PI (calcium ion binding stability)=% residual activity variant/% residual activity of parent lipase of SEQ ID NO: 1.

TABLE 39
Calcium ion binding stability of lipase variants, PI(EDTA)

| Lipase variants (positions modified | |
|---|---|
| relative to parent of SEQ ID NO: 1) | PI(EDTA) |
| parent Lipase of SEQ ID NO: 1 | 1.0 |
| N146H_I179K | 1.2 |
| N146H_N280K | 0.5 |
| T4K_N146H_I179K_L203R_L212R_N280K | 4.8 |
| T4K_N146H_A178K_L203R_G268K_N280K | 0.5 |
| A159D | 1.6 |
| G206D_G209A | 4.4 |
| G206E_G209A | 4.3 |
| G206E_G209D | 21.9 |
| N164R_A159D | 1.5 |
| A159S | 2.1 |
| N164D | 1.5 |

### Example 9

Lipolytic Enzyme Variants with Improved Stability in Liquid

Single amino acid variants were made of the parent lipase SEQ ID NO: 2 disclosed herein. To assess the detergent stability of variants of parent lipase SEQ ID NO: 2 in a liquid detergent formulation, detergent stability was determined by the Thermostability Assay as described in Example 2. This Thermostability Assay measures the activity of a lipase after incubation in a liquid detergent with a heat stress versus a condition without heat stress, and therefore represents an accelerated decay assay for measuring the stability of a lipase in a liquid detergent. The relative increase in detergent stability is expressed as a performance index (PI), where PI (detergent stability)=% residual activity variant/% residual activity of parent lipase SEQ ID NO:2. A list of lipase variants showing an increased stability in liquid detergent is shown in Table 40.

TABLE 40
Lipase variants with Improved Detergent
Stability in Liquid Detergent.

| | WT-AA | Position | | Detergent |
|---|---|---|---|---|
| Lipase Variant | (SEQ ID | relative to | | Stability |
| (Mutation) | NO: 2) | SEQ ID NO: 2 | Mutant -AA | (PI) |
| S002V | S | 2 | V | 1.11 |
| H011A | H | 11 | A | 1.59 |
| H011K | H | 11 | K | 1.82 |
| L013C | L | 13 | C | 1.13 |
| L013F | L | 13 | F | 1.14 |
| L013W | L | 13 | W | 1.25 |
| S014C | S | 14 | C | 1.27 |
| S014G | S | 14 | G | 1.19 |
| S014L | S | 14 | L | 1.24 |
| S014W | S | 14 | W | 1.24 |
| D017A | D | 17 | A | 1.14 |
| D017G | D | 17 | G | 1.25 |
| D017S | D | 17 | S | 1.13 |
| D017T | D | 17 | T | 1.22 |
| D018A | D | 18 | A | 1.13 |
| D018F | D | 18 | F | 1.25 |
| D018G | D | 18 | G | 1.39 |
| D018I | D | 18 | I | 1.32 |
| D018K | D | 18 | K | 1.12 |
| D018L | D | 18 | L | 1.37 |
| D018M | D | 18 | M | 1.24 |
| D018N | D | 18 | N | 1.16 |
| D018P | D | 18 | P | 1.36 |
| D018R | D | 18 | R | 1.10 |
| D018T | D | 18 | T | 1.14 |
| D018V | D | 18 | V | 1.16 |
| D018W | D | 18 | W | 1.37 |
| I019C | I | 19 | C | 1.47 |
| V020G | V | 20 | G | 1.16 |
| Y022F | Y | 22 | F | 2.02 |
| Y022I | Y | 22 | I | 1.98 |
| Y022V | Y | 22 | V | 1.12 |
| Y022W | Y | 22 | W | 1.28 |
| G027K | G | 27 | K | 1.22 |
| I028V | I | 28 | V | 1.10 |
| D030A | D | 30 | A | 1.10 |
| D030K | D | 30 | K | 1.22 |
| D030R | D | 30 | R | 1.24 |
| A031G | A | 31 | G | 1.15 |
| E033I | E | 33 | I | 1.11 |
| E033K | E | 33 | K | 1.10 |
| S043K | S | 43 | K | 1.49 |
| S043R | S | 43 | R | 1.24 |
| L044Q | L | 44 | Q | 1.11 |
| A046G | A | 46 | G | 1.32 |
| A046S | A | 46 | S | 1.28 |
| A046T | A | 46 | T | 1.22 |
| F047C | F | 47 | C | 1.37 |
| F047Y | F | 47 | Y | 1.34 |
| S049D | S | 49 | D | 1.13 |
| S049T | S | 49 | T | 1.10 |
| N050P | N | 50 | P | 1.15 |
| V052F | V | 52 | F | 1.32 |
| V052K | V | 52 | K | 1.10 |
| V052M | V | 52 | M | 1.18 |
| R053I | R | 53 | I | 1.23 |
| R053M | R | 53 | M | 1.42 |
| R053Q | R | 53 | Q | 1.38 |
| R053T | R | 53 | T | 1.11 |
| R053V | R | 53 | V | 1.13 |
| L057C | L | 57 | C | 1.16 |
| F060K | F | 60 | K | 1.32 |
| Q062A | Q | 62 | A | 1.13 |
| L065H | L | 65 | H | 1.10 |
| E067I | E | 67 | I | 1.11 |
| E067Q | E | 67 | Q | 1.10 |
| E067R | E | 67 | R | 1.13 |
| E067S | E | 67 | S | 1.11 |
| E067T | E | 67 | T | 1.11 |
| T068S | T | 68 | S | 1.10 |
| A070V | A | 70 | V | 1.12 |
| A070Y | A | 70 | Y | 1.10 |
| L083M | L | 83 | M | 1.19 |
| C085A | C | 85 | A | 1.18 |
| C085G | C | 85 | G | 1.23 |
| K091D | K | 91 | D | 1.14 |
| K091E | K | 91 | E | 1.10 |
| K091N | K | 91 | N | 1.12 |
| K091Q | K | 91 | Q | 1.14 |
| A093E | A | 93 | E | 1.10 |
| S095P | S | 95 | P | 1.13 |
| V099I | V | 99 | I | 1.10 |
| V105A | V | 105 | A | 1.16 |
| V105C | V | 105 | C | 1.14 |
| V105P | V | 105 | P | 1.29 |
| R117A | R | 117 | A | 1.44 |
| R117C | R | 117 | C | 1.38 |
| R117D | R | 117 | D | 1.89 |
| R117E | R | 117 | E | 1.89 |
| R117G | R | 117 | G | 1.38 |
| R117I | R | 117 | I | 1.13 |
| R117K | R | 117 | K | 1.10 |
| R117N | R | 117 | N | 1.34 |
| R117Q | R | 117 | Q | 1.49 |
| R117S | R | 117 | S | 1.42 |
| R117T | R | 117 | T | 1.27 |
| R117V | R | 117 | V | 1.20 |
| I118D | I | 118 | D | 1.38 |
| I118E | I | 118 | E | 1.27 |
| I118G | I | 118 | G | 1.18 |
| I118H | I | 118 | H | 1.14 |
| I118N | I | 118 | N | 1.23 |
| I118S | I | 118 | S | 1.17 |
| M119C | M | 119 | C | 1.36 |
| M119V | M | 119 | V | 1.19 |
| M119Y | M | 119 | Y | 1.16 |
| R120A | R | 120 | A | 1.17 |
| R120C | R | 120 | C | 1.21 |
| R120E | R | 120 | E | 1.50 |
| R120F | R | 120 | F | 1.11 |
| R120G | R | 120 | G | 1.13 |
| R120H | R | 120 | H | 1.25 |
| R120I | R | 120 | I | 1.46 |
| R120K | R | 120 | K | 1.15 |
| R120L | R | 120 | L | 1.41 |
| R120M | R | 120 | M | 1.34 |
| R120Q | R | 120 | Q | 1.29 |
| R120T | R | 120 | T | 1.20 |
| R120V | R | 120 | V | 1.29 |
| R120W | R | 120 | W | 1.14 |
| K121A | K | 121 | A | 1.17 |
| K121E | K | 121 | E | 2.35 |
| K121N | K | 121 | N | 1.42 |
| K121Q | K | 121 | Q | 1.45 |
| K121S | K | 121 | S | 1.30 |
| K121T | K | 121 | T | 1.38 |
| P125D | P | 125 | D | 1.34 |
| P125E | P | 125 | E | 1.34 |
| P125F | P | 125 | F | 1.30 |
| Y127C | Y | 127 | C | 1.10 |
| Y127D | Y | 127 | D | 1.70 |
| Y127E | Y | 127 | E | 1.67 |
| Y127G | Y | 127 | G | 1.13 |
| Y127H | Y | 127 | H | 1.22 |
| Y127N | Y | 127 | N | 1.12 |
| I128A | I | 128 | A | 1.19 |
| I128C | I | 128 | C | 1.21 |
| I128G | I | 128 | G | 1.17 |
| I128K | I | 128 | K | 1.25 |
| I128M | I | 128 | M | 1.10 |
| I128N | I | 128 | N | 1.24 |
| I128Q | I | 128 | Q | 1.45 |
| I128T | I | 128 | T | 1.37 |
| I128Y | I | 128 | Y | 1.15 |
| A129S | A | 129 | S | 1.13 |
| D130C | D | 130 | C | 1.18 |
| A131D | A | 131 | D | 1.81 |
| A131E | A | 131 | E | 1.63 |
| A131N | A | 131 | N | 1.10 |
| A131P | A | 131 | P | 1.40 |
| A131Q | A | 131 | Q | 1.16 |
| A131S | A | 131 | S | 1.14 |
| A131T | A | 131 | T | 1.10 |
| V132T | V | 132 | T | 1.15 |
| K134C | K | 134 | C | 1.32 |
| K134D | K | 134 | D | 1.42 |
| K134E | K | 134 | E | 1.51 |
| K134F | K | 134 | F | 1.24 |
| K134G | K | 134 | G | 1.29 |
| K134I | K | 134 | I | 1.22 |
| K134M | K | 134 | M | 1.25 |
| K134N | K | 134 | N | 1.18 |
| K134Q | K | 134 | Q | 1.21 |
| K134S | K | 134 | S | 1.28 |
| K134T | K | 134 | T | 1.25 |
| K134V | K | 134 | V | 1.25 |
| K134W | K | 134 | W | 1.44 |
| K134Y | K | 134 | Y | 1.15 |
| A135E | A | 135 | E | 1.12 |
| G137R | G | 137 | R | 1.20 |
| T138I | T | 138 | I | 1.10 |
| T138V | T | 138 | V | 1.12 |
| I140E | I | 140 | E | 1.40 |
| I140F | I | 140 | F | 1.26 |
| I140T | I | 140 | T | 1.19 |
| I140V | I | 140 | V | 1.26 |
| S141M | S | 141 | M | 1.21 |
| N146M | N | 146 | M | 1.13 |
| R147P | R | 147 | P | 1.14 |
| P150D | P | 150 | D | 1.18 |
| P150E | P | 150 | E | 1.20 |
| P150T | P | 150 | T | 1.31 |
| I154M | I | 154 | M | 1.17 |
| I154N | I | 154 | N | 1.39 |
| I154Q | I | 154 | Q | 1.20 |
| I154T | I | 154 | T | 1.14 |
| I154V | I | 154 | V | 1.14 |
| A155E | A | 155 | E | 1.14 |
| A155M | A | 155 | M | 1.18 |
| A159D | A | 159 | D | 1.20 |
| L160A | L | 160 | A | 1.50 |
| L160C | L | 160 | C | 1.17 |
| L160S | L | 160 | S | 1.21 |
| L160T | L | 160 | T | 1.12 |
| L160V | L | 160 | V | 1.19 |
| N164A | N | 164 | A | 1.26 |
| N164E | N | 164 | E | 1.35 |
| N164Q | N | 164 | Q | 1.12 |
| N164S | N | 164 | S | 1.23 |
| M166A | M | 166 | A | 1.10 |
| M166D | M | 166 | D | 1.15 |
| M166E | M | 166 | E | 1.24 |
| K170A | K | 170 | A | 1.13 |
| K170D | K | 170 | D | 1.10 |
| K170H | K | 170 | H | 1.10 |
| K170Q | K | 170 | Q | 1.11 |
| K170T | K | 170 | T | 1.10 |
| K171C | K | 171 | C | 1.12 |
| K171D | K | 171 | D | 1.11 |
| K171E | K | 171 | E | 1.15 |
| K171Q | K | 171 | Q | 1.12 |
| G175A | G | 175 | A | 1.13 |
| A178D | A | 178 | D | 1.12 |
| I179E | I | 179 | E | 1.11 |
| R180E | R | 180 | E | 1.14 |
| R180P | R | 180 | P | 1.17 |
| K185C | K | 185 | C | 1.14 |
| K185D | K | 185 | D | 1.16 |
| K185E | K | 185 | E | 1.18 |
| K185L | K | 185 | L | 1.10 |
| K185S | K | 185 | S | 1.11 |
| N189D | N | 189 | D | 1.11 |
| F196I | F | 196 | I | 1.16 |
| Y199F | Y | 199 | F | 1.10 |
| L203C | L | 203 | C | 1.87 |
| L203K | L | 203 | K | 1.90 |
| L203N | L | 203 | N | 1.80 |
| L203P | L | 203 | P | 1.20 |
| L203R | L | 203 | R | 2.35 |
| L203S | L | 203 | S | 2.11 |
| L203T | L | 203 | T | 1.83 |
| I204Y | I | 204 | Y | 1.81 |
| A205C | A | 205 | C | 1.40 |
| A205F | A | 205 | F | 2.57 |
| A205I | A | 205 | I | 2.45 |
| A205L | A | 205 | L | 1.55 |
| A205M | A | 205 | M | 1.40 |
| A205V | A | 205 | V | 2.01 |
| A205W | A | 205 | W | 2.50 |
| A205Y | A | 205 | Y | 1.69 |
| G206C | G | 206 | C | 1.41 |
| G206D | G | 206 | D | 1.13 |
| G206E | G | 206 | E | 1.68 |
| G206Q | G | 206 | Q | 1.11 |
| K208E | K | 208 | E | 1.44 |
| K208G | K | 208 | G | 1.78 |
| K208M | K | 208 | M | 1.60 |
| K208Q | K | 208 | Q | 2.06 |
| G209A | G | 209 | A | 1.68 |
| G209C | G | 209 | C | 1.66 |
| G209D | G | 209 | D | 2.39 |
| G209E | G | 209 | E | 1.13 |
| G209Q | G | 209 | Q | 1.21 |
| L212K | L | 212 | K | 1.48 |
| A217F | A | 217 | F | 2.13 |
| A217I | A | 217 | I | 1.37 |
| A217L | A | 217 | L | 1.26 |
| A217V | A | 217 | V | 1.24 |
| A217Y | A | 217 | Y | 3.31 |
| A218C | A | 218 | C | 1.30 |
| V221F | V | 221 | F | 1.17 |
| V221T | V | 221 | T | 1.16 |
| S223G | S | 223 | G | 1.33 |
| A224G | A | 224 | G | 1.37 |
| A224H | A | 224 | H | 1.89 |
| A224K | A | 224 | K | 1.95 |
| A224Q | A | 224 | Q | 1.45 |
| A224R | A | 224 | R | 1.98 |
| A224S | A | 224 | S | 1.56 |
| A224W | A | 224 | W | 1.48 |
| A224Y | A | 224 | Y | 1.18 |
| S227D | S | 227 | D | 1.53 |
| S227I | S | 227 | I | 1.13 |
| S227N | S | 227 | N | 1.11 |
| S227V | S | 227 | V | 1.17 |
| R229A | R | 229 | A | 1.45 |
| R229C | R | 229 | C | 1.90 |
| R229D | R | 229 | D | 1.48 |
| R229E | R | 229 | E | 1.64 |
| R229G | R | 229 | G | 1.10 |
| R229H | R | 229 | H | 1.29 |
| R229I | R | 229 | I | 1.42 |
| R229K | R | 229 | K | 1.13 |
| R229L | R | 229 | L | 1.35 |
| R229M | R | 229 | M | 1.37 |
| R229N | R | 229 | N | 1.35 |
| R229P | R | 229 | P | 1.49 |
| R229Q | R | 229 | Q | 1.51 |
| R229S | R | 229 | S | 1.40 |
| R229T | R | 229 | T | 1.42 |
| R229V | R | 229 | V | 1.54 |
| K247R | K | 247 | R | 1.11 |
| A251P | A | 251 | P | 1.18 |
| E252Q | E | 252 | Q | 1.82 |
| L255C | L | 255 | C | 1.51 |
| V258I | V | 258 | I | 1.37 |
| V261L | V | 261 | L | 1.53 |
| A262C | A | 262 | C | 1.47 |
| A262F | A | 262 | F | 1.40 |
| A262L | A | 262 | L | 1.27 |
| A262M | A | 262 | M | 1.72 |
| L264K | L | 264 | K | 1.20 |
| G268D | G | 268 | D | 1.62 |
| I272G | I | 272 | G | 1.13 |
| I272K | I | 272 | K | 1.50 |
| I272P | I | 272 | P | 2.08 |
| I272R | I | 272 | R | 1.51 |
| I272V | I | 272 | V | 1.21 |

### Example 10

Lipolytic Enzyme Variants of SEQ ID NO:1 and SEQ ID NO:2 with Improved Stability in Liquid Detergents

As described herein, parent lipases SEQ ID NO: 1 and SEQ ID NO:2 only differ from each other in one amino acid. SEQ ID NO: 1 has a valine at position 70 while SEQ ID NO: 2 has an alanine at position 70. Lipolytic enzyme variants were created from both wild type backbones SEQ ID NO: 1 and SEQ ID NO:2 and the detergent stability of lipase variants relative to the corresponding parent lipase was determined in a liquid detergent formulation by the Thermostability Assay as described in Example 2. Table 41 shows that the same mutations in both backbones resulted in similar increases in Performance Index (detergent stability).

TABLE 41
Lipase variants of SEQ ID NO: 1 and Lipase variants of SEQ ID NO: 2 showing increased stability in HDL.

| | PI (detergent stability) | PI (detergent stability) |
|---|---|---|
| | relative to | relative to |
| Lipase Variant (Mutation) | SEQ ID NO: 2 | SEQ ID NO: 1 |
| Wild-type (SEQ ID NO: 2 or 1 respectively) | 1.00 | 1.00 |
| A159D | 1.20 | 1.25 |
| G206E_G209D | 3.08 | 2.00 |
| T4K_N146H_I179K_L203R_L212R_N280K | 2.21 | 2.50 |
| A159D_G206E_G209D | 3.36 | 2.95 |
| T4K_N146H_A159D_I179K_L203R_L212R_N280K | 2.38 | 2.21 |
| T4K_N146H_I179K_L203R_G206E_G209D_L212R_N280K | 2.56 | 2.74 |
| T4K_N146H_A159D_I179K_L203R_G206E_G209D_L212R_N280K | 2.54 | 2.73 |

### Example 11

### Detergent Stability of Lipase Variants

Crude Bacilus subtilis supernatant containing lipase was ultra-concentrated using 3 kDa molecular weight cut off filtration (Cat. No. UFC9003 EMD Millipore). The concentrated material was formulated in 10 mM HEPES (pH 8.0), 250 ppm 3:1 Ca²⁺:Mg²⁺ hardness, and 16% glycerol. A 1% (v/v) of the formulated material was added to full strength HDL Persil Color Gel detergent and incubated at 30° C. The lipase activity was assessed in timepoints over a period of 10 days using 4-methylumbelliferone caprylate as a substrate. The lipase samples were each diluted with an emulsion to a final concentration of 1 mM 4-methylumbelliferone caprylate, 0.2% Triton X-100, 10 mM HEPES (pH 8.0), 250 ppm 3:1 Ca²⁺:Mg²⁺ hardness. Samples were added to a Corning® 96 Well Solid Polystyrene Microplate (Sigma Cat. No. CLS3915). The release of 4-methylumbelliferone due to hydrolysis was monitored by measuring the fluorescence with the excitation at 380 nm and the emission at 450 nm for 5 minutes in 20 second intervals with a SpectraMax (Molecular Devices). Percent Lipase activity remaining in HDL Stability study after a certain time point (1 day, 3 days, and 10 days) are shown in Table 42,

TABLE 42
Percent lipase activity remaining in HDL Stability study
for lipase variants of parent lipase of SEQ ID NO: 2.

| | | | | |
|---|---|---|---|---|
| | Parent | | | |
| Time | (SEQ ID NO: 2) | Y22F | L203R | A217Y |

| | | | | | |
|---|---|---|---|---|---|
| 1 | day | 14.7 | 51.7 | 77.3 | 61.1 |
| 3 | days | 2.9 | 27.7 | 32.4 | 82.7 |
| 10 | days | 0.8 | 2.4 | 3.6 | 44.3 |

Table 42 shows that the Lipase variants Y22F, L203R and A217Y had an increased stability when compared to the reference lipase of SEQ ID NO:2.

That which is claimed:

1. A lipolytic enzyme variant or an active fragment thereof comprising an amino acid modification to a parent lipolytic enzyme, wherein the amino acid modification is at a position of the lipolytic enzyme variant selected from the group consisting of 2, 4, 11, 13, 14, 17, 18, 19, 20, 22, 27, 28, 29, 30, 31, 33, 43, 44, 46, 47, 49, 50, 52, 53, 57, 60, 62, 64, 65, 67, 68, 70, 83, 85, 91, 93, 95, 99, 105, 117, 118, 119, 120, 121, 122, 125, 127, 128, 130, 131, 132, 134, 135, 137, 138, 140, 141, 146, 147, 150, 154, 155, 159, 160, 164, 166, 170, 171, 175, 178, 179, 180, 185, 189, 196, 199, 203, 204, 205, 206, 208, 209, 212, 217, 218, 221, 223, 224, 227, 229, 247, 251, 252, 255, 258, 261, 262, 264, 267, 268, 272 and 280, wherein the amino acid positions of the variant are numbered by correspondence with the amino acid sequence of SEQ ID NO: 2, wherein the lipolytic enzyme variant has at least 60% sequence identity to SEQ ID NO: 2.

2. The lipolytic enzyme variant of claim 1, wherein said variant is derived from a parent lipolytic enzyme having 60%, 65%, 70%, 75%, 80%, 85%, 86%, 87%, 88%, 89%, 90%, 91%, 92%, 93%, 94%, 95%, %, 97%, 98%, 99%, or 100% amino acid sequence identity to the amino acid sequence of SEQ ID NO: 2.

3. The lipolytic enzyme variant of claim 1, wherein said variant comprises an amino acid sequence having 60%, 65%, 70%, 75%, 80%, 85%, 86%, 87%, 88%, 89%, 90%, 91%, 92%, 93%, 94%, 95%, 96%, 97%, 98% or 99% amino acid sequence identity to the amino acid sequence of SEQ ID NO: 2.

4. The lipolytic enzyme variant of claim 1, wherein the amino acid modification is a substitution selected from the group consisting of S002V, T4K, H011A, H011K, L013C, L013F, L013W, S014C, S014G, S014L, S014W, D017A, D017G, D017S, D017T, D018A, D018F, D018G, D018I, D018K, D018L, D018M, D018N, D018P, D018R, D018T, D018V, D018W, I019C, V020G, Y022F, Y022I, Y022V, Y022W, G027K, I028V, A29R, D030A, D030K, D030R, A031G, E033I, E033K, S043K, S043R, L044Q, A046G, A046S, A046T, F047C, F047Y, S049D, S049T, N050P, V052F, V052K, V052M, R053I, R053M, R053Q, R053T, R053V, L057C, F060K, Q062A, I64V, L065H, E067I, E067Q, E067R, E067S, E067T, T068S, A070V, A070Y, L083M, C085A, C085G, K091D, K091E, K091N, K091Q, A093E, S095P, S095N, V099I, V105A, V105C, V105P, R117A, R117C, R117D, R117E, R117G, R117I, R117K, R117N, R117Q, R117S, R117T, R117V, I118D, I118E, I118G, I118H, I118N, I118S, M119C, M119V, M119Y, R120A, R120C, R120E, R120F, R120G, R120H, R120I, R120K, R120L, R120M, R120Q, R120T, R120V, R120W, K121A, K121E, K121N, K121Q, K121S, K121T, D122P, P125D, P125E, P125F, Y127C, Y127D, Y127E, Y127G, Y127H, Y127N, I128A, I128C, I128G, I128K, I128M, I128N, I128Q, I128T, I128Y, A129S, D130C, A131D, A131E, A131N, A131P, A131Q, A131S, A131T, V132T, K134C, K134D, K134E, K134F, K134G, K134I, K134M, K134N, K134Q, K134S, K134T, K134V, K134W, K134Y, A135E, G137R, T138I, T138V, I140E, I140F, I140T, I140V, S141M, N146M, N146H, R147P, P150D, P150E, P150T, I154M, I154N, I154Q, I154T, I154V, A155E, A155M, A159D, A159S, L160A, L160C, L160S, L160T, L160V, N164A, N164E, N164Q, N164S, N164R, N164D, M166A, M166D, M166E, K170A, K170D, K170H, K170Q, K170T, K171C, K171D, K171E, K171Q, G175A, A178D, A178K, I179E, I179K, R180E, R180P, K185C, K185D, K185E, K185L, K185S, N189D, F196I, Y199F, L203C, L203K, L203N, L203P, L203R, L203S, L203T, I204Y, A205C, A205F, A205I, A205L, A205M, A205P, A205V, A205W, A205Y, G206C, G206D, G206E, G206Q, K208E, K208G, K208M, K208Q, G209A, G209C, G209D, G209E, G209Q, L212K, L212R, A217F, A217I, A217L, A217V, A217Y, A218C, V221F, V221T, S223G, A224G, A224H, A224K, A224Q, A224R, A224S, A224W, A224Y, S227D, S227I, S227N, S227T, S227V, R229A, R229C, R229D, R229E, R229G, R229H, R229I, R229K, R229L, R229M, R229N, R229P, R229Q, R229S, R229T, R229V, K247R, A251P, E252Q, L255C, V258I, V261L, A262C, A262F, A262L, A262M, L264K, R267P, G268D, G268K, I272G, I272K, I272P, I272R, I272V and N280K.

5. The lipolytic enzyme variant or active fragment thereof of claim 1, wherein said lipolytic enzyme variant or active fragment thereof has an improved performance relative to the parent lipolytic enzyme, wherein the improved performance is selected from the group consisting of an improved wash performance, a decreased malodor, an increased detergent stability, an increased thermostability, an increased calcium ion binding stability, an increased protease stability, and any one combination thereof.

6. The lipolytic enzyme variant or active fragment thereof of claim 5, wherein the improved wash performance is an improved wash performance at a low temperature.

7. The lipolytic enzyme variant or active fragment thereof of claim 5, wherein the variant or active fragment has a wash performance index (PI(wash)) relative to the parent lipolytic enzyme that is greater than 1.0.

8. The lipolytic enzyme variant or active fragment thereof of claim 5, wherein the variant or active fragment has a detergent stability index (PI(detergent stability)) relative to the parent lipolytic enzyme that is greater than 1.0.

9. The lipolytic enzyme variant or active fragment thereof of claim 5, wherein the variant or active fragment has a thermostability performance index (PI(thermostability)) relative to the parent lipolytic enzyme that is greater than 1.0.

10. The lipolytic enzyme variant or active fragment thereof of claim 5, wherein the variant or active fragment has a calcium ion binding stability index (PI(calcium ion binding stability)) relative to the parent lipolytic enzyme that is greater than 1.0.

11. The lipolytic enzyme variant or active fragment thereof of claim 5, wherein the variant or active fragment has a protease stability that is greater that the protease stability of the parent lipolytic enzyme.

12. The lipolytic enzyme variant or active fragment thereof of claim 1, wherein the variant or active fragment has a decreased malodor performance index (PI(malodor) relative to the lipolytic enzyme of SEQ ID NO: 7 that is less than 1.0.

13. The lipolytic enzyme variant or active fragment thereof of claim 1, wherein the variant or active fragment has lipolytic activity.

14. A composition comprising at least one lipolytic enzyme variant or fragment according to claim 1.

15. The composition according to claim 14, wherein said composition is a detergent composition.

16. The composition according to claim 15, wherein said detergent composition is selected from a laundry detergent, a fabric softening detergent, a dishwashing detergent, and a hard-surface cleaning detergent.

17. The composition of claim 14, wherein said composition further comprises one or more calcium ions and/or zinc ions; one or more enzyme stabilizers; from about 0.001% to about 1.0 weight % of said lipolytic enzyme variant(s); one or more bleaching agents; one or more adjunct materials; and/or one or more additional enzymes or enzyme derivatives selected from the group consisting of acyl transferases, alpha-amylases, beta-amylases, alpha-galactosidases, arabinosidases, aryl esterases, beta-galactosidases, carrageenases, catalases, cellobiohydrolases, cellulases, chondroitinases, cutinases, DNase or nuclease, endo-beta-1,4-glucanases, endo-beta-mannanases, esterases, exo-mannanases, galactanases, glucoamylases, hemicellulases, hyaluronidases, keratinases, laccases, lactases, ligninases, lipases, lipoxygenases, lysozymes, mannanases, oxidases, pectate lyases, pectin acetyl esterases, pectinases, pentosanases, perhydrolases, peroxidases, phenoloxidases, phosphatases, phospholipases, phytases, polygalacturonases, proteases, pullulanases, reductases, rhamnogalacturonases, beta-glucanases, tannases, transglutaminases, xylan acetyl-esterases, xylanases, xyloglucanases, xylosidases, metalloproteases, nucleases, additional serine proteases, and combinations thereof.

18. The composition of claim 14, wherein said composition is a granular, powder, solid, bar, liquid, tablet, gel, paste or unit dose composition.

19. A DNA sequence encoding the lipolytic enzyme variant or active fragment of claim 1.

20. An expression vector or cassette comprising the DNA sequence of claim 19.

21. The expression vector or cassette of claim 20, wherein the DNA sequence is operably linked to a promoter.

22. A recombinant host cell comprising the DNA sequence of claim 19.

23. Use of the lipolytic enzyme variant or fragment of claim 1 for hydrolyzing a lipolytic enzyme substrate.

24. A method for cleaning a surface comprising contacting the surface with a composition comprising at least one lipolytic enzyme variant of claim 1, and optionally a surfactant.

25. A method of producing a lipolytic enzyme variant comprising culturing the host cell comprising a DNA sequence encoding the lipolytic enzyme variant or active fragment of claim 1 in a culture medium under conditions conducive for the production of the lipolytic enzyme variant and recovering the lipolytic enzyme variant from said culture medium.


SEQUENCE LISTING (7 sequences)

SEQ ID NO: 1 - protein, 287 aa, Artificial Sequence
  synthesized sequence
MSTTYPIVLVHGLSGFDDIVGYPYFYGIADALEKDGHKVFTASLSAFNSNEVRGEQLWEFVQKILKETKVKKVNLIGHSQGPLACRYVAAKHAKSIASVTSVNGVNHGSEIADLVRRIMRKDSVPEYIADAVMKAIGTIISTFSGNRGNPQDAIAALEALTTENVMEFNKKYPQGLPAIRGGEGKEVVNGVHYYSFGSYIQGLIAGEKGNLLDPTHAAMRVLSAFFSERENDGLVGRTSMRLGKLIKDDYAEDHLDMVNQVAGLVGRGEDIIAIYTNHANFLASKKL

SEQ ID NO: 2 - protein, 287 aa, Proteus vulgaris
MSTTYPIVLVHGLSGFDDIVGYPYFYGIADALEKDGHKVFTASLSAFNSNEVRGEQLWEFVQKILKETKAKKVNLIGHSQGPLACRYVAAKHAKSIASVTSVNGVNHGSEIADLVRRIMRKDSVPEYIADAVMKAIGTIISTFSGNRGNPQDAIAALEALTTENVMEFNKKYPQGLPAIRGGEGKEVVNGVHYYSFGSYIQGLIAGEKGNLLDPTHAAMRVLSAFFSERENDGLVGRTSMRLGKLIKDDYAEDHLDMVNQVAGLVGRGEDIIAIYTNHANFLASKKL

SEQ ID NO: 3 - DNA, 861 bp, Artificial sequence
  synthesized sequence
gtgtcaactacatatccaattgttttagttcatggtttatctggttttgatgatatcgtcggttatccttatttttatggtatcgcagacgcattagaaaaagatggtcataaagttttcaccgcatctctttctgcatttaactctaacgaagttcgtggagaacaactttgggaatttgtgcaaaaaattctcaaagaaactaaagttaagaaagtaaatttaatcggtcatagccaaggtccattagcttgtcgttatgttgctgcaaaacatgctaaatctatcgcttctgttacctctgttaatggtgtaaaccacggatcagagattgcggatctagtgagacgtattatgcgcaaagatagcgtacctgaatatattgctgatgcagttatgaaagctattggtactattatttctacattttcaggtaatcgtggtaatccacaagatgctattgctgcattagaagcattaacaactgaaaatgtaatggaatttaacaaaaagtatccacaagggctgccagccattcgtggtggtgaaggtaaagaagtcgtaaatggtgttcattactattcatttggtagttatatccaaggtctgattgccggcgaaaaaggtaatttattggaccctactcatgctgcaatgcgtgtattaagtgcattcttttctgaacgtgaaaatgacggtttggtgggtcgtacaagtatgagattaggtaaactaattaaagatgattatgctgaagatcatctggacatggtaaaccaggttgcaggtctagtagggcgtggtgaagatattattgctatttataccaaccatgcgaatttcttagcaagtaaaaagctg

SEQ ID NO: 4 - DNA, 861 bp, Artificial sequence
  synthesized sequence
gtgtcaacaacatatcctattgtcctggtacacggcctttctggtttcgatgacatcgtaggatacccttatttttatgggattgccgacgccctggagaaagatggccacaaagtttttacagcctcactctctgcattcaattccaacgaagtccgtggcgagcaattatgggagttcgtgcaaaagattctcaaagagactaaagtcaaaaaggtgaatttgatcgggcactcccaaggtcctcttgcgtgtcgttatgtggcggccaagcatgctaaaagtattgcaagtgttacatctgtgaatggagtgaatcacggtagcgaaatcgccgatcttgtcagacggattatgagaaaagattctgtccctgagtatatcgcggacgcggtaatgaaggctattggcactataatcagtacttttagcggaaatagaggaaaccctcaagacgctatagcagctctggaggccttaacgacggaaaacgtgatggaatttaacaaaaaatatcctcagggactgccagcaattcgtgggggtgaaggtaaagaagtcgtgaacggcgtacactactatagctttggttcttacatacagggtctcatcgctggcgagaagggaaacttgctcgatcctacccacgccgctatgcgcgttttatccgcgtttttttcagaacgtgagaacgatggtttagtaggacggacttcaatgcggctcggcaagttaattaaagacgactacgctgaggatcatttagatatggtcaatcaagttgcggggttagttggacgcggggaggatataattgctatatatacgaatcatgccaattttttagcgtcaaaaaagctc

SEQ ID NO: 5 - DNA, 1398 bp, artificial sequence
  Synthesized sequence
gtcgctgataaacagctgacatcaatatcctattttttcaaaaaatattttaaaagttgttgacttaaaagaagctaaatgttatagtaataaaacagaatagtcttttaagtaagtctactctgaatttttttaaaaggagagggtaaagaaagccgccaggaaaaacttgtctgaatagtacggttgcaatttttaggggaaacagatatacttaagtgtacagaatagtcttttaagtaagtctactctgaatttttttaaaaggagagggtaaagagtgtcaacaacatatcctattgtcctggtacacggcctttctggtttcgatgacatcgtaggatacccttatttttatgggattgccgacgccctggagaaagatggccacaaagtttttacagcctcactctctgcattcaattccaacgaagtccgtggcgagcaattatgggagttcgtgcaaaagattctcaaagagactaaagtcaaaaaggtgaatttgatcgggcactcccaaggtcctcttgcgtgtcgttatgtggcggccaagcatgctaaaagtattgcaagtgttacatctgtgaatggagtgaatcacggtagcgaaatcgccgatcttgtcagacggattatgagaaaagattctgtccctgagtatatcgcggacgcggtaatgaaggctattggcactataatcagtacttttagcggaaatagaggaaaccctcaagacgctatagcagctctggaggccttaacgacggaaaacgtgatggaatttaacaaaaaatatcctcagggactgccagcaattcgtgggggtgaaggtaaagaagtcgtgaacggcgtacactactatagctttggttcttacatacagggtctcatcgctggcgagaagggaaacttgctcgatcctacccacgccgctatgcgcgttttatccgcgtttttttcagaacgtgagaacgatggtttagtaggacggacttcaatgcggctcggcaagttaattaaagacgactacgctgaggatcatttagatatggtcaatcaagttgcggggttagttggacgcggggaggatataattgctatatatacgaatcatgccaattttttagcgtcaaaaaagctctaatctagatacataaaaaaccggccttggccccgccggttttttattatttttcttcctccgcatgttcaatccgctccataatcgacggatggctccctctgaaaattttaacgagaaacggcgggttgacccggctcagtcccgtaacggccaagtcctgaaacgtctcaatcgccgcttcccggtttccggtcagctcaatgccgtaacggtcggcggcgttttcctgataccgggagacggcattcgtaatc

SEQ ID NO: 6 - DNA, 1725 bp, artificial sequence
  synthesized sequence
attcctccattttcttctgctatcaaaataacagactcgtgattttccaaacgagctttcaaaaaagcctctgccccttgcaaatcggatgcctgtctataaaattcccgatattggttaaacagcggcgcaatggcggccgcatctgatgtctttgcttggcgaatgttcatcttatttcttcctccctctcaataattttttcattctatcccttttctgtaaagtttatttttcagaatacttttatcatcatgctttgaaaaaatatcacgataatatccattgttctcacggaagcacacgcaggtcatttgaacgaattttttcgacaggaatttgccgggactcaggagcatttaacctaaaaaagcatgacatttcagcataatgaacatttactcatgtctattttcgttcttttctgtatgaaaatagttatttcgagtctctacggaaatagcgagagatgatatacctaaatagagataaaatcatctcaaaaaaatgggtctactaaaatattattccatctattacaataaattcacagaatagtcttttaagtaagtctactctgaatttttttaaaaggagagggtaaagagtgtcaactacatatccaattgttttagttcatggtttatctggttttgatgatatcgtcggttatccttatttttatggtatcgcagacgcattagaaaaagatggtcataaagttttcaccgcatctctttctgcatttaactctaacgaagttcgtggagaacaactttgggaatttgtgcaaaaaattctcaaagaaactaaagttaagaaagtaaatttaatcggtcatagccaaggtccattagcttgtcgttatgttgctgcaaaacatgctaaatctatcgcttctgttacctctgttaatggtgtaaaccacggatcagagattgcggatctagtgagacgtattatgcgcaaagatagcgtacctgaatatattgctgatgcagttatgaaagctattggtactattatttctacattttcaggtaatcgtggtaatccacaagatgctattgctgcattagaagcattaacaactgaaaatgtaatggaatttaacaaaaagtatccacaagggctgccagccattcgtggtggtgaaggtaaagaagtcgtaaatggtgttcattactattcatttggtagttatatccaaggtctgattgccggcgaaaaaggtaatttattggaccctactcatgctgcaatgcgtgtattaagtgcattcttttctgaacgtgaaaatgacggtttggtgggtcgtacaagtatgagattaggtaaactaattaaagatgattatgctgaagatcatctggacatggtaaaccaggttgcaggtctagtagggcgtggtgaagatattattgctatttataccaaccatgcgaatttcttagcaagtaaaaagctgtaatctagatacataaaaaaccggccttggccccgccggttttttattatttttcttcctccgcatgttcaatccgctccataatcgacggatggctccctctgaaaattttaacgagaaacggcgggttgacccggctcagtcccgtaacggccaagtcctgaaacgtctcaatcgccgcttcccggtttccggtcagctcaatgccgtaacggtcggcggcgttttcctgataccgggagacggcattcgtaatc

SEQ ID NO: 7 - protein, 269 aa, Thermomyces lanuginosus [sequence omitted]